(19) United States
(12) Patent Application Publication
(10) Pub. No.: US 2025/0009853 A1
(43) Pub. Date: Jan. 9, 2025

(54) MUTANT ACE2 PROTEINS AND METHODS OF USING SAME

(71) Applicant: Wisconsin Alumni Research Foundation, Madison, WI (US)

(72) Inventors: Philip Romero, Madison, WI (US); Peter Heinzelman, Madison, WI (US)

(73) Assignee: Wisconsin Alumni Research Foundation, Madison, WI (US)

(21) Appl. No.: 18/708,496

(22) PCT Filed: Dec. 2, 2022
(86) PCT No.: PCT/US2022/051606

§ 371 Date: May 8, 2024

Related U.S. Application Data
(60) Provisional application No. 63/285,592, filed on Dec. 3, 2021.

(51) Int. Cl. A61K 38/48; C12N 9/48

(52) U.S. Cl. CPC A61K 38/4813; C12N 9/485; C12Y 304/17023

(57) ABSTRACT

ACE2 mutants and methods of using same. The mutants have one or more mutations with respect to an ACE2 protein represented by positions 18-615 of SEQ ID NO:1, such as a mutation at one or more of positions 145, 149, 273, 347, 360, 363, 371, 504, 510, and 514 of SEQ ID NO:1. The mutants can have enhanced activity and/or specificity in hydrolyzing angiotensin II, among other activities.

### CROSS-REFERENCE TO RELATED APPLICATIONS

Priority is hereby claimed to U.S. Appl. No. 63/285,592, filed Dec. 3, 2021, which is incorporated herein by reference.

### STATEMENT REGARDING FEDERALLY SPONSORED RESEARCH

This invention was made with government support under GM119854 awarded by the National Institutes of Health. The government has certain rights in the invention.

### SEQUENCE LISTING

The instant application contains a Sequence Listing which has been electronically submitted in XML format and is hereby incorporated by reference in its entirety. The XML copy of the Sequence Listing was created on Mar. 14, 2024, is named USPTO-240508-09824505-P210188US02-APP-SEQ_LIST and is 21,860 bytes in size.

### FIELD OF THE INVENTION

The invention is directed to ACE2 mutants, particularly ACE2 mutants having enhanced activity and/or specificity in hydrolyzing angiotensin II, and methods of using same.

### BACKGROUND

ACE2 (angiotensin converting enzyme 2, also known as angiotensin-converting enzyme-related carboxypeptidase; Enzyme Commission Number: 3.4.17.23) is a key metalloprotease of the renin angiotensin system (RAS). ACE2 is a carboxypeptidase that primarily exists as a membrane-anchored zinc metalloprotease, which acts in the blood circulation and induces local effects in affected organs, as well as systemic effects via the blood circulation. ACE2 is expressed in the vascular system as well as in most organs, but predominantly in the kidneys, liver, heart, lungs and testes. It cleaves as a monopeptidase, thereby activating or inactivating a plethora of substrates, including peptides of the RAS such as Angiotensin II (Ang-II), which is cleaved to angiotensin 1-7 (Ang1-7), and Angiotensin I (Ang-I), which is cleaved to Angiotensin 1-9 (Ang1-9), as well as Apelin, Pro-Dynorphin, Des-Arg-Bradykinin, and others (Vickers, C. et al., J. Biol. Chem., 277, 2002, 14838-14843). ACE2 plays a central role within the RAS as a counter-regulator to the ACE/Ang-II/AT1 receptor axis. ACE2 acts by shifting from the pro-inflammatory, hypertensive, proliferative and vasoconstrictive axis, which is mediated by Ang-II, to the counter-regulatory axis triggered by the cleavage of Ang-II by ACE2 to generate Ang1-7, and to a lesser extent, the cleavage of Ang-I by ACE2 to generate Ang1-9. ACE2 is considered to be an important modulator of homeostasis. The catalytic activity of ACE2 is approximately 400-fold higher with Ang-II than with Ang-I. A soluble form of ACE2 is also found in the circulation, which lacks the transmembrane domain. It is this and other soluble forms that are better suited to being used as an active compound in a wide array of therapeutic approaches than the membrane-bound form.

Soluble and recombinant forms of ACE2 have been developed to treat life-threatening diseases such as acute pulmonary and cardiac conditions, as well as fibrotic and oncologic diseases.

Modified forms of ACE2 with enhanced or altered activity are needed for the improved treatment of diseases.

### SUMMARY OF THE INVENTION

One aspect of the invention is directed to an unnatural, mutant ACE2 protein. The protein preferably comprises an amino acid sequence at least 90% identical to positions 18-615 of SEQ ID NO:1. The amino acid sequence preferably comprises one or more of a residue other than glutamate at a position corresponding to position 145 of SEQ ID NO: 1; a residue other than asparagine at a position corresponding to position 149 of SEQ ID NO: 1; a residue other than arginine at a position corresponding to position 273 of SEQ ID NO:1; a residue other than threonine at a position corresponding to position 347 of SEQ ID NO:1; a residue other than methionine at a position corresponding to position 360 of SEQ ID NO: 1; a residue other than lysine at a position corresponding to position 363 of SEQ ID NO:1; a residue other than threonine at a position corresponding to position 371 of SEQ ID NO:1; a residue other than phenylalanine at a position corresponding to position 504 of SEQ ID NO:1; a residue other than tyrosine at a position corresponding to position 510 of SEQ ID NO:1; and a residue other than arginine at a position corresponding to position 514 of SEQ ID NO:1. The protein preferably exhibits activity in hydrolyzing Angiotensin II.

In some versions, the amino acid sequence comprises one or more of: a residue other than glutamate at a position corresponding to position 145 of SEQ ID NO:1; a residue other than asparagine at a position corresponding to position 149 of SEQ ID NO: 1; a residue other than methionine at a position corresponding to position 360 of SEQ ID NO: 1; a residue other than lysine at a position corresponding to position 363 of SEQ ID NO:1; a residue other than threonine at a position corresponding to position 371 of SEQ ID NO: 1; and a residue other than tyrosine at a position corresponding to position 510 of SEQ ID NO:1.

In some versions, the amino acid sequence comprises one or more of: a residue other than methionine at a position corresponding to position 360 of SEQ ID NO:1; a residue other than threonine at a position corresponding to position 371 of SEQ ID NO:1; and a residue other than tyrosine at a position corresponding to position 510 of SEQ ID NO:1. In some versions, the amino acid sequence comprises a residue other than methionine at a position corresponding to position 360 of SEQ ID NO:1. In some versions, the amino acid sequence comprises a residue other than threonine at a position corresponding to position 371 of SEQ ID NO:1. In some versions, the amino acid sequence comprises a residue other than tyrosine at a position corresponding to position 510 of SEQ ID NO:1.

In some versions, the amino acid sequence comprises one or more of leucine, proline, or a conservative variant of leucine or proline at a position corresponding to position 360 of SEQ ID NO: 1; alanine, aspartate, leucine, phenylalanine, serine, or a conservative variant or alanine, aspartate, leucine, phenylalanine, or serine, at a position corresponding to position 371 of SEQ ID NO:1; and isoleucine, valine, or a conservative variant of isoleucine or valine at a position corresponding to position 510 of SEQ ID NO:1. In some versions, the amino acid sequence comprises leucine, proline, or a conservative variant of leucine or proline at a position corresponding to position 360 of SEQ ID NO:1. In some versions, the amino acid sequence comprises alanine, aspartate, leucine, phenylalanine, serine, or a conservative variant or alanine, aspartate, leucine, phenylalanine, or serine, at a position corresponding to position 371 of SEQ ID NO: 1. In some versions, the amino acid sequence comprises isoleucine, valine, or a conservative variant of isoleucine or valine at a position corresponding to position 510 of SEQ ID NO:1.

In some versions, the amino acid sequence comprises one or more of: leucine or proline at a position corresponding to position 360 of SEQ ID NO:1; alanine, aspartate, leucine, phenylalanine, or serine at a position corresponding to position 371 of SEQ ID NO:1; and isoleucine or valine at a position corresponding to position 510 of SEQ ID NO:1. In some versions, the amino acid sequence comprises leucine or proline at a position corresponding to position 360 of SEQ ID NO:1. In some versions, the amino acid sequence comprises alanine, aspartate, leucine, phenylalanine, or serine at a position corresponding to position 371 of SEQ ID NO:1. In some versions, the amino acid sequence comprises isoleucine or valine at a position corresponding to position 510 of SEQ ID NO:1.

In some versions, the amino acid sequence comprises one or more of: leucine at a position corresponding to position 360 of SEQ ID NO:1; leucine at a position corresponding to position 371 of SEQ ID NO:1; and isoleucine at a position corresponding to position 510 of SEQ ID NO:1. In some versions, the amino acid sequence comprises leucine at a position corresponding to position 360 of SEQ ID NO:1.

In some versions, the amino acid sequence comprises leucine at a position corresponding to position 371 of SEQ ID NO:1. In some versions, the amino acid sequence comprises isoleucine at a position corresponding to position 510 of SEQ ID NO:1.

In some versions, the amino acid sequence comprises two or more of a residue other than methionine at a position corresponding to position 360 of SEQ ID NO:1; a residue other than threonine at a position corresponding to position 371 of SEQ ID NO:1; and a residue other than tyrosine at a position corresponding to position 510 of SEQ ID NO:1.

In some versions, the amino acid sequence comprises two or more of: leucine, proline, or a conservative variant of leucine or proline at a position corresponding to position 360 of SEQ ID NO:1; alanine, aspartate, leucine, phenylalanine, serine, or a conservative variant or alanine, aspartate, leucine, phenylalanine, or serine, at a position corresponding to position 371 of SEQ ID NO:1; and isoleucine, valine, or a conservative variant of isoleucine or valine at a position corresponding to position 510 of SEQ ID NO:1.

In some versions, the amino acid sequence comprises two or more of: leucine or proline at a position corresponding to position 360 of SEQ ID NO:1; alanine, aspartate, leucine, phenylalanine, or serine at a position corresponding to position 371 of SEQ ID NO:1; and isoleucine or valine at a position corresponding to position 510 of SEQ ID NO:1.

In some versions, the amino acid sequence comprises two or more of leucine at a position corresponding to position 360 of SEQ ID NO:1; leucine at a position corresponding to position 371 of SEQ ID NO:1; and isoleucine at a position corresponding to position 510 of SEQ ID NO:1.

In some versions, the amino acid sequence comprises a residue other than tyrosine at a position corresponding to position 510 of SEQ ID NO:1. In some versions, the amino acid sequence further comprises one or more of: a residue other than methionine at a position corresponding to position 360 of SEQ ID NO:1; and a residue other than threonine at a position corresponding to position 371 of SEQ ID NO:1. In some versions, the amino acid sequence further comprises a residue other than methionine at a position corresponding to position 360 of SEQ ID NO:1. In some versions, the amino acid sequence further comprises a residue other than threonine at a position corresponding to position 371 of SEQ ID NO:1.

In some versions, the amino acid sequence comprises isoleucine, valine, or a conservative variant of isoleucine or valine at a position corresponding to position 510 of SEQ ID NO:1. In some versions, the amino acid sequence further comprises one or more of leucine, proline, or a conservative variant of leucine or proline at a position corresponding to position 360 of SEQ ID NO:1; and alanine, aspartate, leucine, phenylalanine, serine, or a conservative variant or alanine, aspartate, leucine, phenylalanine, or serine, at a position corresponding to position 371 of SEQ ID NO: 1. In some versions, the amino acid sequence further comprises leucine, proline, or a conservative variant of leucine or proline at a position corresponding to position 360 of SEQ ID NO:1. In some versions, the amino acid sequence further comprises alanine, aspartate, leucine, phenylalanine, serine, or a conservative variant or alanine, aspartate, leucine, phenylalanine, or serine, at a position corresponding to position 371 of SEQ ID NO:1.

In some versions, the amino acid sequence comprises isoleucine or valine at a position corresponding to position 510 of SEQ ID NO:1. In some versions, the amino acid sequence further comprises one or more of leucine or proline at a position corresponding to position 360 of SEQ ID NO:1; and alanine, aspartate, leucine, phenylalanine, or serine at a position corresponding to position 371 of SEQ ID NO:1. In some versions, the amino acid sequence further comprises leucine or proline at a position corresponding to position 360 of SEQ ID NO:1. In some versions, the amino acid sequence further comprises alanine, aspartate, leucine, phenylalanine, or serine at a position corresponding to position 371 of SEQ ID NO:1.

In some versions, the amino acid sequence comprises isoleucine at a position corresponding to position 510 of SEQ ID NO:1. In some versions, the amino acid sequence further comprises one or more of leucine at a position corresponding to position 360 of SEQ ID NO:1; and leucine at a position corresponding to position 371 of SEQ ID NO:1. In some versions, the amino acid sequence further comprises leucine at a position corresponding to position 360 of SEQ ID NO:1. In some versions, the amino acid sequence further comprises leucine at a position corresponding to position 371 of SEQ ID NO:1.

In some versions, the amino acid sequence is at least 95% identical to positions 18-615 of SEQ ID NO:1. In some versions, the amino acid sequence is at least 99% identical to positions 18-615 of SEQ ID NO:1.

In some versions, the protein exhibits at least one of: an increase in k_{cat} in hydrolyzing Angiotensin II; a decrease in Kₘ in hydrolyzing Angiotensin II; an increase in k_{cat}/Kₘ in hydrolyzing Angiotensin II; an increase in Angiotensin II:Apelin-13 hydrolysis ratio; a decrease in k_{cat}/Kₘ in hydrolyzing Apelin-13; and a decrease in k_{cat}/Kₘ in hydrolyzing Des-Arg9-bradykinin, with respect to a protein comprising an amino acid sequence 100% identical to positions 18-615 of SEQ ID NO:1. In some versions, the protein exhibits an increase in k_{cat} in hydrolyzing Angiotensin II with respect to a protein comprising an amino acid sequence 100% identical to positions 18-615 of SEQ ID NO:1. In some versions, the protein exhibits a decrease in Kₘ in hydrolyzing Angiotensin II with respect to a protein comprising an amino acid sequence 100% identical to positions 18-615 of SEQ ID NO:1. In some versions, the protein exhibits an increase in k_{cat}/Kₘ in hydrolyzing Angiotensin II with respect to a protein comprising an amino acid sequence 100% identical to positions 18-615 of SEQ ID NO:1. In some versions, the protein exhibits an increase in Angiotensin II:Apelin-13 hydrolysis ratio with respect to a protein comprising an amino acid sequence 100% identical to positions 18-615 of SEQ ID NO:1. In some versions, the protein exhibits a decrease in k_{cat}/Kₘ in hydrolyzing Apelin-13 with respect to a protein comprising an amino acid sequence 100% identical to positions 18-615 of SEQ ID NO:1. In some versions, the protein exhibits a decrease in k_{cat}/Kₘ in hydrolyzing Des-Arg9-bradykinin with respect to a protein comprising an amino acid sequence 100% identical to positions 18-615 of SEQ ID NO:1.

Another aspect of the invention is directed to a recombinant polynucleotide encoding an unnatural, mutant ACE2 protein of the invention.

Another aspect of the invention is directed to a vector comprising a recombinant polynucleotide of the invention.

Another aspect of the invention is directed to recombinant cell comprising a recombinant polynucleotide of the invention.

Another aspect of the invention is directed to methods of methods of treating an ACE2-sensitive condition. The method preferably comprises administering to a subject in need thereof an unnatural, mutant ACE2 protein of the invention in an amount effective to treat the ACE2-sensitive condition.

The objects and advantages of the invention will appear more fully from the following detailed description of the preferred embodiment of the invention made in conjunction with the accompanying drawings.

### BRIEF DESCRIPTION OF THE DRAWINGS

FIG. 1. Key peptidase reactions relevant to Ang-II/ATR/MasR therapeutic axis. Red arrows denote hydrolysis reactions with known or potentially undesirable in vivo effects. Green arrows denote hydrolysis reactions with established or posited beneficial effects. 1. ACE dipeptidase removes two C-terminal residues (His-Leu) from Ang-I (DRVYIHPFHL (SEQ ID NO:3)) to yield Ang-II (DRVYIHPF (residues 1-8 of SEQ ID NO:3)). 2. ACE2 removes C-terminal Leu from Ang-I to produce Ang1-9 (DRVYIHPFH (residues 1-9 of SEQ ID NO:3)) (very slow relative to reactions 1 and 3). 3. ACE2 removes C-terminal Phe from Ang-II to produce Ang1-7 (DRVYIHP (residues 1-7 of SEQ ID NO:3)). 4. ACE2 removes C-terminal Phe from Apelin-13 (QRPRLSHKGPMPF (SEQ ID NO:4)) to yield Apelin-12 (QRPRLSHKGPMP (residues 1-12 of SEQ ID NO:4)); X denotes desirability of ACE2 variants with reduced propensity to catalyze this Apelin-13 hydrolysis.

FIG. 2. Yeast display schematic. ACE2 C-terminus linked to Aga2 native yeast surface protein on yeast cell wall. Anti-myc IgY (from chicken) used to quantify ACE2 display. Anti-myc IgY is not fluorescently labeled; fluorescent detection of myc epitope tag achieved via incubation with Alexa488-conjugated anti-chicken IgG (from goat) as secondary label (not depicted). Each yeast cell displays up to 10⁴ copies of a single ACE2 variant on its surface.

FIG. 3. Histograms for flow cytometric analysis of ACE2 display on yeast surface. One-hundred thousand yeast cells were incubated for one hour at 4° C. on a tube rotator at 18 rpm in 200 μL of PBS/0.2% BSA containing 3 μg/mL anti-myc IgY (Aves Labs, Tigard, OR). Following primary labeling yeast were washed once in PBS/0.2% BSA and rotated at 18 rpm in same buffer containing 2 μg/mL Alexa488-conjugated goat anti-chicken IgG (Jackson ImmunoResearch, West Grove, PA). Histogram X-axes denote Alexa488 fluorescence (ACE2 display); Y-axes denote number of yeast cells with a given level of fluorescence. For poorly understood biological reasons homogeneous populations of yeast carrying identical display plasmids feature 25% or greater cells (left of histograms) that do not display protein. Positive control yeast carry display plasmid for horseradish peroxidase. Histograms depict results for 1.5*10⁴ yeast cells. Data collected using a Becton-Dickinson Fortessa X-20 flow cytometer.

FIGS. 4A-4D. LC chromatograms for ACE2-displaying and negative control yeast incubated with ACE2 peptide substrates. Y-axes denote UV detector signal (mV at 214 nm). X-axes denote peptide elution time; input substrate and hydrolysis products denoted on figures. All reactions were carried out with 3*10⁵ yeast in 400 μL reaction volume with tumbling for 2.5 hours at room temperature. Horseradish peroxidase-displaying yeast (left panels) used as negative control. FIG. 4A. Reaction with 25 μM Ang-II. FIG. 4B. Reaction with 50 μM Apelin-13. FIG. 4C. Reaction with 25 μM Ang-I. FIG. 4D. Multiplex reaction containing 25 μM Ang-II, 50 μM Apelin-13 and with 25 μM Ang-I. Lysozyme, added as a carrier protein, appears in all chromatograms at elution time of 8.8 minutes.

FIG. 5. ACE2 active site residues chosen for site-directed single mutant library construction mapped onto ACE2 crystal structure (pdb entry 1R4L). For ACE2-mediated hydrolysis of the Ang-II octapeptide the ACE2 upstream S1 subsite encases Ang-II Pro7 while the downstream S1′ subsite accommodates Ang-II Phe8. Selected ACE2 residues and catalytic Zn²⁺ ion hidden from image to provide enhanced view of ACE2 substrate-binding subsites. Figure created using Pymol.

FIG. 6. Substrate hydrolysis profiles for wild type and leading ACE2 single variants from position 360, 371 and 510 site-directed mutant libraries as observed in multiplex hydrolysis assays executed with 25 μM each Ang-I, Ang-II and Apelin-13. Y-axis values denote Ang-II hydrolysis product and ratio of Ang-II:Apelin-13 hydrolysis products for ACE2 variants relative to wild type; wild type values normalized to one. Ang-II and Apelin-13 hydrolysis products quantified by LC chromatogram peak integration (UV detector mV*sec). Hydrolysis of Ang-I was not detectable under these assay conditions.

FIGS. 7A and 7B. Substrate hydrolysis profiles for wild type ACE2, parental single variants and leading ACE2 variants identified in screening of 360-clone 7/NNB 371, 510-clone 11/NNB360 and 510-clone 11/NNB 371 double mutant libraries as observed in multiplex hydrolysis assays executed with 25 μM each Ang-I, Ang-II and Apelin-13. Y-axis values denote Ang-II hydrolysis product (FIG. 7A) and ratio of Ang-II:Apelin-13 hydrolysis products (FIG. 7B) for ACE2 variants relative to wild type; wild type values normalized to one. Ang-II and Apelin-13 hydrolysis products quantified by LC chromatogram peak integration (UV detector mV*sec). Hydrolysis of Ang-I was not detectable under these assay conditions. Error bars denote standard deviations for two assays for cases in which ACE2 variant sequencing showed that replicates of a given double mutant clone had been assayed during screening.

FIG. 8. Post-purification SDS-PAGE analysis of wild type and leading mutant ACE2s. Empty vector transfection culture negative control supernatant appears in rightmost lane. Expression levels and purities for the M360P and M360P/Y510I ACE2 mutants, which had low Ang-II hydrolysis activities and are not included in this gel, were similar to those for wild type ACE2.

FIGS. 9A-9D. Initial rates plots for single substrate hydrolysis assays with respective Ang-II (FIG. 9A) and Apelin-13 (FIG. 9B) peptides. Assays contained 700 μM (Ang-II) or 1.4 nM (Apelin-13) ACE2 protein. Error bars denote standard deviations for duplicate assays; absence of error bars for some data points indicates standard deviation less than height of data point marker. Lines denote nonlinear regression data fits as calculated by GraphPad Prism software. FIGS. 9C and 9D contain rate data for lowest three substrate peptide (0.25 μM, 0.5 μM and 1 μM) concentrations assayed in FIGS. 9A and 9B, respectively.

FIGS. 10A and 10B. Comparison of mutant and wild type ACE2 Ang-II and Apelin-13 single substrate assay initial hydrolysis rates over the 0.25 μM to 10 μM peptide substrate range. Ratios are based on rate values presented in FIGS. 9A-9D and are set to one for wild type ACE2. Ang-II hydrolysis ratio (FIG. 10A) defined as mutant ACE2 hydrolysis rate divided by wild type ACE2 hydrolysis rate at a given Ang-II concentration. Ang-II to Apelin-13 specificity ratio (FIG. 10B) defined as a mutant's Ang-II hydrolysis ratio for a given Ang-II concentration multiplied by the wild type ACE2 Apelin-13 hydrolysis rate and divided by that mutant's Apelin-13 hydrolysis rate with both of these rates having been measured at an Apelin-13 concentration matching that for the Ang-II hydrolysis ratio. Error bars reflect standard deviations for duplicate measurements and are determined using error propagation as described in Materials and Methods.

FIGS. 11A and 11B. Initial rates plots for single substrate Ang-I hydrolysis assays. Assays contained 1.75 nM ACE2 protein. Error bars denote standard deviations for duplicate assays; absence of error bars for some data points indicates standard deviation less than height of data point marker. Lines denote nonlinear regression data fits as calculated by GraphPad Prism software. Highest Ang-I concentration assayed, i.e., 500 μM, was below that required to accurately estimate kinetic parameters for wild type and T371L/M510I ACE2s. FIG. 11B contains rate data for lowest four Ang-I (0.5 μM, 1 μM, 2 μM and 5 μM) concentrations assayed in FIG. 11A.

FIG. 12. LC chromatograms for Ang1-9 hydrolysis assays. Analyses correspond to assays carried out with 100 μM Ang1-9 peptide. Peaks appear for the input Ang1-9 peptide at ˜ 10.2 min). No visible peak for Ang1-9 hydrolysis product; C-terminal hydrolysis of Ang1-9 would yield Ang-II and be expected to elute from the LC column at approximately 8.8 minutes. Peaks in 2-3 minute range are Zn²⁺-EDTA complexes that form after EDTA is added to chelate the ACE2 active site Zn²⁺ ion and thus halt any ongoing substrate hydrolysis.

FIG. 13. Comparison of mutant and wild type ACE2 des-Arg9-bradykinin hydrolysis rates. Assays contained 700 μM ACE2 protein. Error bars in plots denote standard deviations for duplicate measurements.

FIGS. 14A-14D. Mutant ACE2 Ang-II hydrolysis ratios and Ang-II specificity ratios for multiplex peptide substrate hydrolysis rate assays. Ratios are set to one for wild type ACE2. Ang-II hydrolysis ratio (FIGS. 14A and 14B) defined as mutant ACE2 hydrolysis rate divided by wild type ACE2 hydrolysis rate at a given substrate concentration. Ang-II to Apelin-13 specificity ratio (FIGS. 14C and 14D) defined as a mutant's Ang-II hydrolysis ratio for a given Ang-II concentration multiplied by the wild type ACE2 Apelin-13 hydrolysis rate and divided by that mutant's Apelin-13 hydrolysis rate. Ang-I, Ang-II and Apelin-13 were present at identical concentrations in each reaction. FIGS. 14A and 14C depict results for 250 μM ACE2 reaction condition. FIGS. 14B and 14D show results for 700 μM ACE2. Error bars denote standard deviations for duplicate measurements.

FIGS. 15A and 15B. Comparison of mutant and wild type ACE2 Ang-II (FIG. 15A) and Apelin-13 (FIG. 15B) hydrolysis rates across single substrate and multiplex substrate assays carried out with peptide concentrations of 0.25 μM. Error bars denote standard deviations for duplicate measurements.

FIG. 16. Positioning of key residues M360, T371, and Y510 in ACE2 substrate binding pocket (pdb entry 1R4L). Co-crystallized ACE2 small molecule inhibitor denoted in yellow. Orange arc denotes approximate boundary between upstream S1 subsite and downstream S1′ subsite. Selected ACE2 residues and catalytic Zn²⁺ ion hidden from image to provide enhanced view of M360, T371 and Y510 ACE2 residues and small molecule inhibitor. Figure created using Pymol.

FIGS. 17A and 17B. Mutant ACE2 Ang-II hydrolysis ratios (FIG. 17A) and Ang-II specificity ratios (FIG. 17B) with 250 μM of wild type (WT) ACE2, T371L/Y510I-P235Q ACE2, and T371L/Y510I ACE2 using the same methods employed for FIGS. 14A-14D.

### DETAILED DESCRIPTION OF THE INVENTION

One aspect of the invention is directed to mutant ACE2 proteins. The mutant ACE2 proteins comprise an amino acid sequence with at least one mutation with respect to a native amino acid sequence. “Native amino acid sequence” refers to the full amino acid sequence, or any contiguous portion thereof, of any protein found in nature.

An exemplary protein with a native amino acid sequence is the human ACE2 protein, which has the amino acid sequence of SEQ ID NO:1:

(SEQ ID NO: 1)
MSSSSWLLLSLVAVTAAQSTIEEQAKTFLDKENHEAEDLFYQSSLASWNY
NTNITEENVQNMNNAGDKWSAFLKEQSTLAQMYPLQEIQNLTVKLQLQAL
QQNGSSVLSEDKSKRLNTILNTMSTIYSTGKVCNPDNPQECLLLEPGLNE
IMANSLDYNERLWAWESWRSEVGKOLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDELTAHHEMGHIQYDMAYAAQPELLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDONK
NSFVGWSTDWSPYADQSIKVRISLKSALGDRAYEWNDNEMYLFRSSVAYA
MRQYFLKVKNQMILFGEEDVRVANLKPRISENFFVTAPKNVSDIIPRTEV
EKAIRMSRSRINDAFRLNDNSLEFLGIQPTLGPPNQPPVSIWLIVFGVVM
GVIVVGIVILIFTGIRDRKKKNKARSGENPYASIDISKGENNPGFQNTDD
VQTSF

This exemplary human ACE2 protein comprises a signal peptide (positions 1-17), an extracellular domain (positions 18-740), a helical transmembrane domain (positions 741-761), and a cytoplasmic domain (positions 762-805). Positions 18-615 within the extracellular domain comprise a catalytic domain. The catalytic domain can be employed without the other portions of the protein as an active, soluble form of the protein. The amino acid sequence of any of these parts of the human ACE2 protein, whether in combination with or in isolation of the others, is understood herein to constitute a native amino acid sequence.

An exemplary coding sequence for the human ACE2 protein represented by SEQ ID NO:1 is SEQ ID NO:2:

(SEQ ID NO: 2)
ATGTCAAGCTCTTCCTGGCTCCTTCTCAGCCTTGTTGCTGTAACTGCTGC
TCAGTCCACCATTGAGGAACAGGCCAAGACATTTTTGGACAAGTTTAACC
ACGAAGCCGAAGACCTGTTCTATCAAAGTTCACTTGCTTCTTGGAATTAT
AACACCAATATTACTGAAGAGAATGTCCAAAACATGAATAATGCTGGGGA
CAAATGGTCTGCCTTTTTAAAGGAACAGTCCACACTTGCCCAAATGTATC
CACTACAAGAAATTCAGAATCTCACAGTCAAGCTTCAGCTGCAGGCTCTT
CAGCAAAATGGGTCTTCAGTGCTCTCAGAAGACAAGAGCAAACGGTTGAA
CACAATTCTAAATACAATGAGCACCATCTACAGTACTGGAAAAGTTTGTA
ACCCAGATAATCCACAAGAATGCTTATTACTTGAACCAGGTTTGAATGAA
ATAATGGCAAACAGTTTAGACTACAATGAGAGGCTCTGGGCTTGGGAAAG
CTGGAGATCTGAGGTCGGCAAGCAGCTGAGGCCATTATATGAAGAGTATG
TGGTCTTGAAAAATGAGATGGCAAGAGCAAATCATTATGAGGACTATGGG
GATTATTGGAGAGGAGACTATGAAGTAAATGGGGTAGATGGCTATGACTA
CAGCCGCGGCCAGTTGATTGAAGATGTGGAACATACCTTTGAAGAGATTA
AACCATTATATGAACATCTTCATGCCTATGTGAGGGCAAAGTTGATGAAT
GCCTATCCTTCCTATATCAGTCCAATTGGATGCCTCCCTGCTCATTTGCT
TGGTGATATGTGGGGTAGATTTTGGACAAATCTGTACTCTTTGACAGTTC
CCTTTGGACAGAAACCAAACATAGATGTTACTGATGCAATGGTGGACCAG
GCCTGGGATGCACAGAGAATATTCAAGGAGGCCGAGAAGTTCTTTGTATC
TGTTGGTCTTCCTAATATGACTCAAGGATTCTGGGAAAATTCCATGCTAA
CGGACCCAGGAAATGTTCAGAAAGCAGTCTGCCATCCCACAGCTTGGGAC
CTGGGGAAGGGCGACTTCAGGATCCTTATGTGCACAAAGGTGACAATGGA
CGACTTCCTGACAGCTCATCATGAGATGGGGCATATCCAGTATGATATGG
CATATGCTGCACAACCTTTTCTGCTAAGAAATGGAGCTAATGAAGGATTC
CATGAAGCTGTTGGGGAAATCATGTCACTTTCTGCAGCCACACCTAAGCA
TTTAAAATCCATTGGTCTTCTGTCACCCGATTTTCAAGAAGACAATGAAA
CAGAAATAAACTTCCTGCTCAAACAAGCACTCACGATTGTTGGGACTCTG
CCATTTACTTACATGTTAGAGAAGTGGAGGTGGATGGTCTTTAAAGGGGA
AATTCCCAAAGACCAGTGGATGAAAAAGTGGTGGGAGATGAAGCGAGAGA
TAGTTGGGGTGGTGGAACCTGTGCCCCATGATGAAACATACTGTGACCCC
GCATCTCTGTTCCATGTTTCTAATGATTACTCATTCATTCGATATTACAC
AAGGACCCTTTACCAATTCCAGTTTCAAGAAGCACTTTGTCAAGCAGCTA
AACATGAAGGCCCTCTGCACAAATGTGACATCTCAAACTCTACAGAAGCT
GGACAGAAACTGTTCAATATGCTGAGGCTTGGAAAATCAGAACCCTGGAC
CCTAGCATTGGAAAATGTTGTAGGAGCAAAGAACATGAATGTAAGGCCAC
TGCTCAACTACTTTGAGCCCTTATTTACCTGGCTGAAAGACCAGAACAAG
AATTCTTTTGTGGGATGGAGTACCGACTGGAGTCCATATGCAGACCAAAG
CATCAAAGTGAGGATAAGCCTAAAATCAGCTCTTGGAGATAAAGCATATG
AATGGAACGACAATGAAATGTACCTGTTCCGATCATCTGTTGCATATGCT
ATGAGGCAGTACTTTTTAAAAGTAAAAAATCAGATGATTCTTTTTGGGGA
GGAGGATGTGCGAGTGGCTAATTTGAAACCAAGAATCTCCTTTAATTTCT
TTGTCACTGCACCTAAAAATGTGTCTGATATCATTCCTAGAACTGAAGTT
GAAAAGGCCATCAGGATGTCCCGGAGCCGTATCAATGATGCTTTCCGTCT
GAATGACAACAGCCTAGAGTTTCTGGGGATACAGCCAACACTTGGACCTC
CTAACCAGCCCCCTGTTTCCATATGGCTGATTGTTTTTGGAGTTGTGATG
GGAGTGATAGTGGTTGGCATTGTCATCCTGATCTTCACTGGGATCAGAGA
TCGGAAGAAGAAAAATAAAGCAAGAAGTGGAGAAAATCCTTATGCCTCCA
TCGATATTAGCAAAGGAGAAAATAATCCAGGATTCCAAAACACTGATGAT
GTTCAGACCTCCTTTTAG

The mutant ACE2 proteins of the invention may comprise an amino acid sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to positions 1-805, positions 18-805, positions 18-740, or positions 18-615 of SEQ ID NO:1. Preferred mutant ACE2 proteins of the invention comprise an amino acid sequence at least about 90%, at least about 91%, at least about 92%, at least about 93%, at least about 94%, at least about 95%, at least about 96%, at least about 97%, at least about 98%, or at least about 99% identical to positions 18-615 of SEQ ID NO:1.

The mutant ACE2 proteins of the invention may have one or more substitutions at positions corresponding to particular positions of SEQ ID NO: 1. For example, the mutant ACE2 proteins may comprise one or more of: a residue other than glutamate at a position corresponding to position 145 of SEQ ID NO:1; a residue other than asparagine at a position corresponding to position 149 of SEQ ID NO:1; a residue other than arginine at a position corresponding to position 273 of SEQ ID NO:1; a residue other than threonine at a position corresponding to position 347 of SEQ ID NO:1; a residue other than methionine at a position corresponding to position 360 of SEQ ID NO:1; a residue other than lysine at a position corresponding to position 363 of SEQ ID NO:1; a residue other than threonine at a position corresponding to position 371 of SEQ ID NO:1; a residue other than phenylalanine at a position corresponding to position 504 of SEQ ID NO:1; a residue other than tyrosine at a position corresponding to position 510 of SEQ ID NO:1; and a residue other than arginine at a position corresponding to position 514 of SEQ ID NO:1.

The mutant ACE2 proteins of the invention may comprise one or more of a residue other than glutamate at a position corresponding to position 145 of SEQ ID NO:1; a residue other than asparagine at a position corresponding to position 149 of SEQ ID NO: 1; a residue other than methionine at a position corresponding to position 360 of SEQ ID NO: 1; a residue other than lysine at a position corresponding to position 363 of SEQ ID NO:1; a residue other than threonine at a position corresponding to position 371 of SEQ ID NO: 1; and a residue other than tyrosine at a position corresponding to position 510 of SEQ ID NO:1.

The mutant ACE2 proteins of the invention may comprise one or more of a residue other than methionine at a position corresponding to position 360 of SEQ ID NO:1; a residue other than threonine at a position corresponding to position 371 of SEQ ID NO: 1; and a residue other than tyrosine at a position corresponding to position 510 of SEQ ID NO:1.

The mutant ACE2 proteins of the invention may comprise one or more of leucine, proline, or a conservative variant of leucine or proline at a position corresponding to position 360 of SEQ ID NO: 1; alanine, aspartate, leucine, phenylalanine, serine, or a conservative variant or alanine, aspartate, leucine, phenylalanine, or serine, at a position corresponding to position 371 of SEQ ID NO:1; and isoleucine, valine, or a conservative variant of isoleucine or valine at a position corresponding to position 510 of SEQ ID NO:1.

The mutant ACE2 proteins of the invention may comprise two or more of a residue other than methionine at a position corresponding to position 360 of SEQ ID NO:1; a residue other than threonine at a position corresponding to position 371 of SEQ ID NO: 1; and a residue other than tyrosine at a position corresponding to position 510 of SEQ ID NO: 1.

The mutant ACE2 proteins of the invention may comprise two or more of: leucine, proline, or a conservative variant of leucine or proline at a position corresponding to position 360 of SEQ ID NO:1; alanine, aspartate, leucine, phenylalanine, serine, or a conservative variant or alanine, aspartate, leucine, phenylalanine, or serine, at a position corresponding to position 371 of SEQ ID NO:1; and isoleucine, valine, or a conservative variant of isoleucine or valine at a position corresponding to position 510 of SEQ ID NO:1.

The mutant ACE2 proteins of the invention may comprise isoleucine, valine, or a conservative variant of isoleucine or valine at a position corresponding to position 510 of SEQ ID NO:1.

The mutant ACE2 proteins of the invention may comprise one or more of leucine, proline, or a conservative variant of leucine or proline at a position corresponding to position 360 of SEQ ID NO:1; and alanine, aspartate, leucine, phenylalanine, serine, or a conservative variant or alanine, aspartate, leucine, phenylalanine, or serine, at a position corresponding to position 371 of SEQ ID NO:1.

The mutant ACE2 proteins of the invention may comprise isoleucine at a position corresponding to position 510 of SEQ ID NO:1.

The mutant ACE2 proteins of the invention may comprise isoleucine at a position corresponding to position 510 of SEQ ID NO:1 in addition to leucine at a position corresponding to position 360 of SEQ ID NO:1, leucine at a position corresponding to position 371 of SEQ ID NO: 1, or leucine at a position corresponding to position 360 of SEQ ID NO:1 and leucine at a position corresponding to position 371 of SEQ ID NO:1.

In some versions, the mutant ACE2 proteins of the invention are soluble (i.e., not membrane bound). In some versions, the mutant ACE2 proteins of the invention lack an amino acid sequence at least about 90%, at least about 91%, at least about 92%, at least about 93%, at least about 94%, at least about 95%, at least about 96%, at least about 97%, at least about 98%, at least about 99%, or 100% identical to 1-17 of SEQ ID NO:1. In some versions, the mutant ACE2 proteins of the invention lack an amino acid sequence at least about 90%, at least about 91%, at least about 92%, at least about 93%, at least about 94%, at least about 95%, at least about 96%, at least about 97%, at least about 98%, at least about 99%, or 100% identical to 616-740 of SEQ ID NO:1. In some versions, the mutant ACE2 proteins of the invention lack an amino acid sequence at least about 90%, at least about 91%, at least about 92%, at least about 93%, at least about 94%, at least about 95%, at least about 96%, at least about 97%, at least about 98%, at least about 99%, or 100% identical to 740-761 of SEQ ID NO:1. In some versions, the mutant ACE2 proteins of the invention lack an amino acid sequence at least about 90%, at least about 91%, at least about 92%, at least about 93%, at least about 94%, at least about 95%, at least about 96%, at least about 97%, at least about 98%, at least about 99%, or 100% identical to 762-805 of SEQ ID NO:1. In some versions, the mutant ACE2 proteins of the invention lack an amino acid sequence at least about 90%, at least about 91%, at least about 92%, at least about 93%, at least about 94%, at least about 95%, at least about 96%, at least about 97%, at least about 98%, at least about 99%, or 100% identical to 1-17 of SEQ ID NO:1 and also lack an amino acid sequence at least about 90%, at least about 91%, at least about 92%, at least about 93%, at least about 94%, at least about 95%, at least about 96%, at least about 97%, at least about 98%, at least about 99%, or 100% identical to 616-805 of SEQ ID NO:1. In some versions, the mutant ACE2 proteins of the invention lack a signal peptide, such as the signal peptide of a human ACE2 protein. In some versions, the mutant ACE2 proteins of the invention lack a helical transmembrane domain, such as the helical transmembrane domain of a human ACE2 protein. In some versions, the mutant ACE2 proteins of the invention lack a cytoplasmic domain, such as the cytoplasmic domain of a human ACE2 protein.

In some versions, the mutant ACE2 proteins of the invention comprise an amino acid sequence at least about 90%, at least about 91%, at least about 92%, at least about 93%, at least about 94%, at least about 95%, at least about 96%, at least about 97%, at least about 98%, or at least about 99% identical to positions 18-615 of SEQ ID NO:1 fused to a heterologous amino acid sequence. The heterologous amino acid sequence can constitute a primary structure of any of a number of heterologous domains. Exemplary domains include linkers, affinity tags, or other catalytically active domains, among others. Linkers employed to fuse two heterologous polypeptides or domains to generate fusion proteins are well known in the art. See, e.g., U.S. Pat. Nos. 5,525,491, 6,274,331, 6,479,626, 10,526,379, 10,752,965, and 11,123,438, among others. Exemplary linkers include linkers comprising glycine and serine, such as a -G-S- linker or a -G-S-G- linker. Exemplary linker lengths can be from 1-20 residues in length, such as from 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 residues in length. Exemplary affinity tags include the His tag, the Strep II tag, the T7 tag, the FLAG tag, the S tag, the HA tag, the c-Myc tag, the dihydrofolate reductase (DHFR) tag, the chitin binding domain tag, the calmodulin binding domain tag, and the cellulose binding domain tag. The sequences of each of these tags are well-known in the art. Preferred affinity tags are those smaller than about 20 amino acids, such as the His tag, the Strep II tag, the T7 tag, the FLAG tag, the S tag, the HA tag, the c-Myc tag. Domains fused to the amino acid sequence of at least about 90%, at least about 91%, at least about 92%, at least about 93%, at least about 94%, at least about 95%, at least about 96%, at least about 97%, at least about 98%, or at least about 99% identical to positions 18-615 of SEQ ID NO:1 can be fused either directly or indirectly via a linker and, in various versions, can have a size less than 500 amino acids in length, less than 475 amino acids in length, less than 450 amino acids in length, less than 425 amino acids in length, less than 400 amino acids in length, less than 375 amino acids in length, less than 350 amino acids in length, less than 325 amino acids in length, less than 300 amino acids in length, less than 275 amino acids in length, less than 250 amino acids in length, less than 225 amino acids in length, less than 200 amino acids in length, less than 175 amino acids in length, less than 150 amino acids in length, less than 125 amino acids in length, less than 100 amino acids in length, less than 75 amino acids in length, less than 50 amino acids in length, or less than 25 amino acids in length.

The mutant ACE2 proteins of the invention may have mutations with respect to a corresponding ACE2 protein. “Corresponding ACE2 protein” refers to a protein comprising or consisting of a corresponding amino acid sequence. A corresponding sequence, such as a corresponding amino acid sequence or a corresponding nucleic acid sequence, is a sequence that aligns to the sequence of a given mutant ACE2 protein, or any portion thereof, using bioinformatic techniques, for example, using the methods described herein for preparing a sequence alignment. The corresponding sequence preferably has at least 90% identity, at least 91% identity, at least 92% identity, at least 93% identity, at least 94% identity, at least 95% identity, at least 96% identity, at least 97% identity, at least 98% identity, at least 99% identity, or 100% identity to the mutant ACE2 sequence over the aligned portions of each sequence. The corresponding amino acid sequence is preferably a native amino acid sequence. An exemplary corresponding amino acid sequence is the sequence of positions 18-615 of SEQ ID NO:1. An exemplary corresponding ACE2 protein is a protein consisting of or comprising the sequence of positions 18-615 of SEQ ID NO:1.

The mutant ACE2 proteins of the invention preferably have at least one altered property compared to the properties of a corresponding ACE2 protein. The altered property preferably comprises an enhancement of an aspect of ACE2 activity. The altered property may be exhibited in vitro, in vivo, or both in vitro and in vivo. The altered property may include increased activity, increased specificity, or increased activity and specificity in hydrolyzing a particular substrate, such as Angiotensin II (DRVYIFPFH (residues 1-9 of SEQ ID NO:3)). In some versions, the altered property comprises an increase in k_{cat} in hydrolyzing Angiotensin II. In some versions, the altered property comprises a decrease in Kₘ in hydrolyzing Angiotensin II. In some versions, the altered property comprises a decrease in Kₘ in hydrolyzing Angiotensin II. In some versions, the altered property comprises an increase in kea/Kₘ in hydrolyzing Angiotensin II. In some versions, the altered property comprises an increase in Angiotensin II:Apelin-13 hydrolysis ratio. In some versions, the altered property comprises a decrease in k_{cat}/Kₘ in hydrolyzing Apelin-13 (QRPRLSHKGPMPF (SEQ ID NO:4)). In some versions, the altered property comprises an increase in k_{cat}/Kₘ in hydrolyzing Angiotensin I. In some versions, the altered property comprises a decrease in k_{cat}/Kₘ in hydrolyzing Des-Arg9-bradykinin (RPPGFSPF (residues 1-8 of SEQ ID NO:5)). In some versions, the altered property comprises a decrease in k_{cat}/Kₘ in hydrolyzing any one or more of Angiotensin 1-9 (DRVYIHPFH (residues 1-9 of SEQ ID NO:3)), Angiotensin 1-7 (DRVYIHP (residues 1-7 of SEQ ID NO:3)), Angiotensin 1-5 (DRVYI (residues 1-5 of SEQ ID NO:3)), Apelin-36 (C terminus comprising QRPRLSHKGPMPF (SEQ ID NO:4)), Bradykinin (RPPGFSPFR (SEQ ID NO:5)), Lys-des-Arg9-bradykinin (KRPPGFSPF (SEQ ID NO:6)), Bradykinin fragment 1-7 (RPPGFSP (residues 1-7 of SEQ ID NO:5)), β-Casomorphin (YPFVEPI (SEQ ID NO:7)), Neocasomorphin (YPVEPI (SEQ ID NO:8)), Dynorphin A 1-13 (YGGFLRRIRPKLK (SEQ ID NO:9)), Ghrelin (C terminus comprising ESKKPPAKLQPR (SEQ ID NO:10)), Neurotensin 1-8 ((pE)LYENKPR (residues 1-8 of SEQ ID NO:11) (pE is pyroglutamic acid)), and Neurotensin ((pE)LYENKPRRPYIL (SEQ ID NO:11) (pE is pyroglutamic acid)). In some versions, the corresponding ACE2 protein is a protein comprising an amino acid sequence 100% identical to positions 18-615 of SEQ ID NO:1. In some versions, the corresponding ACE2 protein is a protein consisting of an amino acid sequence 100% identical to positions 18-615 of SEQ ID NO:1. In some versions, the corresponding ACE2 protein is a protein comprising an amino acid sequence identical to the amino acid sequence of a mutant ACE2 protein of the invention, except for any one or more of the mutations specified herein for a mutant ACE2 protein of the invention. In some versions, the corresponding ACE2 protein is a protein consisting of an amino acid sequence identical to the amino acid sequence of a mutant ACE2 protein of the invention, except for any one or more of the mutations specified herein for a mutant ACE2 protein of the invention.

Another aspect of the invention is a polynucleotide (or a gene) encoding a mutant ACE2 protein of the invention. Another aspect of the invention is a vector comprising the polynucleotide (or the gene) according to the invention. Vectors of the invention can be transformed into suitable host cells to produce recombinant host cells.

Another aspect of the invention is a recombinant host cell comprising a polynucleotide encoding a mutant ACE2 protein of the invention. In some versions, known genomic alteration or modification techniques can be employed to alter or modify the endogenous ACE2 protein of the host cell, effectuating one or more of the aforementioned mutations, such that at least one of the mutant endogenous ACE2 proteins has at least one altered property. In other versions, the recombinant host cell is engineered to include a plasmid comprising a polynucleotide encoding a mutant ACE2 protein. In yet other versions, the recombinant host cell is engineered to include the polynucleotide encoding the mutant ACE2 protein integrated into the chromosome of the host cell.

The recombinant host cell of the invention can be selected from any cell capable of expressing a recombinant gene construct, and can be selected from a microbial, plant or animal cell. In a particular embodiment, the host cell is bacterial, cyanobacterial, fungal, yeast, algal, human or mammalian in origin. In a particular embodiment, the host cell is selected from any of Gram positive bacterial species such as Actinomycetes; Bacillaceae, including Bacillus alkalophilus, Bacillus subtilis, Bacillus licheniformis, Bacillus lentus, Bacillus brevis, Bacillus stearothermophilus, Bacillus alkalophilus, Bacillus amyloliquefaciens, Bacillus coagulans, Bacillus circulans, Bacillus lautus, Bacillus megaterium, B. thuringiensis; Brevibacteria sp., including Brevibacterium flavum, Brevibacterium lactofermentum, Brevibacterium ammoniagenes, Brevibacterium butanicum, Brevibacterium divaricatum, Brevibacterium healii, Brevibacterium ketoglutamicum, Brevibacterium ketosoreductum, Brevibacterium lactofermentum, Brevibacterium linens, Brevibacterium paraffinolyticum; Corynebacterium spp. such as C. glutamicum and C. melassecola, Corynebacterium herculis, Corynebacterium lilium, Corynebactertium acetoacidophilum, Corynebacterium acetoglutamicum, Corynebacterium acetophilum, Corynebacterium ammoniagenes, Corynebacterium fujiokense, Corynebacterium nitrilophilus; or lactic acid bacterial species including Lactococcus spp. such as Lactococcus lactis; Lactobacillus spp. including Lactobacillus reuteri; Leuconostoc spp.; Pediococcus spp.; Serratia spp. such as Serratia marcescens; Streptomyces species, such as Streptomyces lividans, Streptomyces murinus, S. coelicolor and Streptococcus spp. Alternatively, strains of a Gram negative bacterial species belonging to Enterobacteriaceae including E. coli, Cellulomonas spp.; or to Pseudomonadaceae including Pseudomonas aeruginosa, Pseudomonas alcaligenes, Pseudomonas fluorescens, Pseudomonas putida, Pseudomonas syringae and Burkholderia cepacia, Salmonella sp., Stenotrophomonas spp., and Stenotrophomonas maltophilia. Oleaginous microorganisms such as Rhodococcus spp, Rhodococcus opacus, Ralstonia spp., and Acetinobacter spp. are useful as well. Furthermore, yeasts and filamentous fungal strains can be useful host cells, including Absidia spp.; Acremonium spp.; Agaricus spp.; Anaeromyces spp.; Aspergillus spp., including A. aculeatus, A. awamori, A. flavus, A. foetidus, A. fumaricus, A. fumigatus, A. nidulans, A. niger, A. oryzae, A. terreus; A. tubingensis and A. versicolor; Aeurobasidium spp.; Cephalosporum spp.; Chaetomium spp.; Coprinus spp.; Dactyllum spp.; Fusarium spp., including F. conglomerans, F. decemcellulare, F. javanicum, F. lini, F. oxysporum and F. solani; Gliocladium spp.; Kluyveromyces sp.; Hansenula sp.; Humicola spp., including H. insolens and H. lanuginosa; Hypocrea spp.; Mucor spp.; Neurospora spp., including N. crassa and N. sitophila; Neocallimastix spp.; Orpinomyces spp.; Penicillium spp.; Phanerochaete spp.; Phlebia spp.; Pichia sp.; Piromyces spp.; Rhizopus spp.; Rhizomucor species such as Rhizomucor miehei; Saccaromyces species such as S. cerevisiae, S. pastorianus, S. eubayanus, and S. fragilis; Schizophyllum spp.; Schizosaccharomyces such as, for example, S. pombe species; chytalidium sp., Sulpholobus sp., Thermoplasma sp., Thermomyces sp.; Trametes spp.; Trichoderma spp., including T. reesei, T. reesei (longibrachiatum) and T. viride; Yarrowinia sp.; and Zygorhynchus spp and in particular include oleaginous yeast just Phafia spp., Rhorosporidium toruloides Y4, Rhodotorula Glutinis and Candida 107.

In some versions of the invention, genes encoding mutant ACE2 proteins and/or other recombinantly expressed genes in a recombinant host cell are modified to optimize at least one codon for expression in the recombinant host cell.

In some versions of the invention, a method is provided wherein the recombinant host cell according to the invention is cultured under conditions that permit expression or overexpression of a mutant ACE2 protein of the invention. The mutant ACE2 protein can be recovered, and more preferably substantially purified, after the host cell is harvested and/or lysed.

Another aspect of the invention is directed to a pharmaceutical composition comprising a mutant ACE2 protein, a recombinant polynucleotide, or a vector, of the invention and a pharmaceutically acceptable carrier or diluent(s). Such pharmaceutical compositions may further comprise pharmaceutically acceptable salts (and salts of the mutant ACE2 proteins of the invention) and optionally additional buffers, tonicity adjusting agents, pharmaceutically acceptable vehicles, and/or stabilizers. Pharmaceutically acceptable carriers or diluents are used to improve the tolerability of the composition and allow better solubility and better bioavailability of the active ingredients. Examples include emulsifiers, thickeners, redox components, starch, alcohol solutions, polyethylene glycol or lipids. The choice of suitable pharmaceutical carriers or diluents depends greatly on how the composition is to be administered. Liquid or solid carriers or diluents may be used for oral administration; whereas liquid compositions are preferably used for injections or infusions.

The pH of the pharmaceutical compositions of the invention may be adjusted to correspond to physiological pH by means of buffers, and fluctuations in pH may also be buffered. Buffers include, but are not limited to, for example a phosphate buffer or a MES (2-(N-morpholino)ethanesulfonic acid) buffer. In one embodiment, the pH of the pharmaceutical composition is between 4 and 10, such as between 5 and 9.5, such as between 6 and 9. Tonicity adjusting agents are used to adjust the osmolarity of the composition and may contain ionic substances, such as inorganic salts, e.g., NaCl, or nonionic substances, such as glycerol or carbohydrates.

The pharmaceutical compositions of the invention are suitably prepared for systemic, topical, oral or intranasal administration. These modes of administration of the pharmaceutical composition allow a rapid and uncomplicated uptake of active substance. For example, for oral administration, solid and/or liquid medications may be administered directly, or alternatively may be dissolved and/or diluted prior to administration. In one embodiment, the pharmaceutical compositions are liquid, in particular aqueous compositions.

The pharmaceutical compositions of the invention may be suitably prepared for intravenous, intra-arterial, intramuscular, intravascular, intraperitoneal, or subcutaneous administration. For example, injections or infusions, such as into the bloodstream, may be used. Administration directly into the bloodstream has the advantage that the active substance(s) of the composition are distributed throughout the entire body for systemic therapy, and rapidly reach the target tissue(s). In one embodiment, the pharmaceutical composition is prepared for intravenous administration, and may be in liquid form.

Another aspect of the invention includes methods of treating an ACE2-sensitive condition, comprising administering to a subject in need thereof a mutant ACE2 protein of the invention in an amount effective to treat the ACE2-sensitive condition. The ACE2 protein can be administered by delivering an already expressed ACE2 protein to the subject or delivering a polynucleotide, gene, or vector of the invention that expresses the ACE2 protein in sito (in vivo). Another aspect of the invention includes a mutant ACE2 protein, polynucleotide, gene, or vector of the invention for use in therapy, such as for the treatment of an ACE2-sensitive condition. Another aspect of the invention includes the use of a mutant ACE2 protein, polynucleotide, gene, or vector of the invention in the manufacture of a medicament for the treatment of an ACE2-sensitive condition.

“Treat,” “treating,” and the like, such as in the phrase “effective to treat the condition,” refers to the full or partial amelioration or prophylaxis of the condition. These terms may encompass reducing symptoms, ameliorating symptoms, alleviating symptoms, reducing the severity of symptoms, reducing the incidence of the condition, diminishing the extent of the condition, stabilizing (i.e., not worsening) the state of the condition, delaying onset of the condition, slowing progression of the condition, inducing remission (whether partial or total) of the condition, or any other change in the condition that improves the therapeutic outcome in the subject. Treating can include eliciting a clinically significant response without excessive levels of side effects.

Subjects treated in the methods of the invention include animals, such as humans and non-human vertebrates, including wild, domestic, and farm animals or mammals.

“Condition” refers to a disorder, disease, or any symptom of a disorder or disease.

The administering may comprise parenteral, systemic, topical, oral, or intranasal administration. The parenteral administration may comprise injection or infusion, such as into the bloodstream or other tissues. The parenteral administration may comprise intravenous, intra-arterial, intramuscular, intravascular, intraperitoneal, or subcutaneous administration, such as via injection or infusion.

“ACE2-sensitive condition” refers to a condition that is treatable with ACE2 activity (i.e., activity as defined under Enzyme Commission Number: 3.4.17.23).

Exemplary ACE2-sensitive conditions include heart disease and related conditions. See, e.g., US2010311822A1, incorporated herein by reference. Aspects of heart disease and conditions relating thereto include hypertension (including high blood pressure), congestive heart failure, chronic heart failure, acute heart failure, contractile heart failure, myocardial infarction, and arteriosclerosis.

Exemplary ACE2-sensitive conditions include kidney disease and related conditions. See, e.g., US2010311822A1, incorporated herein by reference. Aspects of kidney disease and conditions relating thereto include renal fibrosis, chronic renal failure, acute renal failure, chronic renal failure, acute kidney injury, polycystic kidney disease (PKD), and renal fibrosis.

Exemplary ACE2-sensitive conditions include lung disease and related conditions. See, e.g., US2010311822A1, incorporated herein by reference. Aspects of lung disease and conditions relating thereto include acute respiratory distress syndrome (ARDS), acute lung injury (ALI), chronic obstructive pulmonary disease (COPD), pulmonary hypertension, pneumonia, asthma, chronic bronchitis, pulmonary emphysema, cystic fibrosis, interstitial lung disease, primary pulmonary hypertension, pulmonary embolism, pulmonary sarcoidosis, tuberculosis, lung cancers, oedema of the lung, and pulmonary hypertonia.

Another exemplary ACE2-sensitive condition includes multi-organ dysfunction syndrome.

Another exemplary ACE2-sensitive condition includes inflammation. See, e.g., US2011020315A1, incorporated herein by reference. The inflammation is can be a local inflammation of a tissue or organ and/or a systemic inflammation. Based on the general mechanism, it is possible to treat both chronic and acute inflammations. In particular, the inflammation may include, but is not limited to, rheumatism, sepsis, osteoarthritis, rheumatoid arthritis, systemic lupus erythematosus, scleroderma or mixed connective tissue disease. These conditions may be caused by mechanical or chemical cellular damage or tissue damage or wounds; infections, in particular pathogens such as viruses, bacteria, or fungi; by implants including organ implants; and/or by medications. In one embodiment, the inflammation is caused by an infection.

Another exemplary ACE2-sensitive condition includes Alzheimer's Disease.

Other exemplary ACE2-sensitive conditions autoimmune diseases, including autoimmune diseases involving inflammation. Exemplary autoimmune diseases include an anti-glomerular basal membrane disease, autoimmune diseases of the nervous system, systemic lupus erythematosus (SLE), Addison's disease, an antiphospholipid syndrome, an IgA glomerulonephritis, a Goodpasture syndrome, a Lambert-Eaton myasthenic syndrome, idiopathic purpura, an autoimmune thyroiditis, a rheumatoid arthritis, an insulin-dependent diabetes mellitus, an pemphigus, an autoimmune hemolytic anemia, a dermatitis herpetiformis Durhing, a membranous glomerulonephritis, a Graves disease, a sympathetic ophthalmia, autoimmune polyendocrinopathies, multiple sclerosis, inflammatory bowel disease, and/or Reiter's disease.

Other exemplary ACE2-sensitive conditions include fibrosis. See, e.g., US2010316624A1, incorporated herein by reference. The fibrosis can be a local fibrosis of a tissue or organ. Such organ-specific fibroses include, but are not limited to, hepatic fibroses, pulmonary fibroses, connective tissue fibroses, in particular fibrosis of the muscle septa, renal fibrosis, and fibrosis of the skin, e.g., in combination with an inflammation-scleroderma. The fibrosis may be a fibrosis of an internal organ, e.g., the liver, kidneys, lungs, heart, stomach, intestines, pancreas, glands, muscles, cartilage, tendons, ligaments or joints. Cystic fibrosis or rheumatic fibrosis is a type of fibrosis. In another embodiment, the fibrosis occurs concurrently with inflammation, e.g., hepatitis (inflammatory liver disease). In another embodiment, the fibrosis is associated with organ transplantation. The fibrosis may be attributed to an excessive deposit of the components of the extracellular matrix, in particular proteins such as collagen. Collagen is a structural protein of the connective tissue, in particular the extracellular matrix. The formation of collagen, in particular in combination with the SMA (smooth muscle actin) marker correlates directly with the progression of fibrosis.

A particular exemplary ACE2-sensitive condition is fibrosis, such as chronic fibrosis. In particular, the chronic fibrosis may be caused by mechanical or chemical cell or tissue damage or wounds, cancer or tumors, infections, in particular pathogens such as viruses, bacteria or fungi, by implants, including organ implants as well as medications. Infections may be organ-specific, for example, such as hepatitis virus infection, in particular due to HCV. Other fibrotic diseases include, for example, primary or secondary fibroses, in particular fibroses caused by an autoimmune response, Ormond's disease (retroperitoneal fibrosis).

Another exemplary ACE2-sensitive condition is liver disease. See, e.g., US2010316624A1, incorporated herein by reference. In some embodiments, the fibrosis or liver disease occurs concurrently with inflammation, e.g., hepatitis (inflammatory liver disease).

Other ACE2-sensitive conditions include tumor diseases and cancers. See, e.g., US2011033524A1, incorporated herein by reference. Preferably the tumor diseases are selected from a tumor disease of the reproductive tract, such as ovarian cancer, testicular cancer, prostate cancer, or breast cancer; tumor diseases of the digestive tract, such as stomach cancer, intestinal cancer, rectum carcinoma, pancreatic cancer, esophagus cancer; liver cancer; kidney cancer; lung cancer; melanomas; and/or neuroblastomas.

Another exemplary ACE2-sensitive condition is diabetes and conditions relating thereto, such as diabetic nephropathy.

Other ACE2-sensitive conditions include respiratory infections. Exemplary respiratory infections include respiratory infections with viruses (e.g., influenza, coronaviridae, such as SARS-CoV-2), bacteria, mycoplasma, or fungi. Another ACE2-sensitive condition includes COVID-19.

Throughout the specification, a reference may be made using an abbreviation of a gene name or a protein name, but it is understood that such an abbreviated gene or protein name represents the genus of genes or proteins, respectively. Such gene names include all genes encoding the same protein and homologous proteins having the same physiological function. Protein names include all proteins that have the same activity (e.g., that catalyze the same fundamental chemical reaction).

Unless otherwise indicated, the accession numbers referenced herein are derived from the NCBI database (National Center for Biotechnology Information) maintained by the National Institute of Health, U.S.A.

EC numbers are established by the Nomenclature Committee of the International Union of Biochemistry and Molecular Biology (NC-IUBMB) (available at www.chem.qmul/ac/uk/iubmb/enzyme/). The EC numbers referenced herein are derived from the KEGG Ligand database, maintained by the Kyoto Encyclopedia of Genes and Genomics, sponsored in part by the University of Tokyo.

The term “ACE2 protein,” including variations thereof such as “mutant ACE2 protein,” refers to an enzyme that has activity as defined under Enzyme Commission Number: 3.4.17.23.

The term “altered property” refers to a modification in one or more properties of a mutant polynucleotide or mutant protein with reference to a corresponding polynucleotide or precursor protein.

The term “alignment” refers to a method of comparing two or more polynucleotides or polypeptide sequences for the purpose of determining their relationship to each other. Alignments are typically performed by computer programs that apply various algorithms, however it is also possible to perform an alignment by hand. Alignment programs typically iterate through potential alignments of sequences and score the alignments using substitution tables, employing a variety of strategies to reach a potential optimal alignment score. Commonly-used alignment algorithms include, but are not limited to, CLUSTALW, (see, Thompson J. D., Higgins D. G., Gibson T. J., CLUSTAL W: improving the sensitivity of progressive multiple sequence alignment through sequence weighting, position-specific gap penalties and weight matrix choice, Nucleic Acids Research 22: 4673-4680, 1994); CLUSTALV, (see, Larkin M. A., et al., CLUSTALW2, ClustalW and ClustalX version 2, Bioinformatics 23(21): 2947-2948, 2007); Jotun-Hein, Muscle et al., MUSCLE: a multiple sequence alignment method with reduced time and space complexity, BMC Bioinformatics 5: 113, 2004); Mafft, Kalign, ProbCons, and T-Coffee (see Notredame et al., T-Coffee: A novel method for multiple sequence alignments, Journal of Molecular Biology 302: 205-217, 2000). Exemplary programs that implement one or more of the above algorithms include, but are not limited to MegAlign from DNAStar (DNAStar, Inc. 3801 Regent St. Madison, Wis. 53705), MUSCLE, T-Coffee, CLUSTALX, CLUSTALV, JalView, Phylip, and Discovery Studio from Accelrys (Accelrys, Inc., 10188 Telesis Ct, Suite 100, San Diego, Calif 92121). In a non-limiting example, MegAlign is used to implement the CLUSTALW alignment algorithm with the following parameters: Gap Penalty 10, Gap Length Penalty 0.20, Delay Divergent Seqs (30%) DNA Transition Weight 0.50, Protein Weight matrix Gonnet Series, DNA Weight Matrix IUB.

The term “chromosomal integration” means the process whereby an incoming sequence is introduced into the chromosome of a host cell. The homologous regions of the transforming DNA align with homologous regions of the chromosome. Then, the sequence between the homology boxes can be replaced by the incoming sequence in a double crossover (i.e., homologous recombination). In some embodiments of the present invention, homologous sections of an inactivating chromosomal segment of a DNA construct align with the flanking homologous regions of the indigenous chromosomal region of the microbial chromosome. Subsequently, the indigenous chromosomal region is deleted by the DNA construct in a double crossover.

The term “consensus sequence” or “canonical sequence” refers to an archetypical amino acid sequence against which all variants of a particular protein or sequence of interest are compared. Either term also refers to a sequence that sets forth the nucleotides that are most often present in a polynucleotide sequence of interest. For each position of a protein, the consensus sequence gives the amino acid that is most abundant in that position in the sequence alignment.

The term “conservative substitutions” or “conserved substitutions” refers to, for example, a substitution of an amino acid with a conservative variant.

“Conservative variant” refers to residues that are functionally similar to a given residue. Amino acids within the following groups are conservative variants of one another: glycine, alanine, serine, and proline (very small); alanine, isoleucine, leucine, methionine, phenylalanine, valine, proline, and glycine (hydrophobic); alanine, valine, leucine, isoleucine, methionine (aliphatic-like); cysteine, serine, threonine, asparagine, tyrosine, and glutamine (polar); phenylalanine, tryptophan, tyrosine (aromatic); lysine, arginine, and histidine (basic); aspartate and glutamate (acidic); alanine and glycine; asparagine and glutamine; arginine and lysine; isoleucine, leucine, methionine, and valine; and serine and threonine.

The terms “corresponds to” and “corresponding to” used with reference to an amino acid residue or position refer to an amino acid residue or position in a first protein sequence being positionally equivalent to an amino acid residue or position in a second reference protein sequence by virtue of the fact that the residue or position in the first protein sequence aligns to the residue or position in the reference sequence using bioinformatic techniques, for example, using the methods described herein for preparing a sequence alignment. The corresponding residue in the first protein sequence is then assigned the position number in the second reference protein sequence.

The term “deletion,” when used in the context of an amino acid sequence, means a deletion in or a removal of one or more residues from the amino acid sequence of a corresponding protein, resulting in a mutant protein having at least one less amino acid residue as compared to the corresponding protein. The term can also be used in the context of a nucleotide sequence, which means a deletion in or removal of a nucleotide from the polynucleotide sequence of a corresponding polynucleotide.

The term “DNA construct” and “transforming DNA” (wherein “transforming” is used as an adjective) are used interchangeably herein to refer to a DNA used to introduce sequences into a host cell or organism. Typically a DNA construct is generated in vitro by PCR or other suitable technique(s) known to those in the art. In certain embodiments, the DNA construct comprises a sequence of interest (e.g., an incoming sequence). In some embodiments, the sequence is operably linked to additional elements such as control elements (e.g., promoters, etc.). A DNA construct can further comprise a selectable marker. It can also comprise an incoming sequence flanked by homology targeting sequences. In a further embodiment, the DNA construct comprises other non-homologous sequences, added to the ends (e.g., stuffer sequences or flanks). In some embodiments, the ends of the incoming sequence are closed such that the DNA construct forms a closed circle. The transforming sequences may be wild type, mutant or modified. In some embodiments, the DNA construct comprises sequences homologous to the host cell chromosome. In other embodiments, the DNA construct comprises non-homologous sequences. Once the DNA construct is assembled in vitro it may be used to: 1) insert heterologous sequences into a desired target sequence of a host cell; 2) mutagenize a region of the host cell chromosome (i.e., replace an endogenous sequence with a heterologous sequence); 3) delete target genes; and/or (4) introduce a replicating plasmid into the host.

A polynucleotide is said to “encode” an RNA or a polypeptide if, in its native state or when manipulated by methods known to those of skill in the art, it can be transcribed and/or translated to produce the RNA, the polypeptide, or a fragment thereof. The antisense strand of such a polynucleotide is also said to encode the RNA or polypeptide sequences. As is known in the art, a DNA can be transcribed by an RNA polymerase to produce an RNA, and an RNA can be reverse transcribed by reverse transcriptase to produce a DNA. Thus a DNA can encode an RNA, and vice versa.

The term “expressed genes” refers to genes that are transcribed into messenger RNA (mRNA) and then translated into protein, as well as genes that are transcribed into types of RNA, such as transfer RNA (tRNA), ribosomal RNA (rRNA), and regulatory RNA, which are not translated into protein.

The terms “expression cassette” or “expression vector” refer to a polynucleotide construct generated recombinantly or synthetically, with a series of specified elements that permit transcription of a particular polynucleotide in a target cell. A recombinant expression cassette can be incorporated into a plasmid, chromosome, mitochondrial DNA, plasmid DNA, virus, or polynucleotide fragment. Typically, the recombinant expression cassette portion of an expression vector includes, among other sequences, a polynucleotide sequence to be transcribed and a promoter. In particular embodiments, expression vectors have the ability to incorporate and express heterologous polynucleotide fragments in a host cell. Many prokaryotic and eukaryotic expression vectors are commercially available. Selection of appropriate expression vectors is within the knowledge of those of skill in the art. The term “expression cassette” is also used interchangeably herein with “DNA construct,” and their grammatical equivalents.

“Gene” refers to a polynucleotide (e.g., a DNA segment), which encodes a polypeptide, and may include regions preceding and following the coding regions as well as intervening sequences (introns) between individual coding segments (exons).

The term “homologous genes” refers to a pair of genes from different but related species, which correspond to each other and which are identical or similar to each other. The term encompasses genes that are separated by the speciation process during the development of new species) (e.g., orthologous genes), as well as genes that have been separated by genetic duplication (e.g., paralogous genes).

The term “endogenous protein” refers to a protein that is native or naturally occurring. “Endogenous polynucleotide” refers to a polynucleotide that is in the cell and was not introduced into the cell using recombinant engineering techniques; for example, a gene that was present in the cell when the cell was originally isolated from nature.

The term “heterologous” used with reference to a protein or a polynucleotide in a host cell refers to a protein or a polynucleotide that does not naturally occur in the host cell.

The term “heterologous” used to describe two different amino acid or nucleic acid sequences refers to two sequences that are not naturally present together in the same protein or nucleic acid. The term “heterologous” used to describe two different protein domains refers to two protein domains that are not naturally present together in the same protein. As used herein, “domain” refers to any portion of protein that confers a particular structural and/or functional characteristic to a protein. Exemplary protein domains include signal peptides, extracellular domains, transmembrane domains, cytoplasmic domains, catalytic domains, affinity tags, and linkers, among others.

The term “homologous recombination” refers to the exchange of DNA fragments between two DNA molecules or paired chromosomes at sites of identical or nearly identical nucleotide sequences. In certain embodiments, chromosomal integration is homologous recombination.

The term “homologous sequences” as used herein refers to a polynucleotide or polypeptide sequence having, for example, about 100%, about 99% or more, about 98% or more, about 97% or more, about 96% or more, about 95% or more, about 94% or more, about 93% or more, about 92% or more, about 91% or more, about 90% or more, about 88% or more, about 85% or more, about 80% or more, about 75% or more, about 70% or more, about 65% or more, about 60% or more, about 55% or more, about 50% or more, about 45% or more, or about 40% or more sequence identity to another polynucleotide or polypeptide sequence when optimally aligned for comparison. In particular embodiments, homologous sequences can retain the same type and/or level of a particular activity of interest. In some embodiments, homologous sequences have between 85% and 100% sequence identity, whereas in other embodiments there is between 90% and 100% sequence identity. In particular embodiments, there is 95% and 100% sequence identity.

“Homology” refers to sequence similarity or sequence identity. Homology is determined using standard techniques known in the art (see, e.g., Smith and Waterman, Adv. Appl. Math., 2:482, 1981; Needleman and Wunsch, J. Mol. Biol., 48:443, 1970; Pearson and Lipman, Proc. Natl. Acad. Sci. USA 85:2444, 1988; programs such as GAP, BESTFIT, FASTA, and TFASTA in the Wisconsin Genetics Software Package (Genetics Computer Group, Madison, Wis.); and Devereux et al., Nucl. Acid Res., 12:387-395, 1984). A non-limiting example includes the use of the BLAST program (Altschul et al., Gapped BLAST and PSI-BLAST: a new generation of protein database search programs, Nucleic Acids Res. 25:3389-3402, 1997) to identify sequences that can be said to be “homologous.” A recent version such as version 2.2.16, 2.2.17, 2.2.18, 2.2.19, or the latest version, including sub-programs such as blastp for protein-protein comparisons, blastn for nucleotide-nucleotide comparisons, tblastn for protein-nucleotide comparisons, or blastx for nucleotide-protein comparisons, and with parameters as follows: Maximum number of sequences returned 10,000 or 100,000; E-value (expectation value) of 1e-2 or 1e-5, word size 3, scoring matrix BLOSUM62, gap cost existence 11, gap cost extension 1, may be suitable. An E-value of 1e-5, for example, indicates that the chance of a homologous match occurring at random is about 1 in 10,000, thereby marking a high confidence of true homology.

The term “host strain” or “host cell” refers to a suitable host for an expression vector comprising a DNA of the present invention. The host may comprise any organism, without limitation, capable of containing and expressing the nucleic acids or genes disclosed herein. The host may be prokaryotic or eukaryotic, single-celled or multicellular, including mammalian cells, plant cells, fungi, etc. Examples of single-celled hosts include cells of Escherichia, Salmonella, Bacillus, Clostridium, Streptomyces, Staphyloccus, Neisseria, Lactobacillus, Shigella, and Mycoplasma. Suitable E. coli strains (among a great many others) include BL21(DE3), C600, DH5αF′, HB101, JM83, JM101, JM103, JM105, JM107, JM109, JM110, MC1061, MC4100, MM294, NM522, NM554, TGI, χ1776, XL1-Blue, and Y1089+, all of which are commercially available.

The term “identical” (or “identity”), in the context of two polynucleotide or polypeptide sequences, means that the residues in the two sequences are the same when aligned for maximum correspondence, as measured using a sequence comparison or analysis algorithm such as those described herein. For example, if when properly aligned, the corresponding segments of two sequences have identical residues at 5 positions out of 10, it is said that the two sequences have a 50% identity. Most bioinformatic programs report percent identity over aligned sequence regions, which are typically not the entire molecules. If an alignment is long enough and contains enough identical residues, an expectation value can be calculated, which indicates that the level of identity in the alignment is unlikely to occur by random chance.

The term “insertion,” when used in the context of an amino acid sequence, refers to an insertion of an amino acid with respect to the amino acid sequence of a corresponding polypeptide, resulting in a mutant polypeptide having an amino acid that is inserted between two existing contiguous amino acids, i.e., adjacent amino acids residues, which are present in the corresponding polypeptide. The term “insertion,” when used in the context of a polynucleotide sequence, refers to an insertion of one or more nucleotides in the corresponding polynucleotide between two existing contiguous nucleotides, i.e., adjacent nucleotides, which are present in the corresponding polynucleotides.

The term “introduced” refers to, in the context of introducing a polynucleotide sequence into a cell, any method suitable for transferring the polynucleotide sequence into the cell. Such methods for introduction include but are not limited to protoplast fusion, transfection, transformation, conjugation, and transduction (see, e.g., Ferrari et al., Genetics, in Hardwood et al, (eds.), Bacillus, Plenum Publishing Corp., pp. 57-72, 1989).

The term “isolated” or “purified” means a material that is removed from its original environment, for example, the natural environment if it is naturally occurring, or a cultivation broth if it is produced in a recombinant host cell cultivation medium. A material is said to be “purified” when it is present in a particular composition in a higher concentration than the concentration that exists prior to the purification step(s). For example, with respect to a composition normally found in a naturally occurring or wild type organism, such a composition is “purified” when the final composition does not include some material from the original matrix. As another example, where a composition is found in combination with other components in a recombinant host cell cultivation medium, that composition is purified when the cultivation medium is treated in a way to remove some component of the cultivation, for example, cell debris or other cultivation products, through, for example, centrifugation or distillation. As another example, a naturally occurring polynucleotide or polypeptide present in a living animal is not isolated, but the same polynucleotide or polypeptide, separated from some or all of the coexisting materials in the natural system, is isolated, whether such process is through genetic engineering or mechanical separation. Such polynucleotides can be parts of vectors. Alternatively, such polynucleotides or polypeptides can be parts of compositions. Such polynucleotides or polypeptides can be considered “isolated” because the vectors or compositions comprising thereof are not part of their natural environments. In another example, a polynucleotide or protein is said to be purified if it gives rise to essentially one band in an electrophoretic gel or a blot.

The term “mutation” refers to, in the context of a polynucleotide, a modification to the polynucleotide sequence resulting in a change in the sequence of a polynucleotide with reference to a corresponding polynucleotide sequence. A mutation to a polynucleotide sequence can be an alteration that does not change the encoded amino acid sequence, for example, with regard to codon optimization for expression purposes, or that modifies a codon in such a way as to result in a modification of the encoded amino acid sequence. Mutations can be introduced into a polynucleotide through any number of methods known to those of ordinary skill in the art, including random mutagenesis, site-specific mutagenesis, oligonucleotide directed mutagenesis, gene shuffling, directed evolution techniques, combinatorial mutagenesis, site saturation mutagenesis among others.

“Mutation” or “mutated” means, in the context of a protein, a modification to the amino acid sequence resulting in a change in the sequence of a protein with reference to a corresponding protein sequence. A mutation can refer to a substitution of one amino acid with another amino acid, an insertion of one or more amino acid residues, or a deletion of one or more amino acid residues. A mutation can include the replacement of an amino acid with a non-natural amino acid, or with a chemically-modified amino acid or like residues. A mutation can also be a truncation (e.g., a deletion or interruption) in a sequence or a subsequence from the corresponding sequence. A mutation can be made by modifying the DNA sequence corresponding to the corresponding protein. Mutations can be introduced into a protein sequence by known methods in the art, for example, by creating synthetic DNA sequences that encode the mutation with reference to corresponding proteins, or chemically altering the protein itself. A “mutant” as used herein is a protein comprising a mutation.

A “naturally occurring equivalent,” in the context of the present invention, refers to a naturally occurring ACE2 protein, or a portion thereof that comprises a naturally occurring residue.

The term “operably linked,” in the context of a polynucleotide sequence, refers to the placement of one polynucleotide sequence into a functional relationship with another polynucleotide sequence. For example, a DNA encoding a secretory leader (e.g., a signal peptide) is operably linked to a DNA encoding a polypeptide if it is expressed as a preprotein that participates in the secretion of the polypeptide. A promoter or an enhancer is operably linked to a coding sequence if it affects the transcription of the sequence. A ribosome binding site is operably linked to a coding sequence if it is positioned so as to facilitate translation. Generally, “operably linked” means that the DNA sequences being linked are contiguous, and, in the case of a secretory leader, contiguous and in the same reading frame.

The term “optimal alignment” refers to the alignment giving the highest overall alignment score.

“Overexpressed” or “overexpression” in a host cell occurs if the enzyme is expressed in the cell at a higher level than the level at which it is expressed in a corresponding wild-type cell.

The terms “percent sequence identity,” “percent amino acid sequence identity,” “percent gene sequence identity,” and/or “percent polynucleotide sequence identity,” with respect to two polypeptides, polynucleotides and/or gene sequences (as appropriate), refer to the percentage of residues that are identical in the two sequences when the sequences are optimally aligned. Thus, 80% amino acid sequence identity means that 80% of the amino acids in two optimally aligned polypeptide sequences are identical. The percent identities expressed herein with respect to a given named reference sequence are determined over the entire reference sequence, rather than only a portion thereof. Thus, an amino acid sequence at least about 80% identical to positions 18-615 of SEQ ID NO:1, for example, is at least about 80% identical to the entire sequence of positions 18-615 of SEQ ID NO:1, as opposed merely to subsequences thereof.

The term “plasmid” refers to a circular double-stranded (ds) DNA construct used as a cloning vector, and which forms an extrachromosomal self-replicating genetic element in some eukaryotes or prokaryotes, or integrates into the host chromosome.

A “production host” is a cell used to produce products. As disclosed herein, a production host is modified to express or overexpress selected genes, and/or to have attenuated expression of selected genes. Non-limiting examples of production hosts include plant, animal, human, bacteria, yeast, cyanobacteria, algae, and/or filamentous fungi cells.

A “promoter” is a polynucleotide sequence that functions to direct transcription of a downstream gene. In preferred embodiments, the promoter is appropriate to the host cell in which the target gene is being expressed. The promoter, together with other transcriptional and translational regulatory polynucleotide sequences (also termed “control sequences”) is necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

The terms “protein” and “polypeptide” are used interchangeably herein. The 3-letter code as well as the 1-letter code for amino acid residues as defined in conformity with the IUPAC-IUB Joint Commission on Biochemical Nomenclature (JCBN) is used throughout this disclosure. It is also understood that a polypeptide may be coded for by more than one polynucleotide sequence due to the degeneracy of the genetic code. An enzyme is a protein. The terms “amino acid sequence” and “polypeptide sequence” are used interchangeably herein.

The terms “nucleic acid” and “polynucleotide” are used interchangeably herein.

The term “recombinant,” when used to modify the term “cell” or “vector” herein, refers to a cell or a vector that has been modified by the introduction of a heterologous polynucleotide sequence, or that the cell is derived from a cell so modified. Thus, for example, recombinant cells express genes that are not found in identical form within the native (non-recombinant) form of the cells or express, as a result of deliberate human intervention, native genes that are otherwise abnormally expressed, underexpressed or not expressed at all. The terms “recombinant,” used with respect to proteins and nucleic acids refers to mutant proteins and nucleic acids, respectively.

The terms “regulatory segment,” “regulatory sequence,” or “expression control sequence” refer to a polynucleotide sequence that is operatively linked with another polynucleotide sequence that encodes the amino acid sequence of a polypeptide chain to effect the expression of that encoded amino acid sequence. The regulatory sequence can inhibit, repress, promote, or even drive the expression of the operably-linked polynucleotide sequence encoding the amino acid sequence.

The term “substantially identical,” in the context of two polynucleotides or two polypeptides refers to a polynucleotide or polypeptide that comprises at least 70% sequence identity, for example, at least 75%, at least 80%, at least 85%, at least 90%, at least 91%, at least 92%, at least 93%, at least 94%, at least 95%, at least 96%, at least 97%, at least 98%, or at least 99% sequence identity as compared to a reference sequence using the programs or algorithms (e.g., BLAST, ALIGN, CLUSTAL) using standard parameters.

“Substantially purified” means molecules that are at least about 60% free, preferably at least about 75% free, about 80% free, about 85% free, and more preferably at least about 90% free from other components with which they are naturally associated. As used herein, the term “purified” or “to purify” also refers to the removal of contaminants from a sample.

“Substitution” means replacing an amino acid in the sequence of a corresponding protein with another amino acid at a particular position, resulting in a mutant of the corresponding protein. The amino acid used as a substitute can be a naturally occurring amino acid, or can be a synthetic or non-naturally occurring amino acid.

The term “transformed” or “stably transformed” cell refers to a cell that has a non-native (heterologous) polynucleotide sequence integrated into its genome or as an episomal plasmid that is maintained for at least two generations.

“Vector” refers to a polynucleotide construct designed to introduce polynucleotides into one or more cell types. Vectors include cloning vectors, expression vectors, shuttle vectors, plasmids, cassettes and the like. In some embodiments, the polynucleotide construct comprises a polynucleotide sequence encoding a mutant ACE2 protein that is operably linked to a suitable prosequence capable of effecting the expression of the polynucleotide or gene in a suitable host.

“Wild type” means, in the context of gene or protein, a polynucleotide or protein sequence that occurs in nature. In some embodiments, the wild-type sequence refers to a sequence of interest that is a starting point for protein engineering. “Wild type” is used interchangeably with “native.”

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Summary

Mutants of the angiotensin converting-enzyme 2 (ACE2) carboxypeptidase possessing enhanced hydrolytic activity and specificity hold potential to beneficially modulate the angiotensin receptor (ATR)/Mas receptor (MasR) therapeutic axis with increased efficacy and reduced potential side effects relative to wild type ACE2. In pursuing this goal, we established a yeast surface display-based liquid chromatography (LC) screen that identified ACE2 mutants with improved target peptide substrate, angiotensin II (Ang-II), activity and specificity relative to Apelin-13, an off-target peptide substrate.

Screening of yeast-displayed ACE2 active site residue saturation mutant libraries revealed at least six substitution-tolerant positions that can be mutated to enhance ACE2's activity profile. Double mutant libraries combining substitutions at three positions, M360, T371 and Y510, yielded candidate improved ACE2 mutants that were purified from mammalian cell culture supernatant at approximately 10 mg/L yield and >90% homogeneity.

Relative to wild type, the leading mutant, T371L/Y510I, has seven-fold increased k_{cat} toward Ang-II and six-fold decreased k_{cat}/Kₘ for Apelin-13 hydrolysis. In single substrate hydrolysis assays performed at physiologically relevant substrate concentrations T371L/Y510I hydrolyzes as much or more Ang-II than wild type with concomitant Ang-II.Apelin-13 specificity improvements reaching 30-fold. Additionally, T371L/Y510I hydrolyzed Ang-II at rates equal to or greater than wild type, with reductions in Apelin-13 hydrolysis of up to 80 percent, in multiplex assays featuring a mixture of peptides relevant to the ATR/MasR therapeutic axis. Collectively, the above efforts have both delivered ATR/MasR axis-acting therapeutic candidates and established the feasibility of developing ACE2 variants for use in other biomedical applications such as localized activation of peptide-based prodrugs.

The angiotensin converting enzyme 2 (ACE2) carboxypeptidase is being clinically trialed for the treatment of acute respiratory distress syndrome related to traumatic injuries and viral infections. We have developed a yeast surface display-based screening methodology that enables isolation of mutant ACE2s that possess enhanced peptide substrate hydrolysis activity and specificity profiles with respect to substrates relevant to respiratory distress therapy. These efforts have both yielded mutant ACE2s with near term potential as better than wild type therapeutic agents and established the utility of our screening methodology in developing additional ACE2 variants with modified properties that make them useful for novel ACE2 biomedical applications including localized cerebral vasodilation in treating Alzheimer's Disease and targeted activation of peptide-based prodrugs for cancer therapy.

### Introduction

Use of gene mutagenesis and mutant property screening to modify peptidase activity and specificity profiles can yield novel enzymes that create new vistas on disease treatment and prevention. In these examples we utilize a yeast surface display system [1,2] to develop angiotensin-converting enzyme 2 (ACE2) mutants that possess increased activity and specificity toward the angiotensin-II peptide (Ang-II), an ACE2 substrate whose hydrolysis is associated with salutary blood flow increase [3], suppression of inflammation [4] and attenuation of pathological tissue remodeling after acute injury [3], relative to Apelin-13, an ACE2 substrate possessing a number of analeptic in vivo roles that may be compromised upon hydrolysis [5]. This outcome provides new drug candidates for treating respiratory viral infections [6], acute respiratory distress syndrome [7], diabetes [8] and Alzheimer's Disease [4]. Additionally, the enhanced activity profiles of these variants establishes the feasibility of using our ACE2 mutant library screening methodology to develop peptidases that hydrolyze non-native ACE2 substrates; such enzymes could be used to achieve localized in vivo activation of peptide-based prodrugs [9].

ACE2 is a zinc-dependent mono-carboxypeptidase found in both membrane bound and freely circulating forms in vivo [10]. The full-length membrane bound protein features, from N- to C-terminus: an ˜ 600 amino acid peptidase domain, an ˜ 120 AA collectrin-like domain that mediates formation of enzyme dimerization, a twenty amino acid transmembrane domain and a forty amino cytosolic domain [1,11,12]. Recombinant forms of ACE2 employed in or under development for therapeutic applications are comprised of either both the peptidase and collectrin domains [6,7] or the peptidase domain only [11]; our efforts are focused on developing ACE2 candidate therapeutics comprised of only the peptidase domain.

At least eight peptides found in circulation and/or tissues in vivo have been identified as ACE2 substrates [5,13]. The biological effects of ACE2-mediated hydrolysis of Ang-II, an octapeptide with sequence DRVYIHPF (residues 1-8 of SEQ ID NO:3) that is produced via angiotensin-converting enzyme 1 (ACE) hydrolysis of the Ang-I decapeptide that features sequence DRVYIHPFHL (SEQ ID NO:3), are well-characterized. ACE2-mediated removal of Ang-II's C-terminal Phe residue yields Ang1-7; this septapeptide [4,14,15] acts through the G protein-coupled receptor Mas (MasR) to exert vasodilative, anti-inflammatory, and anti-fibrotic effects. Ang-II acts on both the type1 and type2 angiotensin receptors (AT1R and AT2R). Vasoconstriction, inflammation, tissue fibrosis and compromise of vascular endothelial cell function are among the effects arising from [4,14,15,16] Ang-II activation of ATR receptors. Given the above observations, augmenting native ACE2 activity to simultaneously reduce levels of Ang-II and increase Ang1-7 concentration is being pursued as a strategy for treating numerous health conditions.

Regardless of ACE2's possessing activity toward multiple peptide substrates without known roles in the Ang-II/ATR/MasR biological axis, to our knowledge there have not been efforts to enhance ACE2's therapeutic efficacy by modulating its activity and specificity profile for increased activity toward Ang-II and decreased activity toward other natural substrates such as Apelin-13. ACE2 mutagenesis studies conducted to date have been fundamental in nature and focused on identifying ACE2 residues with respective roles in the enzyme's Zn²⁺ and Cl⁻ binding properties [17,18].

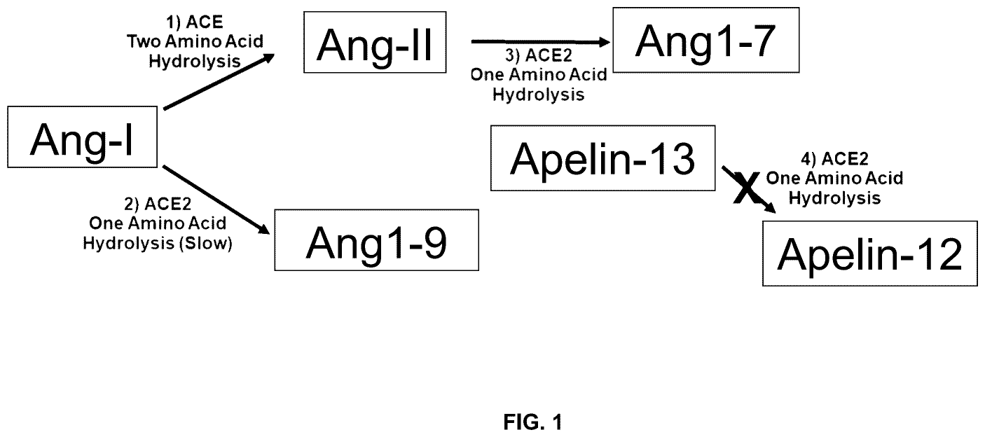

Apelins, which exist in the aforementioned thirteen residue and longer isoforms, all of which share a C-terminus recognized by ACE2, are the ACE2 peptide substrates for which potential avenues by which ACE2-mediated hydrolysis could lead to deleterious side effects, in particular myocardial dysfunction, have been most well-defined [5,19,20]. Additionally, ACE2's catalytic efficiency, i.e., k_{cat}/Kₘ, toward Ang-II is similar to its efficiency toward Apelins [13,19] and the concentration of Apelins, which are present at single-digit nM levels in human plasma [21], are higher than both the 10-50 μM Ang-II concentrations observed in blood [22] and the 0.2-0.3 nM levels of Ang-II found in human tissues [23,24]; these observations support the hypothesis that Apelins can be potent competitors of ACE-2 activity toward Ang-II under the mixed peptide substrate conditions that exist in vivo. Taken together, the above statements motivate identification of ACE2 mutants with greater than wild type Ang-II.Apelin hydrolytic activity ratios, and ideally equal to or greater than wild type Ang-II hydrolytic activity, as both an ACE2-based therapeutic development objective and the context for establishing the functionality and utility of a first-ever screening platform for modifying ACE2's activity and specificity profile. The schematic of FIG. 1 interleaves the preceding remarks in enlightening the collection of mechanisms by which ACE2 variants can provide increased therapeutic efficacy and improved safety profiles relative to the wild type enzyme.

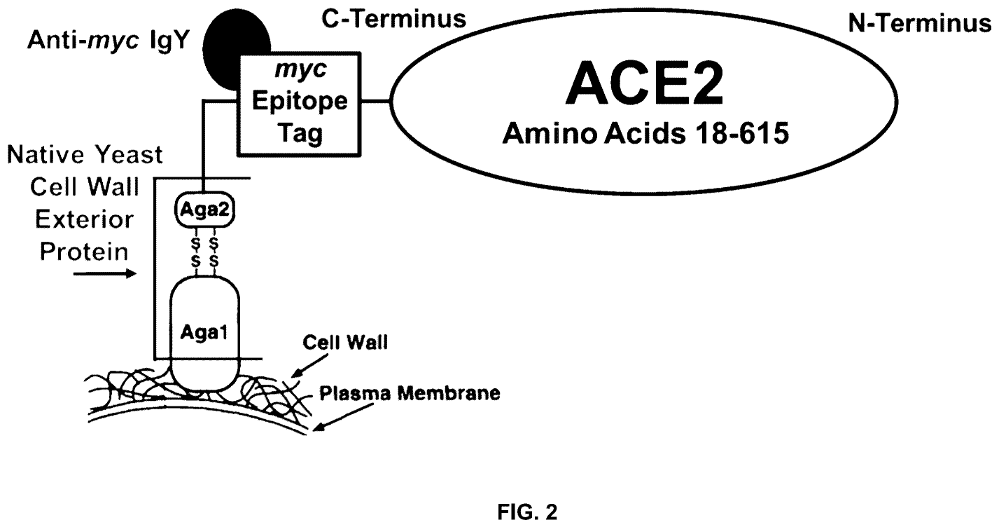

Although internally quenched peptide substrates that fluoresce upon hydrolysis by ACE2 have been utilized in studies focused on measuring relative activities of ACE2 isoforms, e.g., peptidase domain only versus entire extracellular domain [11] or optimizing recombinant ACE2 expression yields [25], the sequences of these peptides are unrelated to Ang-II and other natural ACE2 substrates. As such, although these fluorescent substrates could be used in conjunction with fluorescence activated cell sorting (FACS)- or microfluidic droplet-based approaches [26,27] to screen for ACE2 mutants with modified catalytic properties it is ambiguous as to how effective such screens would be in identifying ACE2 mutants with improved Ang-II hydrolysis and specificity profiles. Yeast surface display [1,2], which allows one to express active enzymes on the yeast surface (FIG. 2) and subsequently transfer these enzymes into contaminant-free ACE2 hydrolysis reaction buffer by simple centrifugation and resuspension, combined with high performance liquid chromatography (LC) analysis of ACE2-mediated hydrolysis of authentic peptide substrates, e.g., Ang-II and the commercially available and inexpensive Apelin-13, offers a viable alternative to the aforementioned procedures for isolating ACE2 variants with enhanced activity and specificity characteristics.

The combined yeast display-LC peptidase screening approach has a capacity of approximately fifty mutants per day and thus is not compatible with screening of random mutant libraries, an enzyme engineering strategy commonly applied when microfluidics or FACS procedures that accommodate screening rates of thousands of droplets or cells per second are available. LC screening throughput is however, not an insurmountable challenge to creating ACE2 variants with enhanced activity and specificity because examination of an ACE2-small molecule inhibitor co-crystal structure and sequence alignment against structurally similar peptidases allows one to identify ACE2 amino acid residues that are likely to both play roles in substrate hydrolysis and be tolerant to substitutions [28]. Construction of ACE2 site-directed mutant, e.g., NNB codon [29], yeast display libraries at such residue positions and subsequent LC screening allows one to, without unduly large time and labor inputs, identify ACE2 substitutions that enhance peptidase activity and specificity profiles.

In these examples, we develop a yeast display-LC screening methodology and use it to screen site-directed ACE2 mutant libraries; screening led to the identification of ACE2 mutants possessing both improved specificity toward Ang-II relative to Apelin-13 and Ang-II hydrolytic activity greater than or equal to that of wild type ACE2. These enhanced properties were observed for the ACE2 mutants regardless of whether evaluated in yeast-displayed or soluble, purified formats. The outcomes presented here can have an important impact in achieving broad deployment of ACE2-based approaches for prevention and treatment of a wide range of health conditions.

### Results

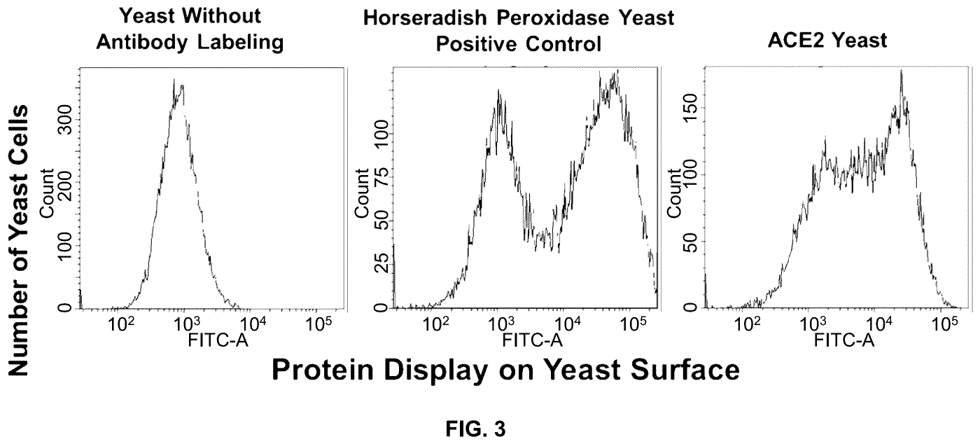

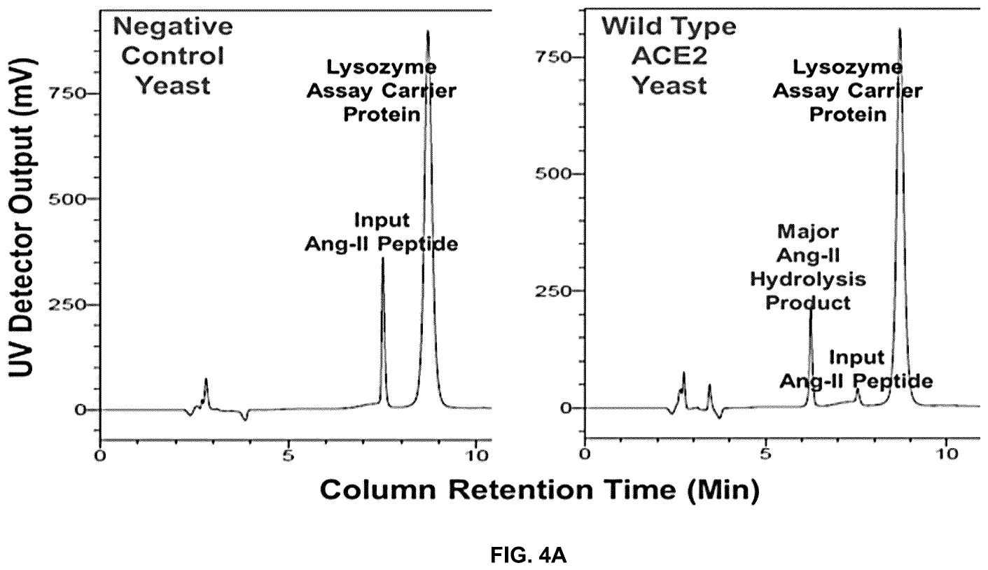

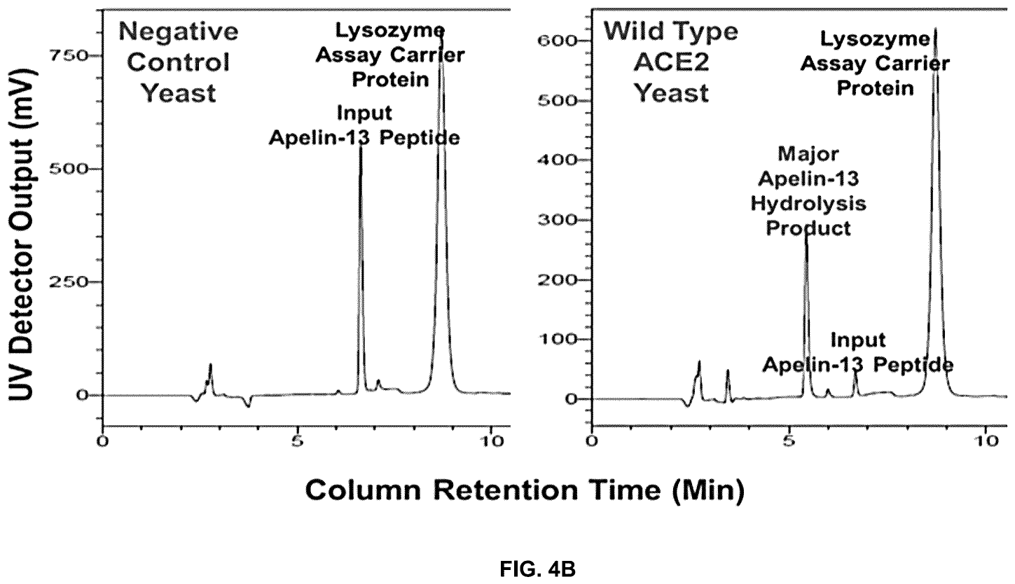

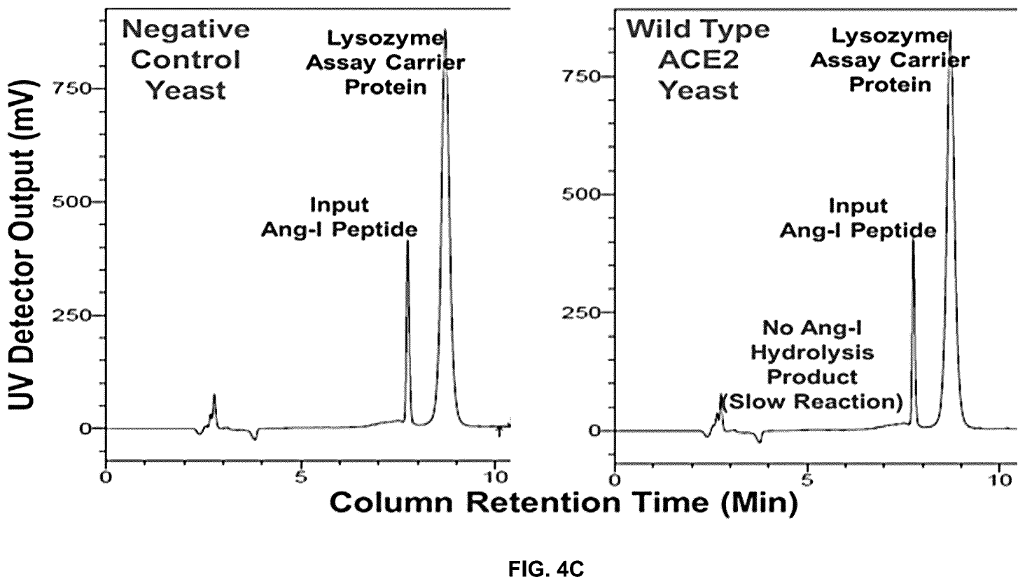

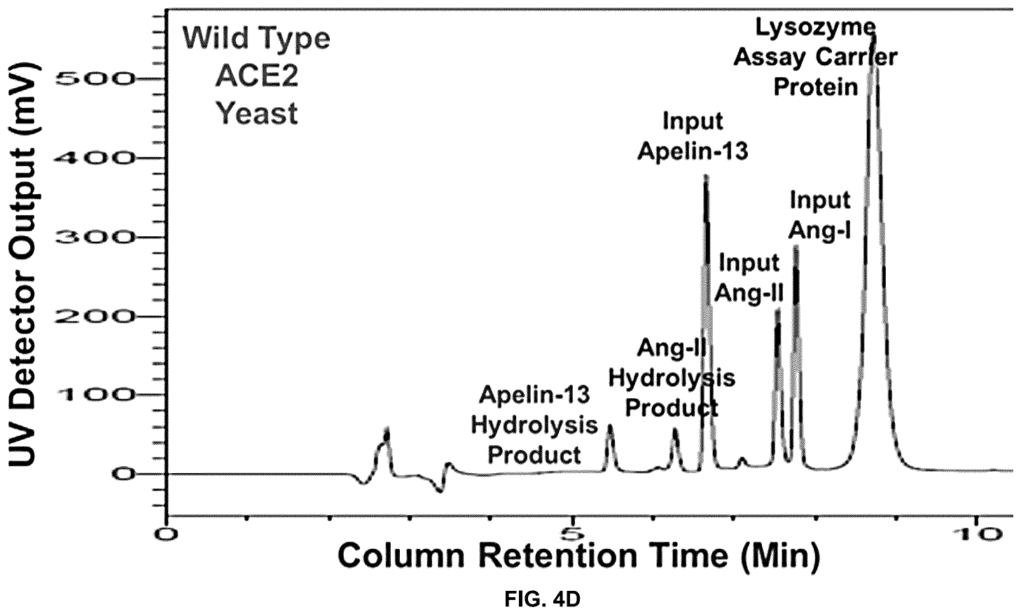

A Saccharomyces cerevisiae codon-optimized gene encoding the human ACE2 (SEQ ID NO:1) peptidase domain, amino acids 18-615, was well-expressed as a C-terminal fusion to the yeast cell surface (FIG. 3). LC analysis of reaction supernatants for ACE2-displaying yeast incubated with respective Ang-II and Apelin-13 peptides showed that yeast suspended in ACE2 reaction buffer at a density of approximately 10⁶ cells per mL hydrolyze more than 95% of these peptide substrates during a three-hour room temperature reaction (FIGS. 4A-4D). No substrate hydrolysis was observed for incubations with negative control yeast. Neither ACE2-displaying yeast nor negative control yeast hydrolyzed the Ang-I peptide; this outcome was expected given that the ACE2 hydrolysis k_{cat}/Kₘ value for Ang-I is reported as being nearly 1,000-fold lower than those for Apelin-13 and Ang-II [13]. Given that Ang-I, Ang-II and Apelin-13 are all present in both blood and tissues in vivo we were motivated to develop a multiplex substrate hydrolysis assay for use in ACE2 site-directed mutant library screening. Surprisingly, Ang-I appeared to act as an inhibitor of ACE2-mediated Ang-II and Apelin-13 hydrolysis under these multiplex reaction conditions as respective conversions of Ang-II and Apelin-13 were reduced approximately five-fold relative to the above single substrate hydrolysis reactions (FIGS. 4A-4D).

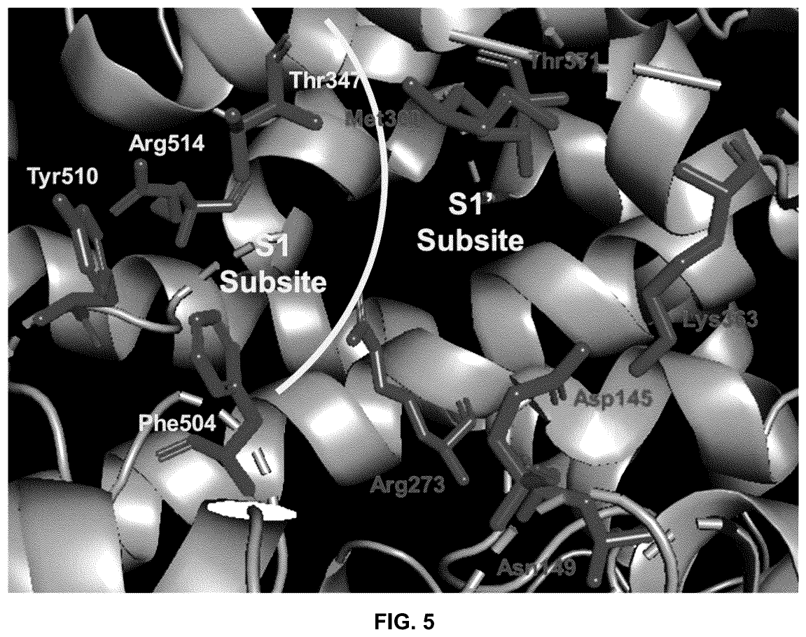

Having identified multiplex substrate reaction conditions that provided a substantial margin for both increases and decreases in Ang-II and Apelin-13 hydrolysis relative to wild type ACE2, we chose ACE2 residue positions at which to introduce NNB (N denotes all four nucleotides while B denotes C, G or T) codons in the context of making single site-directed yeast display ACE2 mutant libraries. Guided by the crystal structure of ACE2 and observation of ACE2 active site residues conserved across structurally similar carboxypeptidases [30], where positions at which variation across homologues was observed were given preference over conserved positions, we chose to introduce NNB codons at ACE2 residues E145, N149, R273, T347, M360, K363, T371, F504, Y510 and R514 (FIG. 5).

Between five and fifteen clones for each site-directed ACE2 library were screened in substrate multiplex assays that contained 25 μM Ang-I, 25 μM Ang-II and 25 μM Apelin-13. The broad LC signal dynamic range (FIGS. 4A-4D) observed for hydrolysis assays using these substrate concentrations motivated their employment regardless of the concentrations being higher than the published [13,19] in vitro Kₘ values (2-20 μM) and the picomolar-nanomolar peptide substrate concentration ranges [21-24] observed in blood and tissues. Absolute hydrolysis of Ang-II and Ang-II:Apelin-13 hydrolysis ratio were the metrics for determining whether screening of additional clones for a particular site-directed single mutant library was warranted.

We found that more than half of the sampled clones from the R273, T347, F504 and R514 site-directed mutant libraries had hydrolytic activity toward Ang-II that was less than fifty percent of the wild type ACE2 activity and chose not to screen additional clones from these libraries (Table 1). The majority of assayed clones from the E145, N149 and K363 libraries possessed substrate hydrolysis profiles very similar to wild type ACE2 and thus further characterization of clones from these libraries was not performed. Initial sampling of clones from the M360, T371 and Y510 libraries revealed both a high fraction of catalytically active variants and variants with substrate hydrolysis metrics that were improved relative to wild type (Table 1).

TABLE 1
Peptide hydrolysis profiles for clones characterized during initial
LC screening of ACE2 site-directed single mutant libraries. Functionally
impaired clones defined as ACE2 clones with less than 50% wild type
ACE2 activity toward both Ang-II and Apelin-13 peptides. Improved
clones defined as ACE2 variants with greater than 50% wild type
ACE2 activity toward Ang-II and Ang-II:Apelin-13 hydrolysis ratio
increased two-fold relative to the ratio for wild type ACE2.

| ACE2 Position | % Functionally | % Clones Similar | % Improved |
|---|---|---|---|
| Mutated | Impaired Clones | to Wild Type | Clones |
| E145 | 0/5 (0%) | 5/5 (100%) | 0/5 (0%) |
| N149 | 0/5 (0%) | 5/5 (100%) | 0/5 (0%) |
| R273 | 4/5 (80%) | 1/5 (20%) | 0/5 (0%) |
| T347 | 3/5 (60%) | 2/5 (40%) | 0/5 (0%) |
| M360 | 2/12 (17%) | 6/12 (50%) | 4/12 (33%) |
| K363 | 0/5 (0%) | 5/5 (100%) | 0/5 (0%) |
| T371 | 0/2 (0%) | 10/12 (83%) | 2/12 (17%) |
| F504 | 5/5 (100%) | 0/5 (0%) | 0/5 (0%) |
| Y510 | 7/15 (47%) | 0/15 (0%) | 8/15 (53%) |
| R514 | 7/10 (70%) | 3/10 (30%) | 0/10 (0%) |

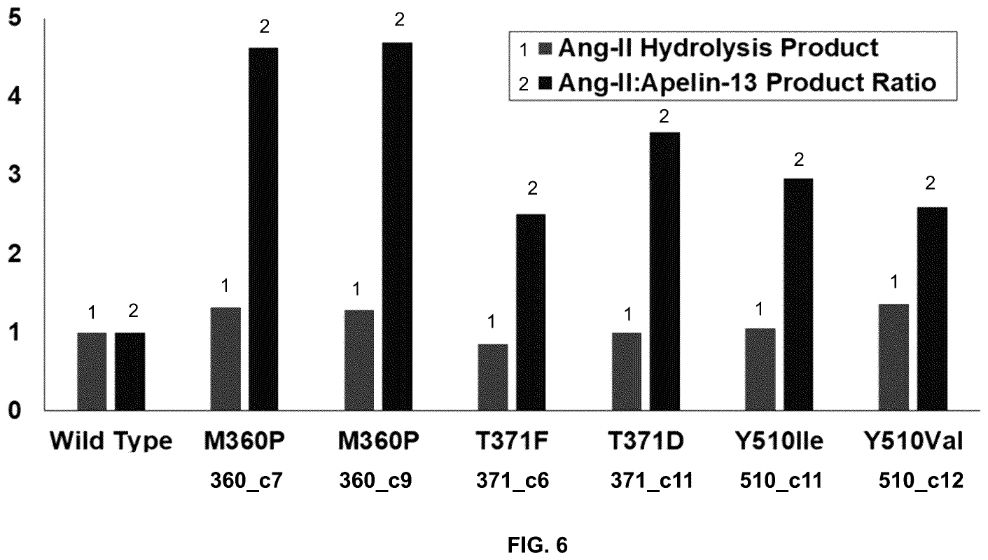

Screening of twelve, twelve and fifteen clones from the M360, T371 and Y510 mutant libraries yielded two variants for each library that possessed markedly improved substrate hydrolysis profiles relative to the other clones sampled from these respective libraries (FIG. 6). One of the two leading clones, i.e., position 360 library clone 7, position 371 library clone 11 and position 510 library clone 11 for each library was chosen as the parent clone for construction of ACE2 double mutant libraries. Six double mutant libraries were generated: 360-clone 7 with NNB codon at position 371, 360-clone7 with NNB at position 510, 371-clone 11 with NNB at position 360, 371-clone 11 with NNB at 510, 510-clone 11 with NNB at 360 and 510-clone 11 with NNB at 371. Single mutant parent clones were not sequenced prior to double mutant library construction. Subsequent sequencing showed that 360-clone 7 (360_c7) in FIG. 6 carried an M360P mutation, 360-clone 9 (360_c9) in FIG. 6 carried an M360P mutation, 371-clone 6 (371_c6) in FIG. 6 carried a T371D mutation, 371-clone 11 (371_c11) in FIG. 6 carried a T371F mutation, 510-clone 11 (510_c11) in FIG. 6 carried a Y510I mutation, and 510-clone 12 (510_c12) in FIG. 6 carried a Y510V mutation.

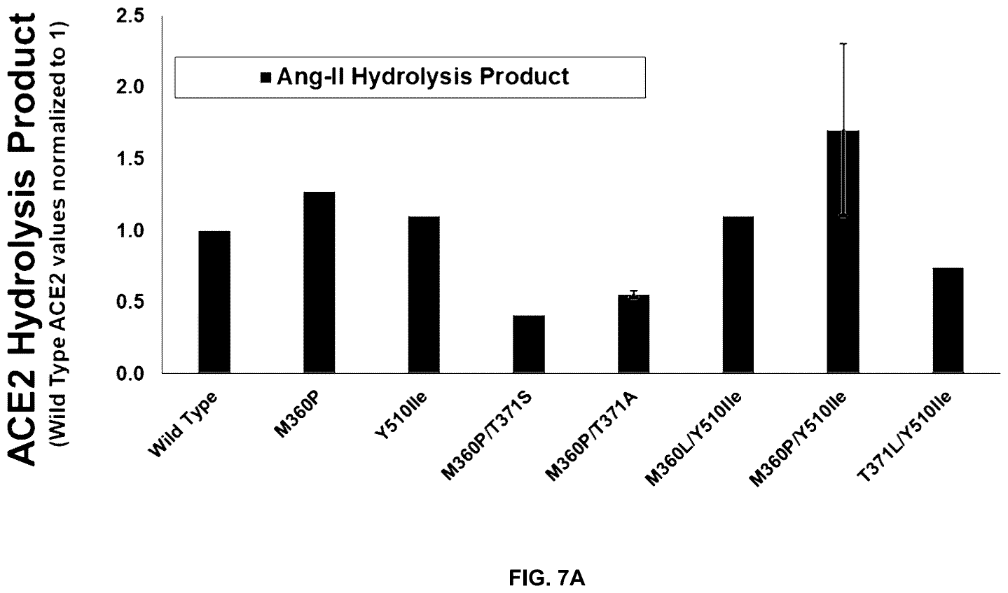

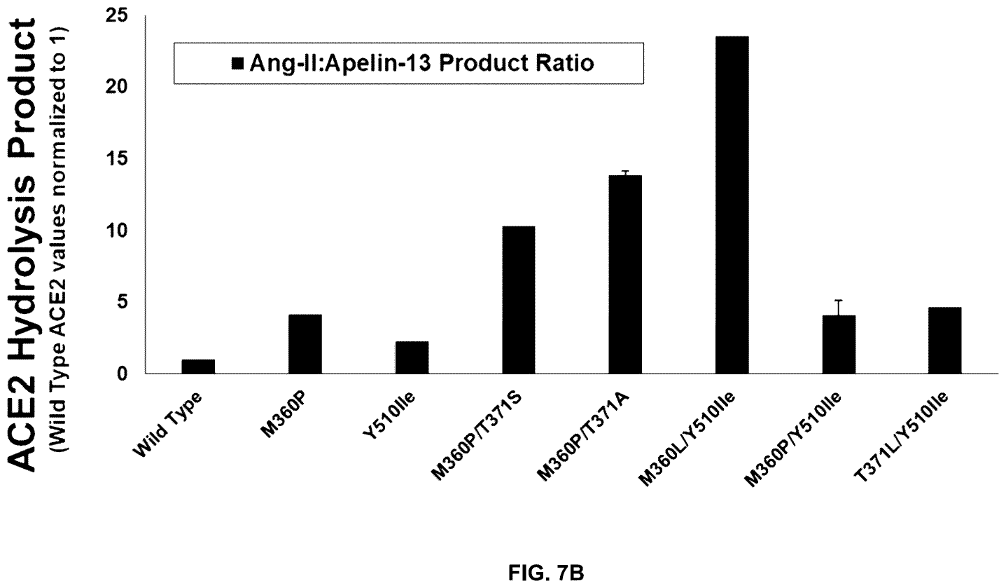

Five clones for each double mutant library were evaluated using the multiplex substrate hydrolysis assay and LC analysis as above to obtain estimates of the fractions of functionally impaired and improved clones in each library; these estimates were used in defining the strategy for screening of additional clones. As shown in Table 2, the 360-clone 7/NNB 371, 510-clone 11/NNB360 and 510-clone 11/NNB 371 double mutant libraries warranted further screening by virtue of containing a high fraction of active clones. Screening of between eighteen and twenty-five additional clones from each of these libraries and subsequent sequencing of the most promising clones yielded the collection of five leading double mutants: M360P/T371A, M360P/T371S, M360P/Y510I, M360L/Y510I and T371L/Y510I with activity profiles, as observed in multiplex substrate hydrolysis assays, shown in FIGS. 7A and 7B.

TABLE 2
Peptide hydrolysis profiles for clones characterized during initial
LC screening of ACE2 site-directed double mutant libraries.
Functionally impaired clones defined as ACE2 clones with less
than 50% wild type ACE2 activity toward both Ang-II and Apelin-13
peptides. Improved clones defined as ACE2 double mutants with
greater than 50% parental single mutant activity toward Ang-II
and Ang-II:Apelin-13 hydrolysis ratio increased two-fold relative
to the ratio for the parental single mutant.

| | | % Clones | |
|---|---|---|---|
| ACE2 Double | % Functionally | Similar | |
| Mutant | Impaired | to Single | % Improved |
| Library | Clones | Mutant Parent | Clones |
| 360-clone 7/NNB 371 | 2/5 (40%) | 3/5 (60%) | 0/5 (0%) |
| 360-clone 7/NNB 510 | 4/5 (80%) | 1/5 (20%) | 0/5 (0%) |
| 371-clone 11/NNB 360 | 5/5 (100%) | 0/5 (0%) | 0/5 (0%) |
| 371-clone 11/NNB 510 | 5/5 (100%) | 0/5 (0%) | 0/5 (0%) |
| 510-clone 11/NNB 360 | 2/5 (40%) | 3/5 (60%) | 0/5 (0%) |
| 510-clone 11/NNB 371 | 0/5 (0%) | 4/5 (80%) | 1/5 (20%) |

Replicates of two double mutant variants, M360P/T371A and M360P/Y510I, were identified during screening (FIGS. 7A and 7B). Our goal for this initial ACE2 activity modification effort was to isolate improved variants, as opposed to the best possible variant, from the double mutant libraries. As such, combined with the apparent superiority of the leading double mutants relative to wild type ACE2 (FIGS. 7A and 7B) this isolation of duplicate clones motivated us to proceed with soluble expression and purification of the collection of variants regardless of the number of mutant library clones screened having been below that required to achieve complete library coverage.

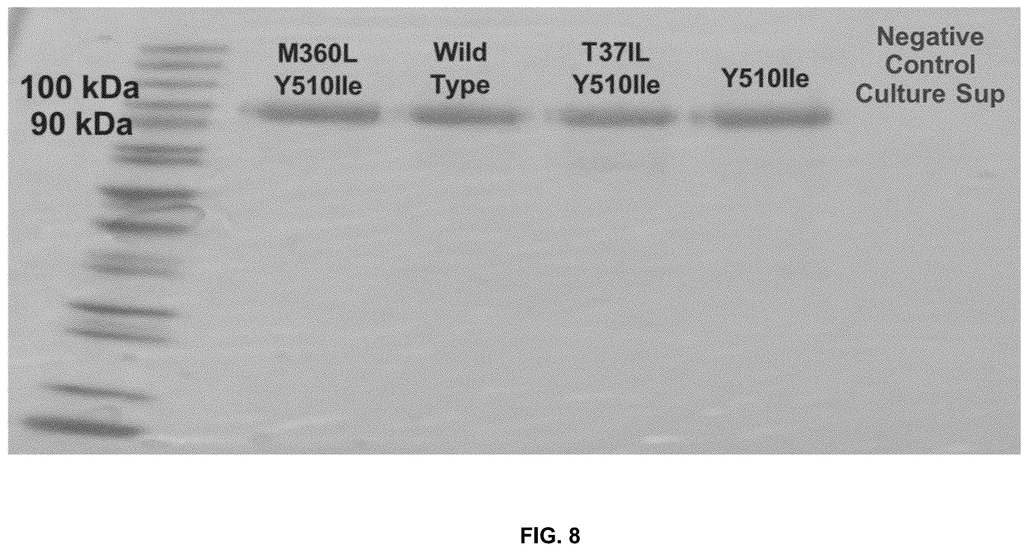

Three of the leading double mutants, where difficulties were encountered in building the M360P/T371S construct, as well as the M360P and Y510I single mutants were cloned into a soluble secretion vector [30] with C-terminal His₈ tags. The native human ACE2 gene, rather than the S. cerevisiae codon-optimized gene used in ACE2 yeast surface display plasmids, was used as the template for ACE2 mutant gene construction. Plasmids were transiently transfected into adherent T-293 human embryonic kidney cells and recombinant ACE2 proteins were purified from concentrated culture supernatants by Ni-NTA chromatography. As shown in the SDS-PAGE gel of FIG. 8, greater than 90% pure protein was obtained for both wild type and mutant ACE2s. The post-purification yield of recombinant ACE2 proteins, as determined by Bradford assay, was approximately 10 mg/L HEK culture supernatant and was nearly identical for wild type and all variant ACE2s.

Initial evaluation of ACE2 variant activity toward Ang-II and Apelin-13 in single substrate hydrolysis assays revealed that the M360P and M360P/Y510I mutants, regardless of their desirable performance as yeast-displayed proteins, had hydrolytic activity less than ten percent of wild type on an equimolar basis. The other three ACE2 mutants: Y510I, M360L/Y510I and T371L/Y510I were evaluated, along with wild type ACE2, in single substrate initial rates experiments to determine kinetic parameters, i.e., k_{cat} and Kₘ, for hydrolysis of Ang-II and Apelin-13.

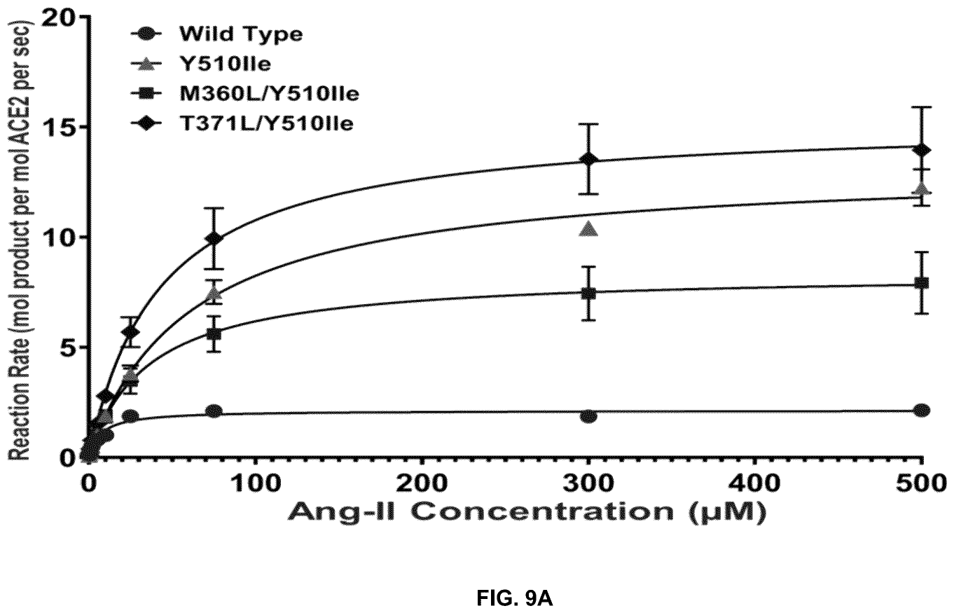

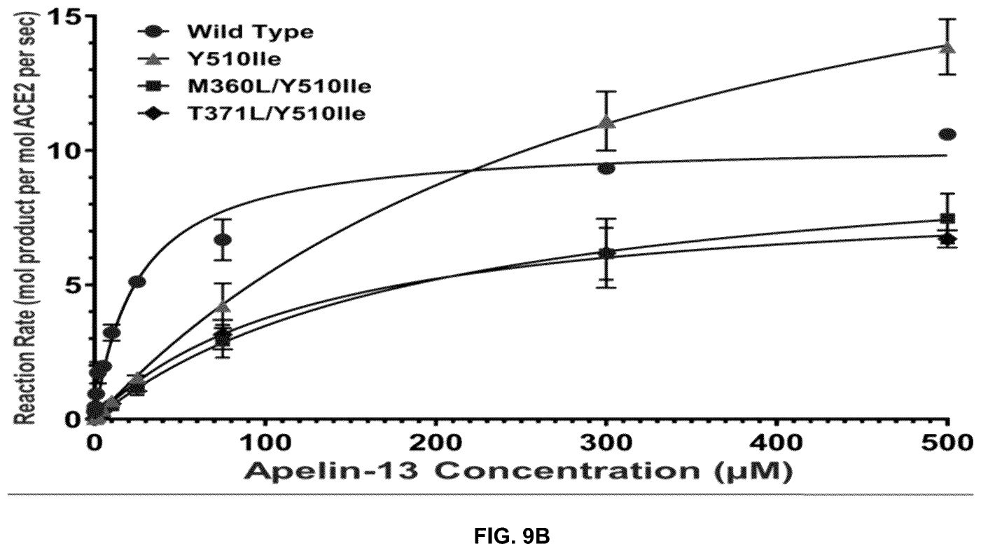

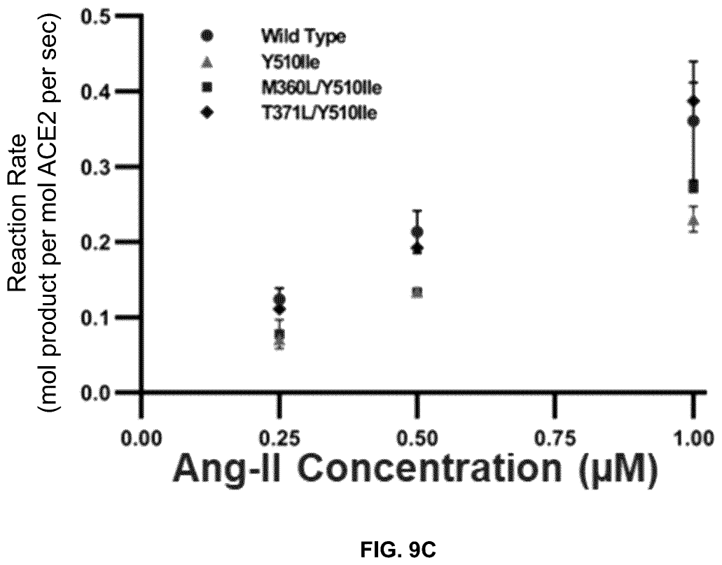

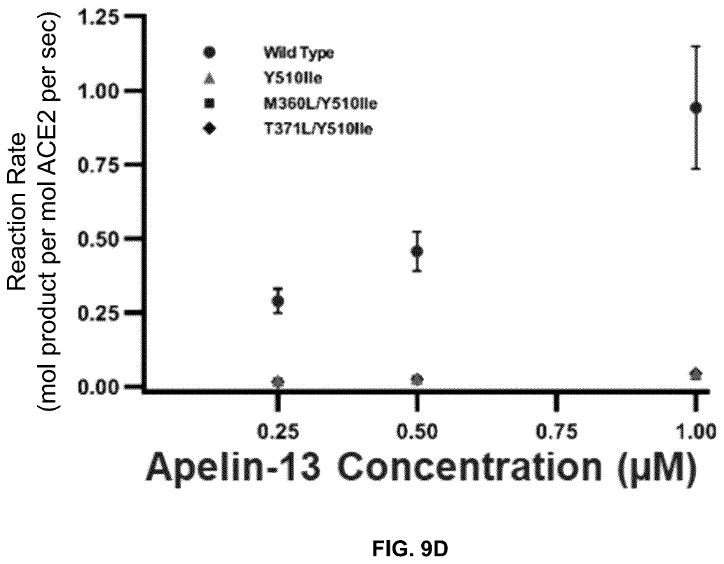

As shown in FIGS. 9A-9D, all of the mutants hydrolyze Ang-II at rates ˜ 50% or greater than those for wild type ACE2 at the lowest, and thus most physiologically relevant given levels of Ang-II found in blood and tissues [21-24], ACE2 concentrations evaluated, i.e., 0.25 and 0.5 μM. Mutant catalytic rates become greater than those for wild type ACE2 as Ang-II concentration rises above 0.5 μM. These observations are reflected in the kinetic parameter values of Table 3; k_{cat} values for ACE2 mutants are increased up to seven-fold relative to wild type and are coupled with Kₘ values that are between five- and nine-fold greater than wild type.

TABLE 3
Ang-II and Apelin-13 hydrolysis kinetic parameters for wild type and
mutant ACE2s. Parameter estimates obtained by using GraphPad Prism
software to fit initial rates data that appear in FIGS. 9A-9D.

| ACE2 | Wild Type | Y510I | M360L/Y510I | T371L/Y510I |
|---|---|---|---|---|
| Ang-II k_{cat} (s⁻¹) | 2.1 +/− 0.2 | 13.2 +/− 0.7 | 8.4 +/− 0.1 | 15.3 +/− 0.3 |
| Ang-II Kₘ (μM) | 7.2 +/− 2.8 | 60.4 +/− 10.7 | 35.9 +/− 2.1 | 42.1 +/− 2.5 |
| Ang-II k_{cat}/Kₘ (s⁻¹*M⁻¹) | 2.9*10⁵ | 2.2*10⁵ | 2.3*10⁵ | 3.6*10⁵ |
| Apelin-13 k_{cat} (s⁻¹) | 10.3 +/− 1.2 | 23.1 +/− 0.6 | 10.4 +/− 0.3 | 8.6 +/− 0.3 |
| Apelin-13 Kₘ (μM) | 25 +/− 10 | 330 +/− 20 | 200 +/− 12 | 130 +/− 15 |
| Apelin-13 k_{cat}/Kₘ (s⁻¹*M⁻¹) | 4.1*10⁵ | 7.0*10⁴ | 5.2*10⁴ | 6.7*10⁴ |

The Apelin-13 initial rates data of FIGS. 9A-9D show that all of the mutants have substantially less activity toward this substrate than does wild type ACE2. For sub-micromolar concentrations of this off-target substrate activity relative to wild type is reduced between five- and ten-fold for all three mutants. Although the activity difference narrows with increasing Apelin-13 concentration, even at 500 μM substrate the Y510I single mutant is the only ACE2 variant hydrolyzing Apelin-13 at a rate matching or exceeding that for wild type ACE2. The mutant ACE2 Apelin-13 Kₘ values of Table 3, which are between five and thirteen times that for wild type, illustrate the above described Apelin-13 hydrolysis behavior.

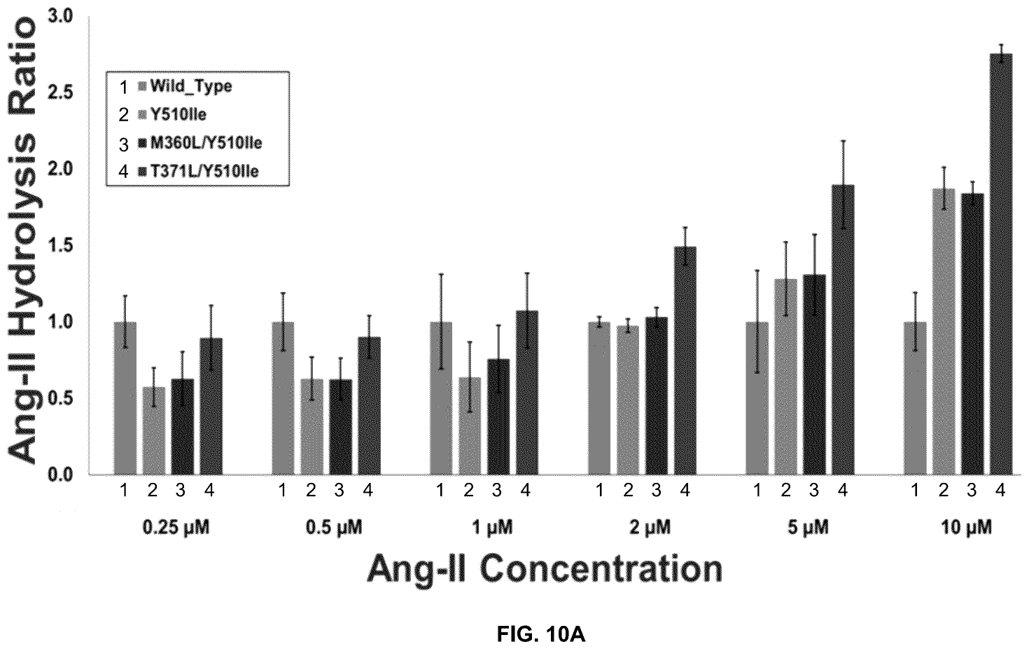

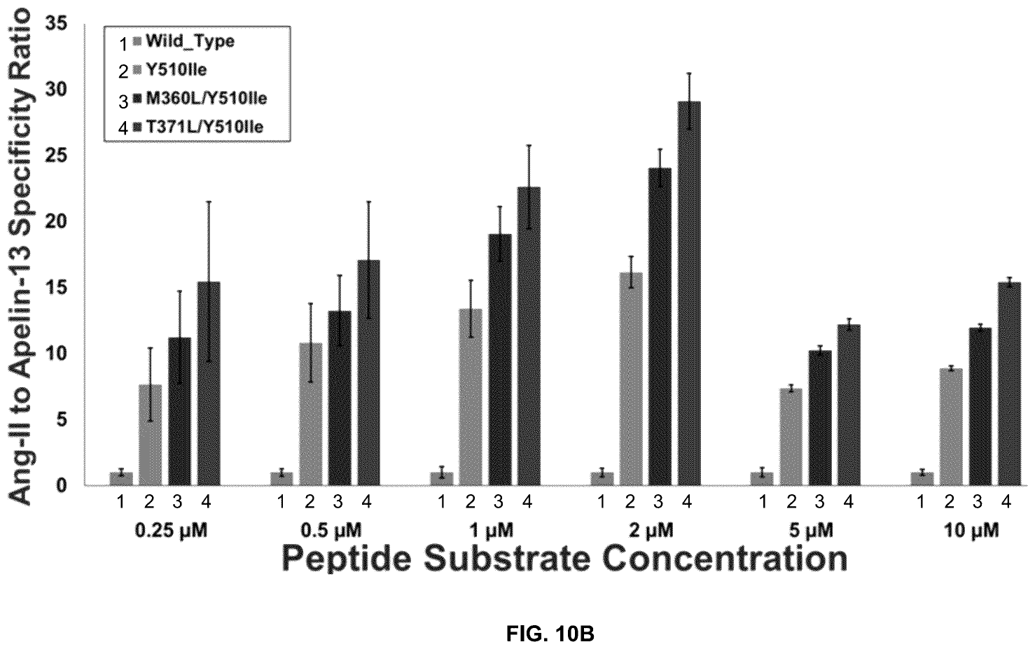

The bar plots of FIGS. 10A and 10B incorporate the single substrate hydrolysis assay results of FIGS. 9A-9D in showing that all of the mutant ACE2s have Ang-II:Apelin-13 hydrolysis ratios that are greater than wild type regardless of substrate concentration. Although the plots do not include the FIGS. 9A-9D data for substrate concentrations greater than 10 μM, increased Ang-II:Apelin-13 hydrolysis ratios are greater than wild type for all mutant ACE2s at every substrate concentration assayed in the initial rates plots of FIGS. 9A-9D. As alluded to above, these increased hydrolysis ratios are accompanied by Ang-II hydrolysis rates that range from approximately fifty percent of to seven times those for wild type ACE2 over the range of Ang-II concentrations assayed. The T371L/Y510I double mutant is especially noteworthy in that its Ang-II hydrolysis rates are equal to or greater than wild type across all Ang-II concentrations assayed in the initial rates studies of FIGS. 9A-9D. Additionally, its ratio of Ang-II:Apelin-13 hydrolytic activity is increased greater than ten-fold for all of the substrate concentrations for which initial rates were measured.

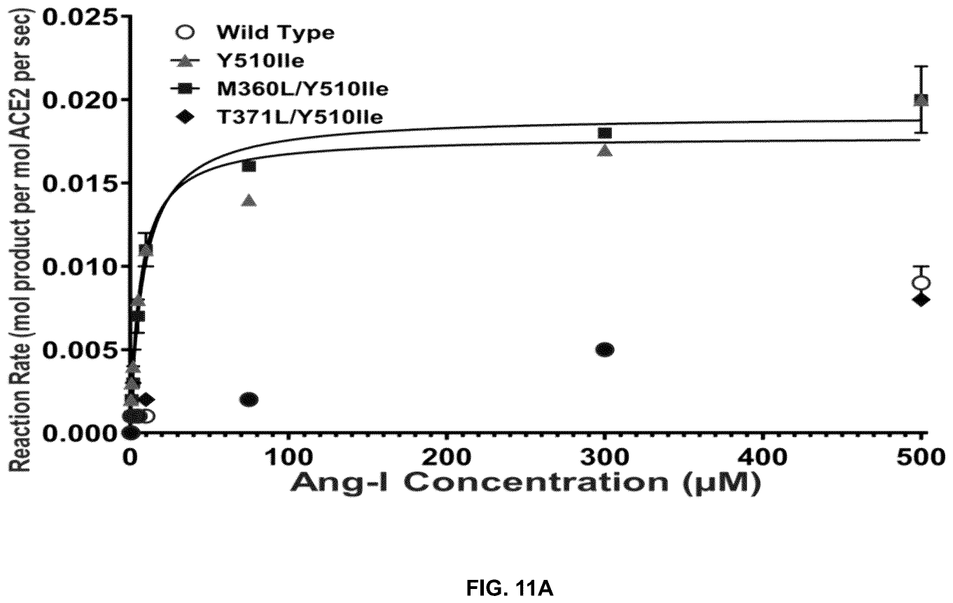

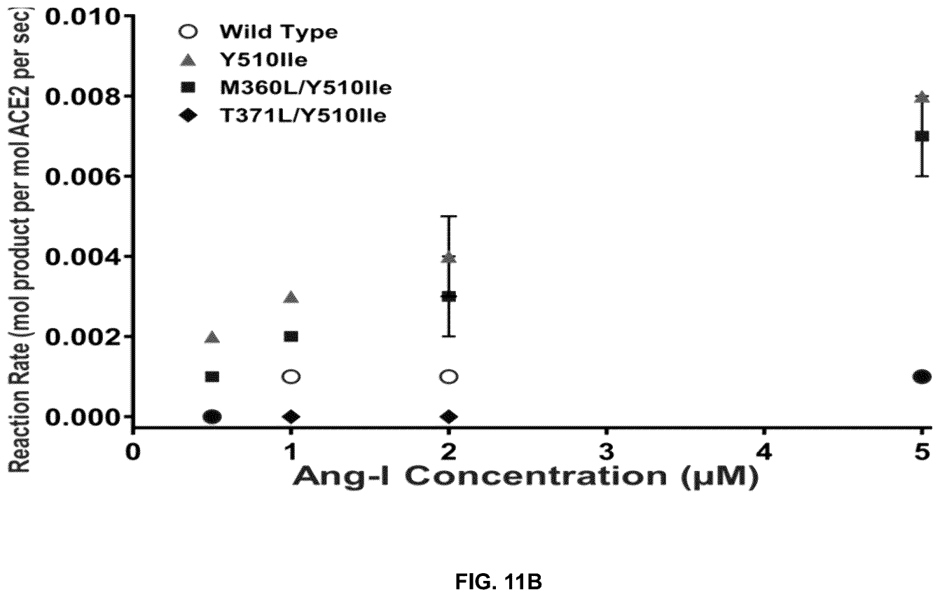

Supplementing the analyses of Ang-II and Apelin-13 hydrolytic activity with characterization of mutant ACE2 activity toward off-target peptides known to be hydrolyzed by wild type ACE2 in vivo provides useful insight regarding whether the screening procedure, which focused on Ang-II hydrolytic activity and the ratio of Ang-II:Apelin-13 activity, can lead to the isolation of mutants that may possess increased propensity to cause undesirable side effects when administered as therapeutics. The initial rates data for wild type and mutant ACE2 hydrolysis of Ang-I that is presented in FIGS. 11A and 11B show that the T371L/Y510I mutant, which has the most desirable activity profile in terms of Ang-II and Apelin-13 hydrolysis, hydrolyzes Ang-I at rates that are statistically identical to wild type across the entire range of assayed Ang-I concentrations. This outcome suggests that the ACE2 mutant library screening procedure employed can identify variants that are both improved with respect to Ang-II and Apelin-13 hydrolysis profiles and free of increased activity toward other ACE2 substrates.

In contrast to the T371L/Y510I mutant, the Y510I and M360L/Y510I mutants possess elevated hydrolytic activity toward Ang-I (FIGS. 11A and 11B). Hydrolytic rates for these enzymes, which behave identically, are increased between approximately three- and ten-fold relative to wild type over the evaluated Ang-I concentration range. We posit that increasing Ang-II activity concurrent with decreasing, or not altering, ACE2 activity toward other substrates is the ideal approach for creating variants that beneficially act on the Ang-II/ATR/MasR biological axis. Given however, that Ang-I is an Ang-II precursor and thus reducing levels of the former could bring about salutary decreases in concentrations of the latter, elevated activity toward Ang-I does not necessarily hinder an ACE2 variant's therapeutic utility in the context of Ang-II/ATR/MasR. The fact that ACE2 hydrolyzes Ang-I to Ang1-9, a nonapeptide with sequence DRVYIFPFH (residues 1-9 of SEQ ID NO:3) that is further hydrolyzed to Ang1-7 absent any hydrolysis to Ang-II in vivo [31-32], further speaks to the possibility of ACE2 therapeutic efficacy being enhanced by increased activity toward Ang-I.

Regardless of the highest Ang-I concentration evaluated, i.e., 500 μM, being well above the concentrations of this peptide that are encountered in vivo it was below the level needed to approach the maximum hydrolysis rate for the T371L/Y510I and wild type ACE2s. This outcome, which reflects high Kₘ values, precluded accurate determination of Ang-I hydrolysis kinetic parameters for these two enzymes. Conversely, initial rates data for the Y510I and M360L/Y510I were compatible with accurate kinetic parameter estimation; these parameter values appear in Table 4. All four of the ACE2s have Ang-I hydrolysis rates that are reduced at least 100-fold relative to those measured for identical concentrations of Ang-II over the entire assay range. This outcome is in accord with the published observation that the Ang-I hydrolysis k_{cat} value for wild type ACE2 is approximately one-hundred times less than that for hydrolysis of Ang-II [13].

TABLE 4
Ang-I hydrolysis kinetic parameters for mutant ACE2s. Parameter
estimates obtained by fitting initial rates data that appears
in FIGS. 11A and 11B. N/D denotes not determined due to
the maximum Ang-I concentration assayed, i.e., 500 μM,
being well below the concentration needed to reach the enzyme's
maximum hydrolysis rate; this phenomenon precludes accurate
kinetic parameter estimation.

| | Wild | | M360L/ | T371L/ |
|---|---|---|---|---|
| ACE2 | Type | Y510I | Y510I | Y510I |
| Ang-I k_{cat} | N/D | 0.018 +/− 0.02 | 0.019 +/− 0.01 | N/D |
| (s⁻¹) | | | | |
| Ang-I Kₘ (μM) | N/D | 6.4 +/− 2.9 | 8.6 +/− 2.5 | N/D |
| Ang-I k_{cat}/Kₘ | N/D | 2.8*10³ | 2.2*10³ | N/D |
| (s⁻¹*M⁻¹) | | | | |

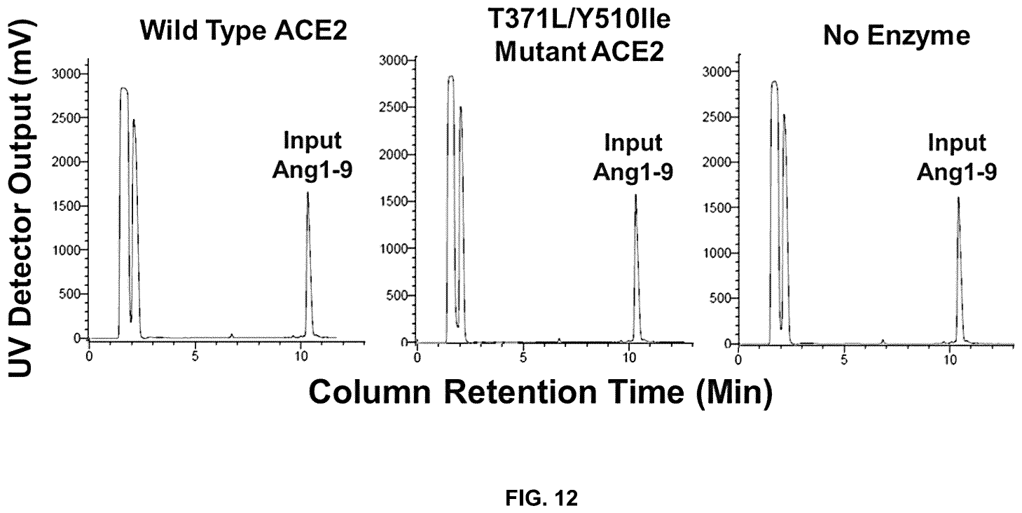

To further explore whether ACE2 mutants isolated during screening possessed increased activity toward off-target substrates assays examining the hydrolysis of the Ang1-9 nonapeptide, DRVYIFPFH (residues 1-9 of SEQ ID NO:3), which is not hydrolyzed by wild type ACE2 [13], were carried out. Regardless of incubating the ACE2 mutants with high, i.e., 100 μM and 500 μM, concentrations of Ang1-9 none of the mutant ACE2s exhibited any activity toward this peptide as determined by LC analysis (FIG. 12).

In seeking to further evaluate the specificity of the ACE2 variants, single substrate hydrolysis assays were carried out with the ACE2 natural substrate Des-Arg9-bradykinin (dR9-bk), an octapeptide with sequence RPPGFSPF (residues 1-8 of SEQ ID NO:5) [13,33]. We chose to incorporate Apelin-13, rather than dR9-bk, in ACE2 mutant library screening because the published Kₘ value of 300 μM for ACE2 hydrolysis of dR9-bk [13] is markedly higher than the single-digit micromolar Kₘ reported for Apelin-13; the high Kₘ for dR9-bk hydrolysis makes it reasonable to posit that ACE2 acts on this peptide only under rare circumstances in vivo. Further supporting the choice of Apelin-13 as an off-target peptide for use in library screening is that ACE2 hydrolysis of dR9-bK is believed to have salubrious anti-inflammatory effects [33] whereas Apelin-13 hydrolysis is viewed as a potentially pathological phenomenon [5].

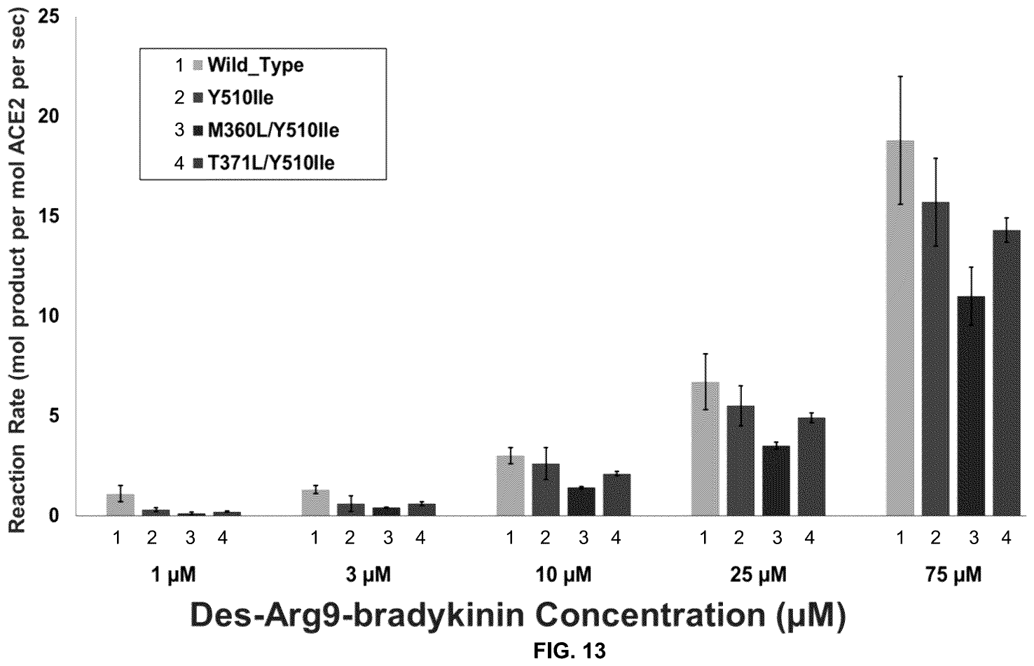

The bar plots of FIG. 13 show dR9-bk initial hydrolysis rates for the wild type and mutant ACE2s. Determination of whether mutant ACE2s had increased activity toward this substrate was efficiently achieved by assaying hydrolysis at five dR9-bk concentrations spanning from 1 μM to 75 μM. This assay range, rather than one encompassing higher substrate concentrations, was chosen on the basis of LC analysis detection limits and the low concentration, i.e., subnanomolar, of dR9-bk found in human blood [34,35]. dR9-bk hydrolysis rates for all three mutants were equal to or less than wild type across all assayed substrate concentrations. This outcome aligns with the above observations of mutant ACE2 activities toward Ang-I and Ang1-9 in suggesting that ACE2's activity profile with respect to Ang-II and Apelin-13 hydrolysis can be enhanced without concomitant compromise of specificity toward off-target substrates other than Apelin-13.

To further compare the Ang-II and Apelin-13 hydrolysis profiles for the wild type and mutant ACE2s multiplex peptide substrate hydrolysis assays, in which Ang-I, and Ang-II and Apelin-13 were simultaneously present as was the case for ACE2 mutant library screening, were carried out. Given the desire to use the assay results for assessing the therapeutic potential of the mutant ACE2s, peptide substrates were added over a concentration range, i.e., 100 nM-3 μM, commensurate with the lower detection limit for LC peptide product quantification. These assay conditions provide the closest possible approach to the substrate concentrations, i.e., between approximately 50 μM and single-digit nM, encountered in human blood and tissues [21-24]. Notable here is that extending reaction times beyond the 60-minute interval employed will increase the LC detection limit by way of increasing peptide substrate conversion but can skew ACE2 comparisons due to substrate concentrations becoming increasingly unequal across different ACE2 reactions as hydrolysis proceeds [13]. Additionally, assay reaction conditions in which substrate is appreciably depleted over time do not mirror the constant substrate concentration conditions that typically exist in vivo.

Concentration of ACE2 is an additional important consideration in seeking to maximize the utility of multiplex substrate assays for assessing the potential of mutant ACE2s as injected therapeutics. Pharmacokinetic studies with recombinant human ACE2 [36] found that immediately after injection the concentration of ACE2 in human plasma ranges from 10-100 nM depending upon dosage. ACE2 circulation half-life, which was also dose dependent, varied from 2-3.5 hours. Given these measurements, for treatment regimens featuring daily, or less frequent, ACE2 injections the majority of time is marked by ACE2 being present in the patient's bloodstream at sub-nanomolar concentrations. Given this observation, multiplex substrate hydrolysis assays were executed with 700 μM ACE2; this concentration was used in Ang-II single substrate hydrolysis assays. Additional multiplex assays featuring 250 μM ACE2 were performed to facilitate determination of how ACE2 concentration impacts hydrolytic activity and specificity.

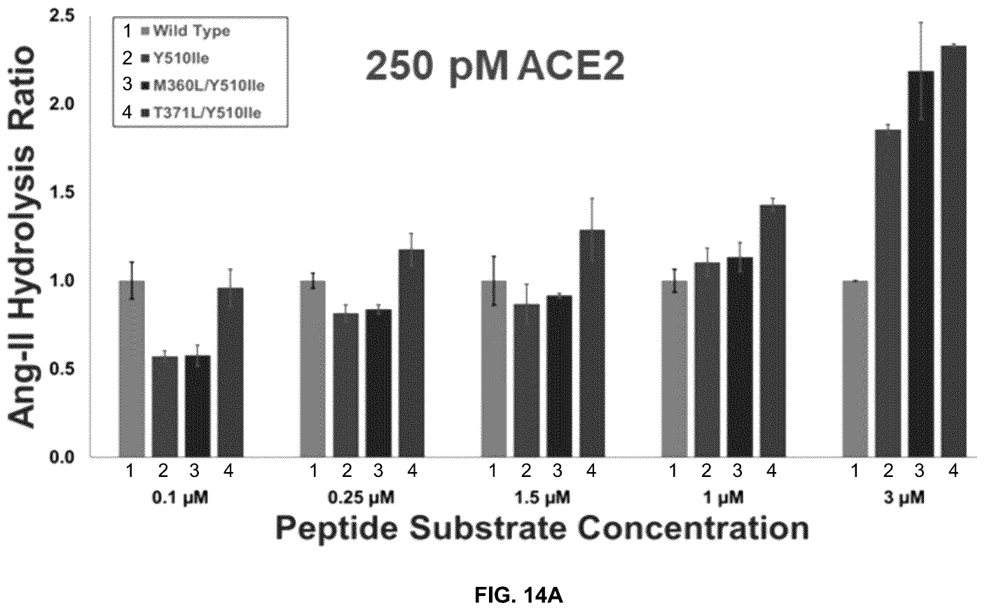

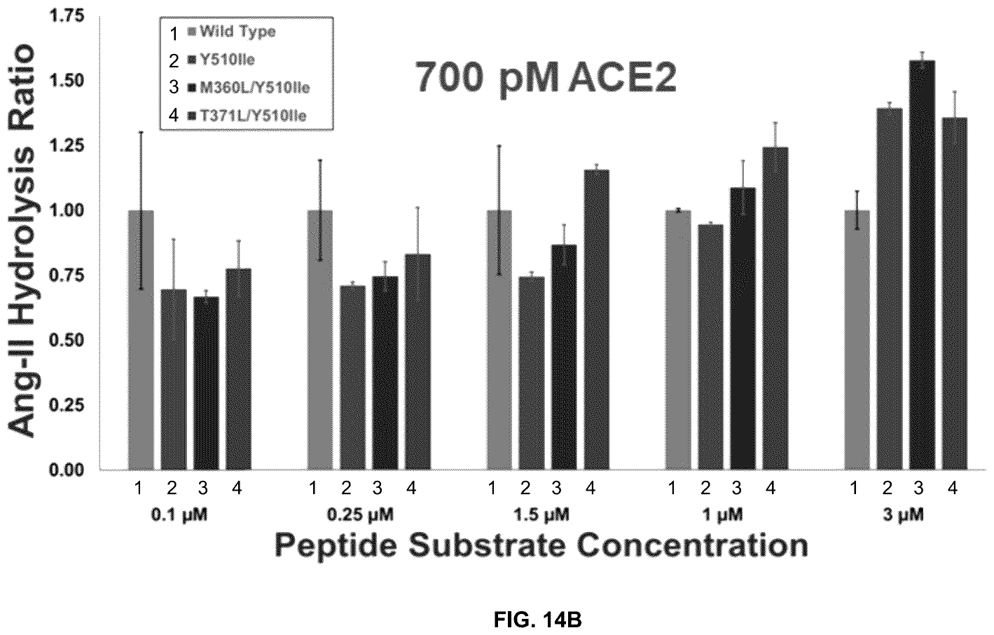

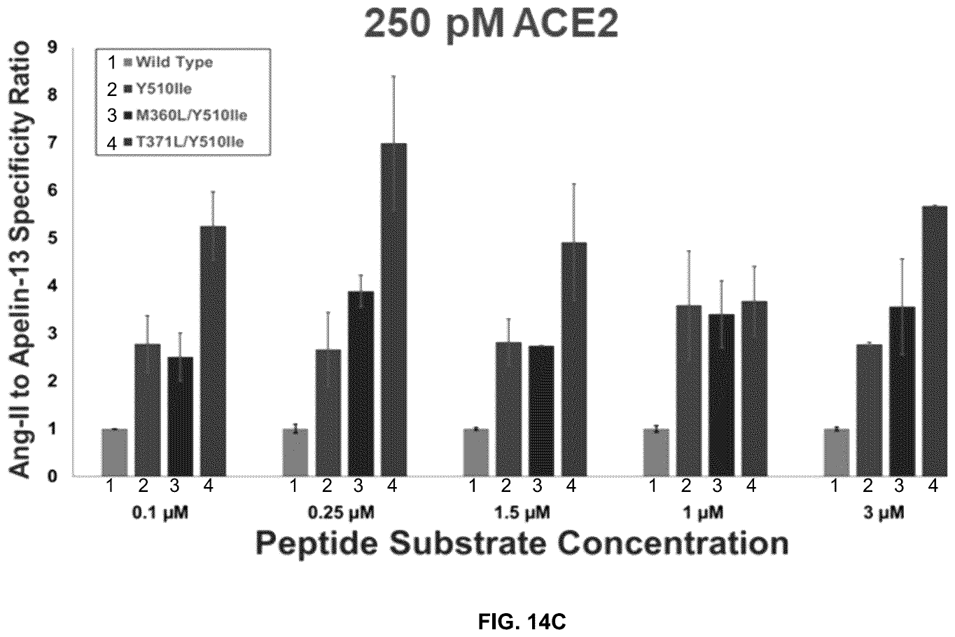

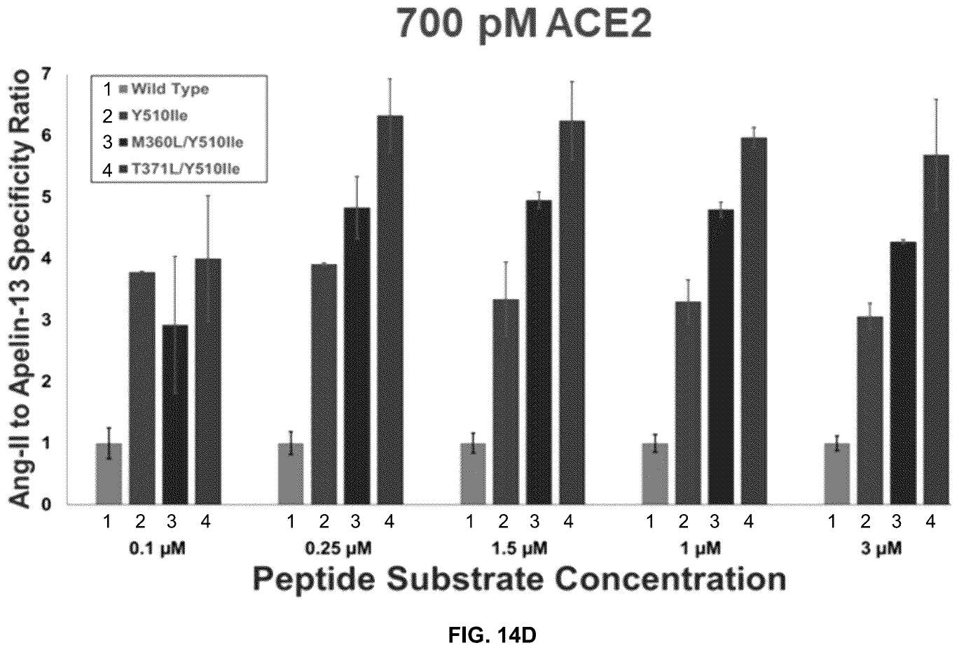

As shown in FIGS. 14A-14D, regardless of substrate or ACE2 protein concentration the mutant ACE2s hydrolyze at least half as much Ang-II as does the wild type enzyme. The T371L/Y510I double mutant, which had the most desirable activity and specificity profile in single substrate hydrolysis assays, has Ang-II hydrolysis activity that is equal to or greater than, with a greater than two-fold increase for one of the assay conditions, wild type in all of the multiplex hydrolysis reactions. The observed increases in or retention of Ang-II hydrolytic activity for the ACE2 mutants were accompanied by marked increases in Ang-II:Apelin-13 hydrolysis ratio. Irrespective of ACE2 and substrate concentration this ratio was elevated at least three-fold for all mutant ACE2s. The T371L/Y510I mutant ACE2 was the best-performing mutant in this regard with a five-fold or greater increase in specificity ratio for the majority of assay conditions.

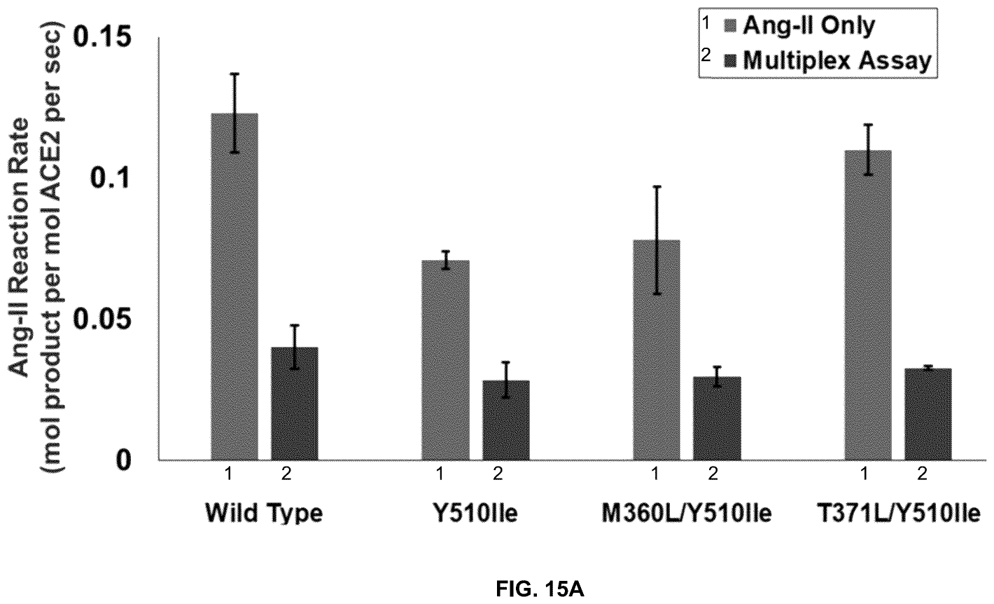

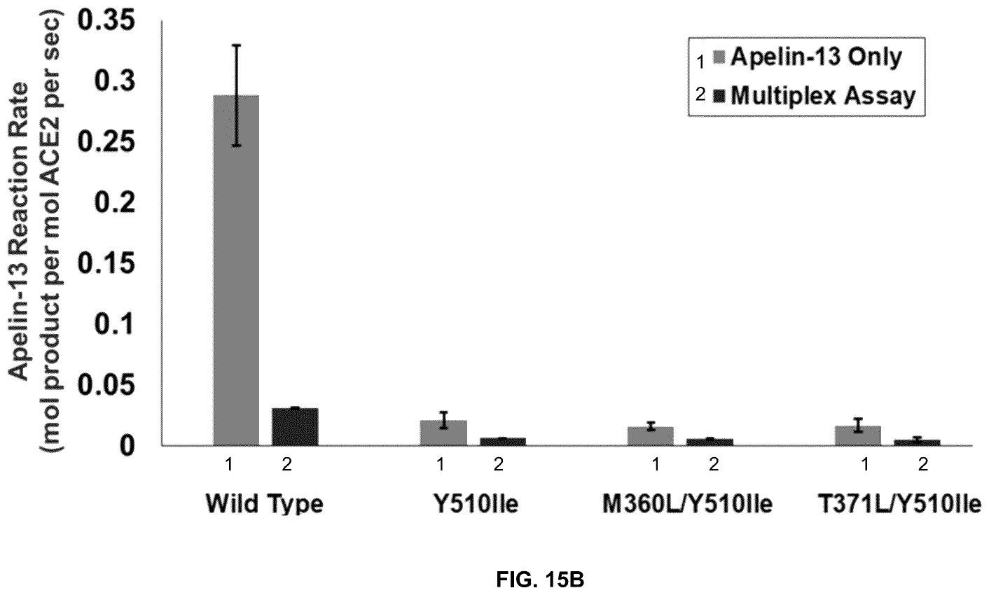

In addition to assessing the substrate hydrolysis properties of the mutant enzymes relative to wild type, these multiplex assays provide insight regarding how hydrolysis rates are impacted by the presence of multiple substrates. FIGS. 15A and 15B compare the Ang-II and Apelin-13 hydrolysis rates across single and multiplex substrate hydrolysis assay for the lowest substrate concentration, i.e., 0.25 μM, at which data was obtained for both of the single and multiplex assay formats. As anticipated, the presence of multiple substrates reduces hydrolysis rates for both Ang-II and Apelin-13. Ang-II hydrolysis rates are reduced between two- and three-fold while Apelin-13 hydrolysis rates are reduced by factors ranging from three to nine. In light of the LC chromatogram results of FIGS. 4A-4D, it is likely that the presence of Ang-I, added to multiplex assays at concentrations equal to those for Ang-II and Apelin-13, is a substantial contributor to the observed reduction in Ang-II and Apelin-13 hydrolysis rates.

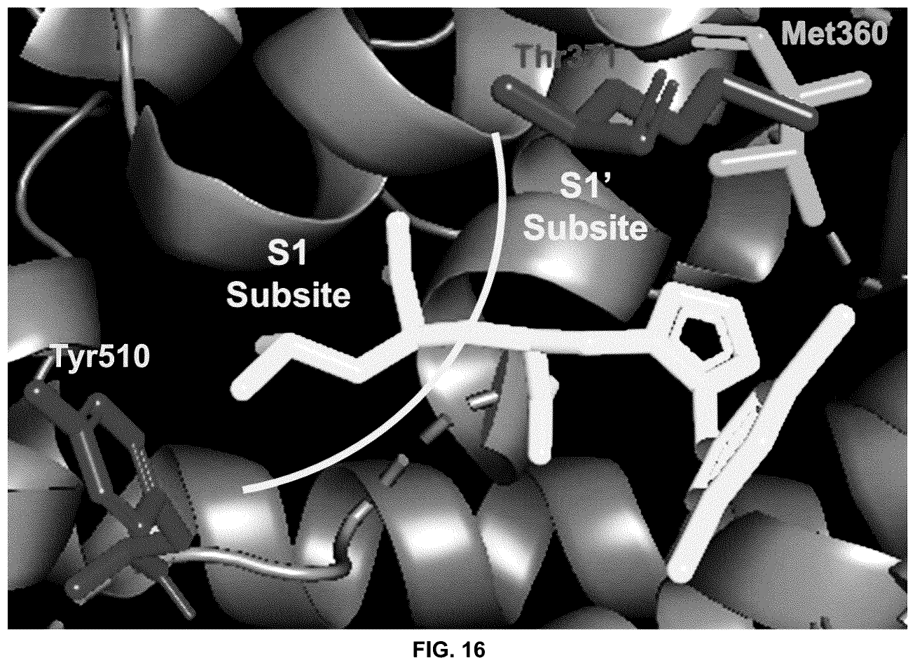

Examination of the crystal structure of ACE2 complexed with a small molecule inhibitor [28, 37] allows postulation of mechanisms by which the M360L, T371L and Y510I mutations influence ACE2's hydrolytic activity and specificity. Y510 resides in the S1 subsite of ACE2's substrate binding pocket (FIG. 16). ACE2 substrates [37], with the exception of the slowly-hydrolyzed Ang-I peptide, feature residues with small sidechains, i.e., Pro or Leu, at the corresponding P1 position; both Ang-II and Apelin-13 carry Pro at this position. Changing Tyr to Ile may increase Ang-II hydrolysis by enlarging the S1 subsite and/or creating favorable hydrophobic interactions between ACE2 and the Ang-II Pro sidechain. With respect to the ACE2 Y510I mutation's impact in terms of increasing ACE2 specificity toward Ang-II, Ang-II and Apelin feature an identical Pro residue at the P1 position but carry different amino acids at all of the other upstream peptide residue positions. As such, it is possible that interactions, which would likely be different in the context of Ang-II than in the context of Apelin-13, between the enzyme and substrate at these positions affect the way in which the substrate Pro is oriented within ACE2's S1 subsite.

The sidechains of M360 and T371 line the interior of ACE2's S1′ subsite. This subsite accommodates the C-terminal Phe of both Ang-II and Apelin-13. The similarity in sidechain size among Met, Leu, and Thr suggests that hydrophobic effects contribute to the observed impacts of the M360L and T371L substitutions on Ang-II hydrolysis rate and specificity toward Ang-II relative to Apelin-13. The fact that Ang-II and Apelin-13 are identical at the P1′ position motivates the hypothesis that substrate positioning within ACE2's binding pocket, and thus substrate hydrolysis rate and the concentration dependence thereof, is affected by enzyme-substrate interactions that occur between ACE2 residues and substrate amino acids upstream of the P1′ position. As alluded to above, such interactions are likely to be distinct across the Ang-II and Apelin-13 peptides and thus could cause mutations in the ACE2 substrate binding pocket, e.g., M360L and T371L, to have different effects on ACE2 activity toward these respective substrates.

### Discussion

The results presented here show that the LC screening approach we have developed enables isolation of ACE2 variants with potential to outperform wild type ACE2 as protein therapeutics. This outcome can have an important impact in both advancing current ACE2 therapeutic initiatives, such as treating respiratory distress associated with traumatic injuries or viral infection [6], and giving rise to new ACE2 biomedical applications such as using antibody-ACE2 fusion proteins [38] to achieve localized modulation of ATR and MasR activity and employing peptidase-peptide orthogonal pairs [39] to enable targeted prodrug activation.

Although the mutant ACE2s described here show potential as superior alternatives to wild type ACE2 for beneficially modulating ATR and MasR activity in vivo there are arenas in which mutant properties can be further enhanced. The most prominent of these is reduction of Kₘ for hydrolysis of the Ang-II target peptide; whereas all of the mutants consistently hydrolyze Ang-II faster than the wild type enzyme at substrate concentrations above one μM their hydrolysis rates are at parity with or below that of wild type ACE2 for substrate concentrations approaching the physiological Ang-II concentration range of 10-300 μM [22-24]; this phenomenon is reflected in the increases in both k_{cat} and Kₘ shown in Table 3. The objective of reducing ACE2 mutant Kₘ while concomitantly maintaining or increasing k_{cat} can be realized through additional screening of existing ACE2 mutant libraries and/or constructing triple mutant libraries by carrying out site-directed mutagenesis on the M360L/Y510I and T371LY510I double mutants.

With respect to the former, for this initial pursuit of enhanced ACE2 mutants the site-directed single mutant libraries were not exhaustively screened as our objective was to verify the ability to identify improved ACE2 variants rather than to isolate the very best variants that could be obtained within the boundaries of the ACE2 positions targeted for mutagenesis. Having now demonstrated both the utility of our ACE2 mutant library screening methodology and retention of improvements in mutant activity and specificity profiles after moving from the yeast-displayed to soluble, purified ACE2 protein format, the uncertainty regarding ACE2 mutant library screening outcomes has been markedly reduced. As such, there is motivation to expand the scale of site-directed single mutant library screening to a level, i.e., approximately ninety clones per library [29], that corresponds to greater than 95% probability of identifying the best mutant in each library. This screening process can be streamlined by our having identified the ACE2 binding pocket positions that are most tolerant to substitution; as shown in Table 1 our observations in this regard motivate an emphasis the six respective single site-directed libraries mutated at positions E145, N149, M360, K363, T371 and Y510. In a similar vein, further enhancement of the current leading ACE2 double mutants, i.e., M360L/Y510I and T371L/Y510I, can be efficiently pursued by way of constructing and screening mutant progeny libraries carrying NNB codons at the positions in the above list of six that currently feature wild type residues in the background of these two ACE2 variants.

A comparison of the substrate specificity results presented in FIGS. 10 and 14 underscores the value of evaluating ACE2 mutants in multiplex peptide substrate hydrolysis assays. In particular, the presence of multiple substrates reduces Ang-II:Apelin-13 hydrolysis ratios relative to those calculated based on hydrolysis rates for single substrate hydrolysis assays. This compression of substrate hydrolysis ratios indicates that in addition to being more representative of in vivo conditions than is the case for single substrate assays, multiplex substrate assays provide a more stringent test of mutant ACE2 substrate specificity relative to wild type and parental mutant ACE2 enzymes.

Insight regarding the origins of the reduction in mutant ACE2 multiplex assay Ang-II:Apelin-13 hydrolysis ratios relative to the values calculated for single substrate assays can be obtained by noting that mutant ACE2 single substrate assay Ang-II hydrolysis rates are between three- and ten-fold greater than single substrate assay Apelin-13 hydrolysis rates for single peptide reactions featuring substrate concentrations that fall within the range of concentrations used in multiplex assays. The difference in hydrolysis rates indicates that time during which the ACE2 active site is occupied by Ang-I or Apelin-13 is greater relative to the time required for an Ang-II hydrolysis event than is the time of active site occupation by Ang-I or Ang-II relative to the time needed for an Apelin-13 hydrolysis event. As such, the phenomenon of shared active site occupancy leads to a greater relative reduction in hydrolysis rate of Ang-II than in hydrolysis rate of Apelin-13 and a corresponding decrease in multiplex assay Ang-II:Apelin-13 hydrolysis rate ratios when compared to ratios calculated based on rates measured in single substrate assays.

The stark difference, with the soluble proteins having less than 10% of wild type activity toward Ang-II and Apelin-13, in hydrolytic activity relative to wild type ACE2 for the M360P and M360P/Y510I mutants across yeast-displayed and soluble, purified formats illustrates the need to consider clonal diversity when choosing a group of variants for soluble expression following yeast-displayed ACE2 library screening; had all of the ACE2 variants chosen for soluble expression after double mutant library screening carried the M360P mutation the desirable effects of the T371L and Y510I substitutions in the context of soluble, purified ACE2s would have been masked by the markedly reduced activity associated with the M360P mutation. The low level of hydrolytic activity observed for soluble ACE2 variants carrying the M360P substitution is not without precedent as display on the yeast surface has been observed to stabilize biologically active conformations of mutant proteins that are prone to taking on inactive conformations when free in solution [40-41].

The isolation of ACE2 variants with enhanced activity and specificity profiles described in these examples establishes a foundation for using yeast display-based screening to both develop an additional cohort of ACE2 variants that act on the Ang-II/ATR/MasR biological axis and create ACE2 mutants that exert beneficial effects in the context of other medically relevant signaling interaction networks. There is cause for optimism regarding the impact that the methods developed here, along with possible extrapolations thereof such as microfluidics droplet-based screening for ACE2 activity and specificity [27], can have in enabling ACE2 to realize its full potential as an agent for treating and preventing a wide range of health conditions.

### Materials and Methods

### ACE2 Library Generation and Screening

Residues 18-615 of the human (UniProt Q9BYF1) ACE2 genes were synthesized as a yeast codon-optimized gBlock (SEQ ID NO:12; Integrated DNA Technologies, Coralville, IA) and ligated into the NheI and MluI sites of yeast display vector VLRB.2D-aga2 (provided by Dane Wittrup, MIT); this vector fuses the aga2 protein to the C-terminus of ACE2 (FIG. 2). SEQ ID NO:12 is:

(SEQ ID NO: 12)
CAATCTACCATCGAGGAGCAAGCTAAGACTTTCCTAGATAAGTTCAACCA
TGAGGCCGAAGACTTATTTTACCAGAGCTCACTAGCAAGCTGGAACTATA
ACACTAACATAACAGAGGAGAATGTGCAAAACATGAATAATGCTGGCGAT
AAGTGGAGTGCCTTCTTGAAGGAGCAAAGTACCCTTGCCCAGATGTATCC
TCTACAGGAAATACAAAACCTGACGGTTAAGCTACAATTACAAGCTCTGC
AACAAAATGGGTCAAGTGTTTTGTCCGAAGACAAGAGTAAAAGGCTTAAT
ACGATCCTAAACACGATGAGCACCATCTACTCTACGGGGAAGGTCTGTAA
CCCCGACAACCCTCAAGAGTGTCTGCTGCTGGAACCAGGTTTGAACGAGA
TCATGGCCAACTCATTGGATTATAATGAGCGTCTGTGGGCCTGGGAATCC
TGGAGGTCCGAGGTGGGGAAACAACTTAGACCCCTTTACGAGGAGTACGT
TGTTCTAAAGAACGAAATGGCAAGGGCTAATCACTACGAGGATTACGGCG
ACTACTGGAGAGGGGACTATGAAGTAAACGGTGTAGACGGCTACGATTAT
AGCAGGGGCCAGCTGATCGAAGATGTAGAGCATACTTTCGAGGAGATTAA
GCCACTGTATGAGCATTTGCATGCATATGTAAGGGCCAAGTTAATGAACG
CTTACCCCTCTTATATATCTCCCATCGGATGTCTGCCTGCTCACTTACTT
GGGGACATGTGGGGGCGTTTCTGGACTAACTTATACTCCCTGACAGTACC
CTTTGGTCAAAAGCCGAATATAGACGTAACGGACGCTATGGTGGACCAAG
CGTGGGACGCGCAGAGAATTTTCAAGGAGGCGGAGAAGTTTTTTGTATCA
GTGGGCCTTCCGAACATGACTCAAGGATTTTGGGAGAATTCTATGCTAAC
CGACCCAGGCAACGTGCAAAAAGCAGTCTGCCACCCCACCGCATGGGATC
TGGGAAAAGGGGATTTTCGTATACTAATGTGTACAAAAGTCACGATGGAT
GATTTCTTAACGGCACACCATGAGATGGGCCATATACAGTACGACATGGC
ATATGCGGCCCAGCCTTTTCTATTGAGGAACGGGGCCAACGAGGGTTTCC
ATGAAGCGGTTGGAGAGATAATGTCACTAAGTGCAGCGACGCCCAAGCAT
CTGAAAAGCATTGGGCTGTTAAGCCCGGACTTTCAGGAAGACAACGAAAC
GGAAATTAACTTTTTGCTAAAACAGGCACTGACGATCGTTGGCACTCTAC
CCTTTACGTATATGTTAGAAAAGTGGAGGTGGATGGTTTTTAAGGGTGAA
ATTCCCAAAGACCAATGGATGAAGAAATGGTGGGAAATGAAGAGGGAAAT
TGTCGGCGTGGTTGAGCCGGTGCCTCACGACGAGACTTACTGTGACCCGG
CCTCTTTGTTCCACGTATCCAATGATTACAGCTTTATTAGGTACTACACT
CGTACGTTATATCAATTCCAGTTTCAAGAAGCGCTGTGCCAGGCAGCTAA
GCATGAAGGTCCTTTACACAAATGCGACATATCTAACTCAACTGAGGCGG
GTCAGAAACTTTTCAATATGCTAAGGCTAGGTAAATCAGAGCCATGGACT
CTAGCTTTAGAGAACGTAGTGGGTGCCAAGAACATGAACGTGAGACCACT
GCTAAACTATTTTGAACCACTGTTCACTTGGCTTAAGGATCAGAACAAGA
ATAGTTTTGTCGGATGGTCTACAGACTGGTCCCCCTATGCAGAC

Site-directed mutant libraries were constructed via overlap extension PCR and standard ligation using T4 DNA ligase (New England Biolabs, Beverly, MA). Oligonucleotide primers carrying NNB base triplets were purchased from Integrated DNA Technologies. The degenerate “B” base is comprised of a mixture of C, G and T bases while the degenerate “N” base is composed of all four nucleotide bases. Primers CDspLt (5′-GTCTTGTTGGCTATCTTCGCTG-3′ (SEQ ID NO:13)) and CDspRt (5′-GTCGTTGACAAAGAGTACG-3′ (SEQ ID NO:14)) were used as outer primers for ACE2 gene amplification reactions. Overlap extension PCR products were digested using NheI and MluI (New England Biolabs) and ligated into the yeast surface display vector VLRB.2D-aga2 digested with these same two enzymes. Overnight ligation reactions were desalted using a DNA Clean & Concentrate-5 column (Zymo Research, Orange, CA) and transformed into chemically competent NEB 5a cells (New England Biolabs).

For each transformation cells were plated onto three LB plates containing 100 μg/mL carbenicillin and after overnight incubation at 30° C. all colonies on plates for each respective transformation were scraped into 10 mL of LB+carbenicillin liquid media and grown overnight at 30° C. Total colony counts for each single site-directed mutant library ranged from 500 to 2,000; numbers considerably greater than the fewer than 50 colonies observed for a negative control ligation reaction that contained digested VLRB.2D-aga2 backbone DNA absent any ACE2 gene insert.

DNA was harvested from overnight liquid cultures using the Qiagen Spin Miniprep Kit (Qiagen, Valencia, CA) and transformed into the EBY100 yeast surface display strain [1] that had been made chemically competent using the Frozen EZ-Yeast Transformation II Kit (Zymo Research). For each single site-directed mutant library transformed yeast were plated onto three each Synthetic Dropout minus Tryptophan (SD-Trp) plates and incubated at 30° C. for two days. Individual yeast colonies were picked into 4 mL of pH 4.5 Sabouraud Dextrose Casamino Acid media (SDCAA: Components per liter-20 g dextrose, 6.7 grams yeast nitrogen base (VWR Scientific, Radnor, PA), 5 g Casamino Acids (VWR), 10.4 g sodium citrate, 7.4 g citric acid monohydrate) and grown overnight at 30° C. and 250 rpm. For induction of ACE2 display a 5 mL pH 7.4 Sabouraud Galactose Casamino Acid (SGCAA: Components per liter-8.6 g NaH₂PO*H₂O, 5.4 g Na₂HPO₄, 20 g galactose, 6.7 g yeast nitrogen base, 5 g Casamino Acids) culture was started at an optical density, as measured at 600 nm, of 0.5 and shaken overnight at 250 rpm and 20° C.

For LC determination of yeast-displayed ACE2 peptidase activity 35 μL of induced yeast culture was transferred to 500 μL of pH 7.4 Phosphate Buffered Saline (PBS) containing 0.1% (wt/vol) bovine serum albumin (BSA) and centrifuged at 4000 g for one minute. Yeast were resuspended in 400 μL of ACE2 reaction buffer (50 mM 2-(N-morpholino)ethanesulfonic acid (MES), 300 mM NaCl, 10 μM ZnCl₂, 0.02% w/v Hen Egg Lysozyme (Sigma-Aldrich, St. Louis, MO) as carrier protein, pH 6.5) containing 25 μM each Angiotensin I (Anaspec, Fremont, CA), Angiotensin II (Anaspec) and Apelin-13 (Bachem, Torrance, CA) and tumbled in microfuge tubes at 20 rpm at room temperature for 2.5 hours. Reactions were centrifuged at 9000 g for one minute to pellet yeast and supernatants withdrawn for LC analysis.

Liquid chromatography (LC) analyses were performed on a Shimadzu 2020 LC-MS apparatus operating in UV detection mode at 214 nm. Sixty-five μL of ACE2 reaction were injected onto a 3×150 mm, 100 Δ, 5 μm Polar C18 Luna Omega column (Phenomenex, Torrance, CA) using a linear gradient of 11% B to 100% B over 25 minutes with 5 minutes at final conditions and 8-minute re-equilibration. Mobile phase A consisted of 0.02% (v/v) trifluoroacetic acid in water, and mobile phase B consisted of 0.016% (v/v) trifluoroacetic acid in acetonitrile. Mobile phase flowrate was maintained at 0.3 mL/min throughout sample analysis. Hydrolysis product peaks were identified by comparison with chromatograms for reactions carried out using yeast displaying a negative control protein (horseradish peroxidase) and the area under the LC chromatogram curve, i.e., UV detector millivolts multiplied by elution timespan in seconds, was used as the metric for amount of product formed during the hydrolysis reaction.

For construction of ACE2 double mutant libraries that used leading single mutant clones, i.e., clone 360-7, 371-11, and 510-11 as described in the Results section, plasmids were rescued from 1 mL of yeast liquid culture using the Zymo Research Yeast Plasmid Miniprep II Kit. Rescued plasmids were used as templates for overlap PCR reactions carried out to introduce NNB codons, at the positions noted in the Results section, into the three respective single mutant ACE2 parent genes. Digestion and ligation of double mutant library PCR product DNA, E. coli transformation, E. coli culturing, double mutant library plasmid harvesting and EBY100 yeast transformations were carried out using procedures, as described above, identical to those for site-directed ACE2 single mutant libraries. Sequences for leading ACE2 double mutants were obtained by using the Zymo Yeast Plasmid Miniprep II kit, amplifying double mutant ACE2 genes using primers CDspLt and CDspRt, purifying the PCR products using a Zymo Clean & Concentrator-5 column and sequencing the PCR products using primers CDspLt, CDspRt and SqHmFw (5′-GGACTTTCAGGAAGACAACG-3′ (SEQ ID NO: 15)).

Soluble Expression & Purification of Wild Type andACE2 Variants

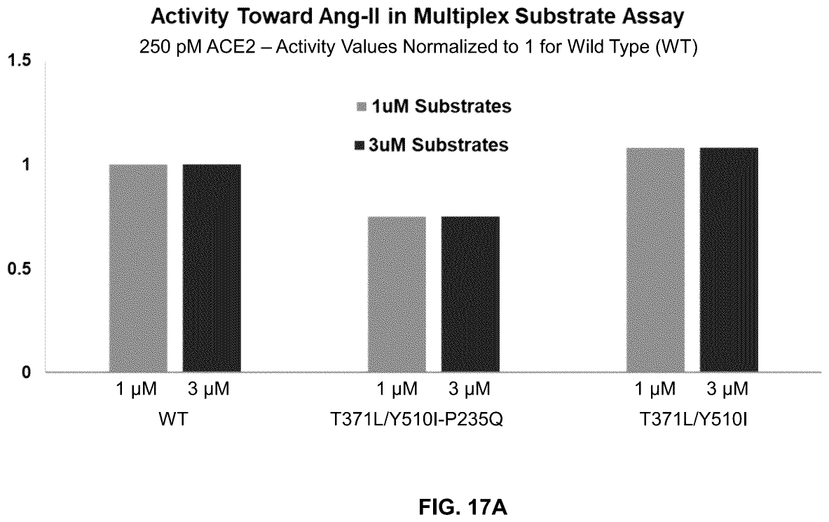

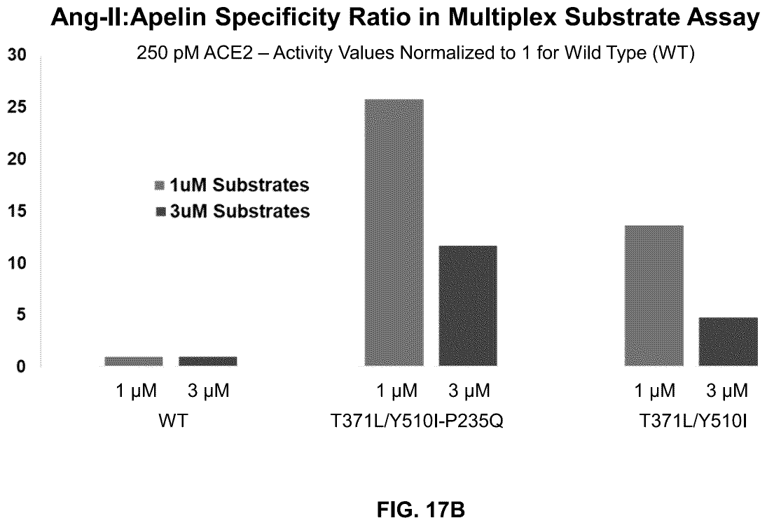

Plasmid pcDNA3-sACE2(WT)-8 his [29], which encodes human ACE2 residues 1-615 with native human codon representation and puts secretion of the gene product, with a C-terminal His8 tag, from mammalian cells under control of the cytomegalovirus CMV promoter, was used as template for creation of ACE2 genes carrying hydrolysis profile-modifying mutations identified during yeast displayed-ACE2 library screening. Mutant genes were constructed by overlap extension PCR using outer primers SqLtPck (5′-CGTGGATAGCGGTTTGACTCAC-3′ (SEQ ID NO:16)) and PckRvSq (5′-CCTACTCAGACAATGCGATGC-3′ (SEQ ID NO:17)), digested NheI-BamHI (New England Biolabs) and ligated into pcDNA3-sACE2(WT)-8 his that had been digested using these same enzymes. Overnight ligation reactions were desalted, transformed into chemically competent NEB 5α cells as above and plated onto LB plates containing 100 μg/mL carbenicillin. Individual transformants were picked into 5 mL of LB media containing 100 μg/mL carbenicillin and incubated overnight at 37° C. DNA was harvested from overnight liquid cultures using the Qiagen Spin Miniprep Kit and inserted mutant ACE2 genes were verified by sequencing using primers SqLtPck, PckRvSq and PckF260 (5′-GTTACTGATGCAATGGTGGACC-3′ (SEQ ID NO:18)). The vector expressing the T371L/Y510I ACE2 mutant further included a P235Q mutation due to a polymerase error. As shown in FIGS. 17A and 17B, the P235Q mutation does not have an appreciable effect on Ang-II activity (FIG. 17A) or Ang-II:Apelin specificity (FIG. 17B). Ang-II:Apelin specificity is improved with the T371L and Y510I mutations relative to wild type regardless of whether the P235Q mutation is present.

Human Embryonic Kidney 293T cells (Product #CRL-3216, ATCC, Manassas, VA) were grown in DMEM (ATCC) supplemented with 10% Fetal Bovine Serum (FBS), 2 mM L-glutamate (Invitrogen, Carlsbad, CA), and 1× Penicillin-Streptomycin (Invitrogen) in 100 mm tissue culture treated dishes under a 5% CO₂ atmosphere at 37° C. Upon reaching ˜ 70% confluence cells were transfected with 7 μg of ACE2 expression plasmid DNA and 10 μL of jetPRIME transfection reagent (PolyPlus, Illkirch-Graffenstaden, France) per the manufacturer's instructions. Two dishes of HEK cells were transfected for each ACE2 protein being expressed. Following the four-hour transfection interval DMEM containing DNA and transfection reagent was replaced with 10 mL of OptiMEM (Invitrogen) supplemented with 1× Pen-Strep and ACE2 expression was allowed to proceed for 40-48 hours at 37° C. under 5% CO₂.

Media was withdrawn from culture dishes, pooled for duplicate transfections, and centrifuged at 500 g for 5 minutes. Supernatants were syringe filtered using 0.22 μm polyethersulfone (PES) Whatman PuraDisc filters (Cytiva, Marlborough, MA). Supernatant concentration and buffer exchange into pH 7.4 PBS was performed using 30-kDa MWCO VivaSpin20 centrifugal concentrator units (Cytiva). Hiss-tagged ACE2 proteins were purified from concentrated, buffer exchanged supernatants. Concentrated supernatants were brought to a final volume of 1 mL by addition of pH 7.4 PBS, adjusted to an imidazole concentration of 10 mM and tumbled head-over-head at 20 rpm in microfuge tubes with 25 μL of Qiagen Ni-NTA resin for 1 h at room temperature. Resin was subsequently washed with 1 mL PBS and 1 mL PBS/15 mM imidazole prior to ACE2 elution via 15-minute room temperature incubation of resin in 140 μL of pH 7.4 PBS containing 250 mM imidazole. Eluted ACE2 proteins were buffer exchanged into 25 mM Tris-HCl, pH 7.5 containing 10 μM ZnCl₂ using Zeba Spin desalting columns (Fisher Scientific, Waltham, MA) and protein concentrations determined using the Pierce Coomassie Protein Assay kit (Fisher Scientific).

SDS-PAGE analysis of purified ACE2s was carried out using a LIfeTech Mini Gel Tank electrophoresis system. Approximately 1 μg of purified, desalted ACE2 was loaded into each well of a Novex 4-20% Tris-Glycine gel and electrophoresis was performed at 200 volts for 45 minutes. PageRuler Unstained Protein Ladder (Fisher Scientific) was used as a molecular weight standard. Gels were stained with Simply Blue SafeStain (Invitrogen) following electrophoresis.

### Activity and Specificity Profiling of Purified ACE2 Proteins

Peptide substrate hydrolysis assays were carried out at room temperature and 150 μL scale in 96-well plates using 50 mM MES, 300 mM NaCl, 10 μM ZnCl₂, 0.01% (vol/vol) Brij-35 (Sigma-Aldrich) as reaction buffer. Reactions were halted by addition of 15 μL of 1M EDTA solution, pH 8, after intervals, which ranged from three minutes to 2.5 hours, during which less than 15% of the input peptide substrate had been hydrolyzed. ACE2 concentrations added to assays for the various peptide substrates evaluated were as follows: Ang-I/1.75 nM, Ang-II/700 μM, Apelin-13/1.4 nM, des-Arg9-bradykinin/700 μM, Ang (1-9)/700 μM, Multiplex/700 μM or 250 μM. Des-Arg9-bradykinin and Ang1-9 were purchased from Anaspec and ApexBio (Houston, TX) respectively.

Moles of product formed in hydrolysis reactions used for ACE2 activity and specificity profiling were determined by comparing area under the curve for LC chromatograms corresponding to these reactions to areas under the curve observed for control reactions in which >95% input substrate conversion was achieved by incubation of substrate with 25 nM recombinant human ACE2 commercial standard (BioLegend, San Diego, CA) for between one and four hours pending peptide substrate. Elution times for peptide substrate inputs and hydrolysis products were determined by comparing chromatograms for the 25 nM ACE2 commercial standard reactions to input peptide reaction mixtures to which no ACE2 was added. LC analysis injection volumes and mobile phase gradient parameters were identical to those described above for yeast-displayed ACE2 library screening. Kinetic parameters for Ang-I, Ang-II and Apelin-13 hydrolysis were determined by fitting initial rates data using GraphPad Prism software (GraphPad, San Diego, CA). Standard deviations for ratios of hydrolysis rates that include values measured in single substrate hydrolysis assays were calculated by inserting the average of duplicate measurements and the standard deviation for that average into the equation—

$$For A = \frac{B}{C}$$

$$StDev A = A * \sqrt{{( \frac{StDevB}{R} )}^{2} + {( \frac{StDevC}{C} )}^{2}}$$

For single substrate hydrolysis rate ratios B denotes mutant ACE2 hydrolysis rate and C denotes wild type ACE2 hydrolysis rate.

For mutant ACE2:wild type Ang-II:Apelin-13 hydrolysis rate specificity ratios, B denotes mutant ACE2 Ang-II:Apelin-13 hydrolysis rate ratio and C denotes wild type ACE2 Ang-II:Apelin-13 hydrolysis rate ratio.

### REFERENCES

1. Chao G et al. Isolating and Engineering Human Antibodies Using Yeast Surface Display. Nat Protoc. 1(2):755-68 (2006).
2. Cherf G M, Cochran J R, Applications of Yeast Surface Display for Protein Engineering. Methods Mol Biol. 1319:155-75 (2015).
3. Shenoy V, Qi Y, Katovich M J, Raizada M K, ACE2, a promising therapeutic target for pulmonary hypertension. Curr Opin Pharmacol. 11(2):150-5 (2011).
4. Hay M et al. A novel Angiotensin-(1-7) Glycosylated Mas Receptor Agonist for Treating Vascular Cognitive Impairment and Inflammation-Related Memory Dysfunction. J Pharmacol Exp Ther. 369(1):9-25 (2019).
5. Zhong J C et al. Targeting the apelin pathway as a novel therapeutic approach for cardiovascular diseases. Biochim Biophys Acta Mol Basis Dis. 1863(8):1942-1950 (2017).
6. Zoufaly A et al. Human recombinant soluble ACE2 in severe COVID-19. Lancet Respir Med. 8(11):1154-1158 (2020).
7. Khan A et al. A pilot clinical trial of recombinant human angiotensin-converting enzyme 2 in acute respiratory distress syndrome. Crit Care. 21(1):234 (2017).
8. Batlle D, Wysocki J, Soler M J, Ranganath K, Angiotensin-converting enzyme 2: enhancing the degradation of angiotensin II as a potential therapy for diabetic nephropathy. Kidney Int. 81(6):520-8 (2012).
9. Erster O et al. Site-specific targeting of antibody activity in vivo mediated by disease-associated proteases. J Control Release. 161(3):804-12 (2012).
10. Marquez A, Wysocki J, Pandit J, Batlle D, An update on ACE2 amplification and its therapeutic potential. Acta Physiol (Oxf). Jan; 231(1):e13513 (2021).
11. Wysocki J, Schulze A, Batlle D, Novel Variants of Angiotensin Converting Enzyme-2 of Shorter Molecular Size to Target the Kidney Renin Angiotensin System. Biomolecules. 9(12):886 (2019).
12. Yan R, Zhang Y, Li Y, Xia L, Guo Y, Zhou Q, Structural basis for the recognition of SARS-CoV-2 by full-length human ACE2. Science. 367(6485):1444-1448 (2020).
13. Vickers C et al. Hydrolysis of biological peptides by human angiotensin-converting enzyme-related carboxypeptidase. J Biol Chem. 277(17):14838-43 (2002).
14. Santos R A et al. The ACE2/Angiotensin-(1-7)/MAS Axis of the Renin-Angiotensin System: Focus on Angiotensin-(1-7). Physiol Rev. 98(1):505-553 (2018).
15. Royea J, Hamel E, Brain angiotensin II and angiotensin IV receptors as potential Alzheimer's disease therapeutic targets. Geroscience. 42(5):1237-1256 (2020).
16. Biancardi V C, Son S J, Ahmadi S, Filosa J A, Stem J E, Circulating angiotensin II gains access to the hypothalamus and brain stem during hypertension via breakdown of the blood-brain barrier. Hypertension. 63(3):572-9 (2014).
17. Guy J L, Jackson R M, Jensen H A, Hooper N M, Turner A J, Identification of critical active-site residues in angiotensin-converting enzyme-2 (ACE2) by site-directed mutagenesis. FEBS J.; 272(14):3512-20 (2005).
18. Rushworth C A, Guy J L, Turner A J, Residues affecting the chloride regulation and substrate selectivity of the angiotensin-converting enzymes (ACE and ACE2) identified by site-directed mutagenesis. FEBS J. 275(23):6033-42 (2008).
19. Wang W et al. Angiotensin-Converting Enzyme 2 Metabolizes and Partially Inactivates Pyr-Apelin-13 and Apelin-17: Physiological Effects in the Cardiovascular System. Hypertension. 68(2):365-77 (2016).
20. Masoud A G et al. Apelin directs endothelial cell differentiation and vascular repair following immune-mediated injury. J Clin Invest. 130(1):94-107 (2020).
21. Yavuz Y C et al. Role of Circulating Serum Apelin-13 Levels in Glomerulonephritis: A Pilot Study. J Clin Exp Nephrol 1:2. DOI: 10.21767/2472-5056.100002 (2015).
22. Navar L G, Mitchell K D, Harrison-Bernard L M, Kobori H, Nishiyama A, Review: Intrarenal angiotensin II levels in normal and hypertensive states. J Renin Angiotensin Aldosterone Syst. 2(1sup): S176-S184 (2001).
23. van Kats J P, Schalekamp M A, Verdouw P D, Duncker D J, Danser A H, Intrarenal angiotensin II: interstitial and cellular levels and site of production. Kidney Int. 60(6):2311-7 (2001).
24. Uhal B D, Dang M T, Li X, Abdul-Hafez A, Angiotensinogen gene transcription in pulmonary fibrosis. Int J Pept. 2012; 2012:875910 (2012).
25. Wysocki J et al. A Novel Soluble ACE2 Variant with Prolonged Duration of Action Neutralizes SARS-CoV-2 Infection in Human Kidney Organoids. J Am Soc Nephrol. 32(4):795-803 (2021).
26. Olsen M J et al. Function-based isolation of novel enzymes from a large library. Nat Biotechnol. 18(10):1071-4 (2000).
27. Holstein J M, Gylstorff C, Hollfelder F, Cell-free Directed Evolution of a Protease in Microdroplets at Ultrahigh Throughput. ACS Synth Biol. 10(2):252-257 (2021).
28. Towler P et al. ACE2 X-ray structures reveal a large hinge-bending motion important for inhibitor binding and catalysis. J Biol Chem. 279(17):17996-8007 (2004).
29. Nov Y, When second best is good enough: another probabilistic look at saturation mutagenesis. Appl Environ Microbiol. 78(1):258-62 (2012).
30. Chan K K et al. Engineering human ACE2 to optimize binding to the spike protein of SARS coronavirus 2. Science. 69(6508):1261-1265 (2020).
31. Flores-Muńoz M, Smith N J, Haggerty C, Milligan G, Nicklin S A, Angiotensin-1-9 antagonises pro-hypertrophic signalling in cardiomyocytes via the angiotensin type 2 receptor. J Physiol. 589(Pt 4):939-51 (2011).
32. Jiang F et al. Angiotensin-converting enzyme 2 and angiotensin 1-7: novel therapeutic targets. Nat Rev Cardiol. 11(7):413-26 (2014).
33. Sodhi C P et al. Attenuation of pulmonary ACE2 activity impairs inactivation of des-Arg9 bradykinin/BKB1R axis and facilitates LPS-induced neutrophil infiltration. Am J Physiol Lung Cell Mol Physiol. 314(1): L17-L31 (2018).
34. Fernandes F B et al. Association of Ang-(1-7) and des-Arg9B K as new biomarkers of obesity and cardiometabolic risk factors in adolescents. Hypertens Res. 44(8):969-977 (2021).
35. Gangnus T, Burckhardt B B, Targeted L C-M S/M S platform for the comprehensive determination of peptides in the kallikrein-kinin system. Anal Bioanal Chem. 413(11):2971-2984 (2021).
36. Haschke M et al. Pharmacokinetics and pharmacodynamics of recombinant human angiotensin-converting enzyme 2 in healthy human subjects. Clin Pharmacokinet. 52(9):783-92 (2013).
37. Lubbe L, Cozier G E, Oosthuizen D, Acharya K R, Sturrock E D, ACE2 and ACE: structure-based insights into mechanism, regulation and receptor recognition by SARS-CoV. Clin Sci (Lond). 134(21):2851-2871 (2021).
38. de Graaf M et al. A fully human anti-Ep-CAM scFv-beta-glucuronidase fusion protein for selective chemotherapy with a glucuronide prodrug. Br J Cancer. 86(5):811-8 (2002).
39. Fink T et al. Design of fast proteolysis-based signaling and logic circuits in mammalian cells. Nat Chem Biol. 15(2):115-122 (2019).
40. Shusta E V, Kieke M C, Parke E, Kranz D M, Wittrup K D, Yeast polypeptide fusion surface display levels predict thermal stability and soluble secretion efficiency. J Mol Biol. 292(5):949-56 (1999).
41. Burns M L et al. Directed evolution of brain-derived neurotrophic factor for improved folding and expression in Saccharomyces cerevisiae. Appl Environ Microbiol. 80(18):5732-42 (2014).

1. An unnatural, mutant ACE2 protein comprising an amino acid sequence at least 90% identical to positions 18-615 of SEQ ID NO:1, wherein:
the amino acid sequence comprises one or more of:
a residue other than glutamate at a position corresponding to position 145 of SEQ ID NO:1;
a residue other than asparagine at a position corresponding to position 149 of SEQ ID NO:1;
a residue other than arginine at a position corresponding to position 273 of SEQ ID NO:1;
a residue other than threonine at a position corresponding to position 347 of SEQ ID NO:1;
a residue other than methionine at a position corresponding to position 360 of SEQ ID NO:1;
a residue other than lysine at a position corresponding to position 363 of SEQ ID NO:1;
a residue other than threonine at a position corresponding to position 371 of SEQ ID NO:1;
a residue other than phenylalanine at a position corresponding to position 504 of SEQ ID NO:1;
a residue other than tyrosine at a position corresponding to position 510 of SEQ ID NO:1; and
a residue other than arginine at a position corresponding to position 514 of SEQ ID NO:1; and
the protein exhibits activity in hydrolyzing Angiotensin II.

2. (canceled)

3. The protein of claim 1, wherein the amino acid sequence comprises one or more of:
a residue other than methionine at a position corresponding to position 360 of SEQ ID NO:1;
a residue other than threonine at a position corresponding to position 371 of SEQ ID NO:1; and
a residue other than tyrosine at a position corresponding to position 510 of SEQ ID NO: 1.

4. The protein of claim 1, wherein the amino acid sequence comprises one or more of:
leucine, proline, or a conservative variant of leucine or proline at a position corresponding to position 360 of SEQ ID NO:1;
alanine, aspartate, leucine, phenylalanine, serine, or a conservative variant or alanine, aspartate, leucine, phenylalanine, or serine, at a position corresponding to position 371 of SEQ ID NO:1; and
isoleucine, valine, or a conservative variant of isoleucine or valine at a position corresponding to position 510 of SEQ ID NO:1.

5. The protein of claim 1, wherein the amino acid sequence comprises one or more of:
leucine or proline at a position corresponding to position 360 of SEQ ID NO:1;
alanine, aspartate, leucine, phenylalanine, or serine at a position corresponding to position 371 of SEQ ID NO:1; and
isoleucine or valine at a position corresponding to position 510 of SEQ ID NO:1.

6. The protein of claim 1, wherein the amino acid sequence comprises one or more of:
leucine at a position corresponding to position 360 of SEQ ID NO:1;
leucine at a position corresponding to position 371 of SEQ ID NO:1; and
isoleucine at a position corresponding to position 510 of SEQ ID NO:1.

7. The protein of claim 1, wherein the amino acid sequence comprises two or more of:
a residue other than methionine at a position corresponding to position 360 of SEQ ID NO:1;
a residue other than threonine at a position corresponding to position 371 of SEQ ID NO:1; and
a residue other than tyrosine at a position corresponding to position 510 of SEQ ID NO: 1.

8. The protein of claim 1, wherein the amino acid sequence comprises two or more of:
leucine, proline, or a conservative variant of leucine or proline at a position corresponding to position 360 of SEQ ID NO:1;
alanine, aspartate, leucine, phenylalanine, serine, or a conservative variant or alanine, aspartate, leucine, phenylalanine, or serine, at a position corresponding to position 371 of SEQ ID NO:1; and
isoleucine, valine, or a conservative variant of isoleucine or valine at a position corresponding to position 510 of SEQ ID NO:1.

9. The protein of claim 1, wherein the amino acid sequence comprises two or more of:
leucine or proline at a position corresponding to position 360 of SEQ ID NO:1;
alanine, aspartate, leucine, phenylalanine, or serine at a position corresponding to position 371 of SEQ ID NO:1; and
isoleucine or valine at a position corresponding to position 510 of SEQ ID NO:1.

10. The protein of claim 1, wherein the amino acid sequence comprises two or more of:
leucine at a position corresponding to position 360 of SEQ ID NO:1;
leucine at a position corresponding to position 371 of SEQ ID NO:1; and
isoleucine at a position corresponding to position 510 of SEQ ID NO:1.

11. The protein of claim 1, wherein the amino acid sequence comprises a residue other than tyrosine at a position corresponding to position 510 of SEQ ID NO:1.

12. The protein of claim 11, wherein the amino acid sequence comprises one or more of:
a residue other than methionine at a position corresponding to position 360 of SEQ ID NO:1; and
a residue other than threonine at a position corresponding to position 371 of SEQ ID NO:1.

13-22. (canceled)

23. The protein of claim 1, wherein the amino acid sequence comprises isoleucine at a position corresponding to position 510 of SEQ ID NO:1.

24. The protein of claim 23, wherein the amino acid sequence comprises one or more of:
leucine at a position corresponding to position 360 of SEQ ID NO:1; and
leucine at a position corresponding to position 371 of SEQ ID NO:1.

25-26. (canceled)

27. The protein of claim 1, wherein the amino acid sequence is at least 95% identical to positions 18-615 of SEQ ID NO:1.

28. The protein of claim 1, wherein the protein exhibits at least one of:
an increase in k_{cat} in hydrolyzing Angiotensin II;
a decrease in Kₘ in hydrolyzing Angiotensin II;
an increase in k_{cat}/Kₘ in hydrolyzing Angiotensin II;
an increase in Angiotensin II:Apelin-13 hydrolysis ratio;
a decrease in k_{cat}/Kₘ in hydrolyzing Apelin-13; and
a decrease in k_{cat}/Kₘ in hydrolyzing Des-Arg9-bradykinin,
with respect to a protein comprising an amino acid sequence 100% identical to positions 18-615 of SEQ ID NO:1.

29. A recombinant polynucleotide encoding the protein of claim 1.

30. A vector comprising the recombinant polynucleotide of claim 29.

31. A recombinant cell comprising the recombinant polynucleotide of claim 29.

32. A method of treating an ACE2-sensitive condition, the method comprising administering to a subject in need thereof the protein of claim 1 in an amount effective to treat the ACE2-sensitive condition.

33. The protein of claim 1, wherein the amino acid sequence is at least 95% identical to positions 18-615 of SEQ ID NO:1 and comprises:
isoleucine at a position corresponding to position 510 of SEQ ID NO:1;
leucine at a position corresponding to position 360 of SEQ ID NO:1 and isoleucine at a position corresponding to position 510 of SEQ ID NO:1; or
leucine at a position corresponding to position 371 of SEQ ID NO:1 and isoleucine at a position corresponding to position 510 of SEQ ID NO:1.


SEQUENCE LISTING (18 sequences)

SEQ ID NO: 1 - protein, 805 aa, Homo sapiens [sequence omitted]

SEQ ID NO: 2 - DNA, 2418 bp, Homo sapiens
  source 1..2418: mol_type = other DNA
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 3 - protein, 10 aa, Homo sapiens
DRVYIHPFHL

SEQ ID NO: 4 - protein, 13 aa, Homo sapiens
QRPRLSHKGPMPF

SEQ ID NO: 5 - protein, 9 aa, Homo sapiens
RPPGFSPFR

SEQ ID NO: 6 - protein, 9 aa, Homo sapiens
KRPPGFSPF

SEQ ID NO: 7 - protein, 7 aa, Homo sapiens
YPFVEPI

SEQ ID NO: 8 - protein, 6 aa, Homo sapiens
YPVEPI

SEQ ID NO: 9 - protein, 13 aa, Homo sapiens
YGGFLRRIRPKLK

SEQ ID NO: 10 - protein, 12 aa, Homo sapiens
ESKKPPAKLQPR

SEQ ID NO: 11 - protein, 13 aa, Homo sapiens
  MOD_RES 1: note = pyroglyutamic acid
XLYENKPRRPYIL

SEQ ID NO: 12 - DNA, 1794 bp, synthetic construct
  source 1..1794: mol_type = other DNA
caatctaccatcgaggagcaagctaagactttcctagataagttcaaccatgaggccgaagacttattttaccagagctcactagcaagctggaactataacactaacataacagaggagaatgtgcaaaacatgaataatgctggcgataagtggagtgccttcttgaaggagcaaagtacccttgcccagatgtatcctctacaggaaatacaaaacctgacggttaagctacaattacaagctctgcaacaaaatgggtcaagtgttttgtccgaagacaagagtaaaaggcttaatacgatcctaaacacgatgagcaccatctactctacggggaaggtctgtaaccccgacaaccctcaagagtgtctgctgctggaaccaggtttgaacgagatcatggccaactcattggattataatgagcgtctgtgggcctgggaatcctggaggtccgaggtggggaaacaacttagacccctttacgaggagtacgttgttctaaagaacgaaatggcaagggctaatcactacgaggattacggcgactactggagaggggactatgaagtaaacggtgtagacggctacgattatagcaggggccagctgatcgaagatgtagagcatactttcgaggagattaagccactgtatgagcatttgcatgcatatgtaagggccaagttaatgaacgcttacccctcttatatatctcccatcggatgtctgcctgctcacttacttggggacatgtgggggcgtttctggactaacttatactccctgacagtaccctttggtcaaaagccgaatatagacgtaacggacgctatggtggaccaagcgtgggacgcgcagagaattttcaaggaggcggagaagttttttgtatcagtgggccttccgaacatgactcaaggattttgggagaattctatgctaaccgacccaggcaacgtgcaaaaagcagtctgccaccccaccgcatgggatctgggaaaaggggattttcgtatactaatgtgtacaaaagtcacgatggatgatttcttaacggcacaccatgagatgggccatatacagtacgacatggcatatgcggcccagccttttctattgaggaacggggccaacgagggtttccatgaagcggttggagagataatgtcactaagtgcagcgacgcccaagcatctgaaaagcattgggctgttaagcccggactttcaggaagacaacgaaacggaaattaactttttgctaaaacaggcactgacgatcgttggcactctaccctttacgtatatgttagaaaagtggaggtggatggtttttaagggtgaaattcccaaagaccaatggatgaagaaatggtgggaaatgaagagggaaattgtcggcgtggttgagccggtgcctcacgacgagacttactgtgacccggcctctttgttccacgtatccaatgattacagctttattaggtactacactcgtacgttatatcaattccagtttcaagaagcgctgtgccaggcagctaagcatgaaggtcctttacacaaatgcgacatatctaactcaactgaggcgggtcagaaacttttcaatatgctaaggctaggtaaatcagagccatggactctagctttagagaacgtagtgggtgccaagaacatgaacgtgagaccactgctaaactattttgaaccactgttcacttggcttaaggatcagaacaagaatagttttgtcggatggtctacagactggtccccctatgcagac

SEQ ID NO: 13 - DNA, 22 bp, synthetic construct
  source 1..22: mol_type = other DNA
gtcttgttggctatcttcgctg

SEQ ID NO: 14 - DNA, 19 bp, synthetic construct
  source 1..19: mol_type = other DNA
gtcgttgacaaagagtacg

SEQ ID NO: 15 - DNA, 20 bp, synthetic construct
  source 1..20: mol_type = other DNA
ggactttcaggaagacaacg

SEQ ID NO: 16 - DNA, 22 bp, synthetic construct
  source 1..22: mol_type = other DNA
cgtggatagcggtttgactcac

SEQ ID NO: 17 - DNA, 21 bp, synthetic construct
  source 1..21: mol_type = other DNA
cctactcagacaatgcgatgc

SEQ ID NO: 18 - DNA, 22 bp, synthetic construct
  source 1..22: mol_type = other DNA
gttactgatgcaatggtggacc